(19) United States
(12) Patent Application Publication
(10) Pub. No.: US 2022/0388978 A1
(43) Pub. Date: Dec. 8, 2022

(54) CEREBLON E3 LIGASE INHIBITORS

(71) Applicant: REGENTS OF THE UNIVERSITY OF MICHIGAN, Ann Arbor, MI (US)

(72) Inventors: Shaomeng Wang, Superior Township, MI (US); Tianfeng Xu, Ypsilanti, MI (US); Mingliang Wang, Ann Arbor, MI (US); Jiantao Hu, Ann Arbor, MI (US); Xi Han, Ann Arbor, MI (US); Weiguo Xiang, Ypsilanti, MI (US); Rohan Rej, Ann Arbor, MI (US)

(21) Appl. No.: 17/633,977

(22) PCT Filed: Aug. 27, 2020
(86) PCT No.: PCT/US2020/048186

§ 371 Date: Feb. 9, 2022

Related U.S. Application Data
(60) Provisional application No. 62/892,144, filed on Aug. 27, 2019.
(60) Provisional application No. 63/024,719, filed on May 14, 2020.

(51) Int. Cl. C07D 401/04; C07D 471/10; C07D 487/04; C07D 487/10

(52) U.S. Cl. CPC C07D 401/04; C07D 471/10; C07D 487/04; C07D 487/10

(57) ABSTRACT

The present disclosure provides compounds represented by Formula I: wherein R2a, R2b, R2c, R2d, R3, R13, and Z are as defined in the specification, and the salts and solvates thereof. Compounds of Formula I are cereblon (CRBN) ubiquitination inhibitors or monofunctional synthetic intermediates that can be used to prepare PROTAC molecules. CRBN ubiquitination inhibitors and PROTAC molecules are useful for the treatment of cancer and other diseases.

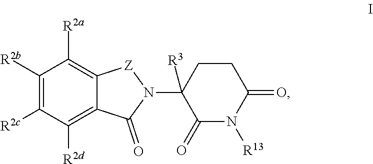

### BACKGROUND OF THE INVENTION

### Field of the Invention

The present disclosure provides cereblon (CRBN) ubiquitination inhibitors and therapeutic methods of treating conditions and diseases, e.g., cancer, wherein the inhibition of CRBN ubiquitination provides a benefit.

### Background

Cereblon (CRBN), a component of the DDB1-CUL4a-Rocl ubiquitin ligase complex, is a molecular target of immunomodulatory agents such as thalidomide, lenalidomide, and pomalidomide. Lopez-Girona et al., Leukemia 26:2326-2335 (2012). Inhibition of CRBN ubiquitination by these agents may allow CRBN to accumulate, leading to the increased cullin-4 RING E3 ligase-mediated degradation of target proteins. Liu et al., FASEB J 12:4829-4839 (2015). There exists a need for new immunomodulatory agents for the treatment of cancer and other diseases.

### BRIEF SUMMARY OF THE INVENTION

In one aspect, the present disclosure provides compounds represented by any one of Formulae I-IV, IX-XVI, or XVIII-XXII, below, and the pharmaceutically acceptable salts and solvates, e.g., hydrates, thereof, collectively referred to as “Compounds of the Disclosure.” Compounds of the Disclosure inhibit CRBN ubiquitination and are thus useful in treating or preventing diseases or conditions such as cancer wherein the inhibition of CRBN ubiquitination provides a benefit. Compounds of the Disclosure may also be synthetic intermediates that are used to prepare CRBN ubiquitination inhibitors. Compounds of the Disclosure may also be synthetic intermediates that are used to prepare targeted-protein degraders.

In another aspect, the present disclosure provides methods of treating or preventing a condition or disease by administering a therapeutically effective amount of a Compound of the Disclosure to a subject, e.g., a human patient, in need thereof. The disease or condition of interest that is treatable or preventable by inhibition CRBN ubiquitination is, for example, cancer or other proliferative disorder, or an inflammatory disease. Also provided are methods of preventing the proliferation of unwanted proliferating cells, such as in cancer, in a subject comprising administering a therapeutically effective amount of a Compound of the Disclosure to a subject at risk of developing a condition characterized by unwanted proliferating cells. In some embodiments, Compounds of the Disclosure may reduce the proliferation of unwanted cells by modulating the function of CRBN in those cells. In some embodiments, Compounds of the Disclosure are administered in combination with an optional therapeutic agent.

In another aspect, the present disclosure provides a method of inhibiting CRBN ubiquitination in a subject, comprising administering to the subject a therapeutically effective amount of a Compound of the Disclosure.

In another aspect, the present disclosure provides a pharmaceutical composition comprising a Compound of the Disclosure and an excipient and/or pharmaceutically acceptable carrier.

In another aspect, the present disclosure provides a composition comprising a Compound of the Disclosure and an excipient and/or pharmaceutically acceptable carrier for use treating or preventing diseases or conditions wherein the inhibition of CRBN ubiquitination provides a benefit, e.g., cancer.

In another aspect, the present disclosure provides a composition comprising: (a) a Compound of the Disclosure; (b) a second therapeutically active agent; and (c) optionally an excipient and/or pharmaceutically acceptable carrier.

In another aspect, the present disclosure provides a Compound of the Disclosure for use in the treatment or prevention of a disease or condition of interest, e.g., cancer.

In another aspect, the present disclosure provides a use of a Compound of the Disclosure for the manufacture of a medicament for treating a disease or condition of interest, e.g., cancer.

In another aspect, the present disclosure provides a kit comprising a Compound of the Disclosure, and, optionally, a packaged composition comprising an optional therapeutic agent useful in the treatment of a disease or condition of interest, and a package insert containing directions for use in the treatment of a disease or condition, e.g., cancer.

In one aspect, the present disclosure provides compounds represented by any one of Formulae VI-VIII or XVII, below, and the salts and solvates, e.g., hydrates, thereof, collectively referred to as “Intermediates of the Disclosure.” Intermediates of the Disclosure can be used to prepare Compounds of the Disclosure.

In another aspect, the present disclosure provides methods of preparing Compounds of the Disclosure.

In another embodiment, the disclosure provides compounds represented by any one of Formulae XXIII-XXXIV, below, and the pharmaceutically acceptable salts and solvates, e.g., hydrates, thereof, collectively referred to as “PROTAC Molecules.” A PROTAC molecule is a heterobifunctional small molecule containing a ligand that binds to a target protein of interest and a second ligand for an E3 ligase covalently tethered to one another by a chemical linker.

In another aspect, the present disclosure provides methods of preparing PROTAC molecules comprising Compounds of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF THE INVENTION

### I. Compounds of the Disclosure

Compounds of the Disclosure inhibit the ubiquitination of CRBN. Without wishing to be bound by any particular theory, inhibition of CRBN ubiquitination may allow CRBN to accumulate, leading to the increased cullin-4 RING E3 ligase-mediated degradation of target proteins. See Liu et al., FASEB J 29:4829-4839 (2015).

Compounds of the Disclosure may also be used as monofunctional synthetic intermediates to prepare PROTAC Molecules.

In one embodiment, Compounds of the Disclosure are compounds of Formula I:

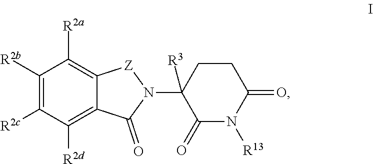

wherein:

R^{2b} and R^{2c} are taken together to form a —(CH₂)ₘ—N(R¹)—(CH₂)ₙ— radical, a —(CH₂)ₘ—C(R^{1a})(R^{1b})—(CH₂)ₙ— radical, or a

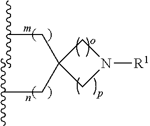

radical; and R^{2a} and R^{2a} and R^{2d} are independently selected from the group consisting of hydrogen, halo, C₁-C₃ alkyl, and C₁-C₃ alkoxy; or

R^{2a} and R^{2b} are taken together to form a —(CH₂)ₘ—N(R¹)—(CH₂)ₙ— radical, a —(CH₂)ₘ—C(R^{1a})(R^{1b})—(CH₂)ₙ— radical, or a

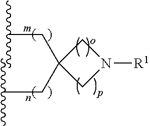

radical; and R^{2c} and R^{2d} are independently selected from the group consisting of hydrogen, halo, C₁-C₃ alkyl, and C₁-C₃ alkoxy; or

R^{2c} and R^{2d} are taken together to form a —(CH₂)ₘ—N(R¹)—(CH₂)ₙ— radical, a —(CH₂)ₘ—C(R^{1a})(R^{1b})—(CH₂)ₙ— radical, or a

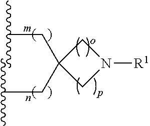

radical; and R^{2a} and R^{2b} are independently selected from the group consisting of hydrogen, halo, C₁-C₃ alkyl, and C₁-C₃ alkoxy;

R³ is selected from the group consisting of hydrogen, deuterium, fluoro, and C₁-C₃ alkyl;

m is 1, 2, or 3;

n is 1, 2, or 3;

o is 1, 2, or 3;

p is 1, 2, or 3;

Z is selected from the group consisting of —CR^{8a}R^{8b}— and —C(═O)—;

R¹ is selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷;

R^{1a} is selected from the group consisting of hydrogen, —OH, —CHO, —C(═O)OH, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷;

R^{1b} is selected from the group consisting of hydrogen and C₁-C₃ alkyl; or

R^{1a} and R^{1b} taken together with the carbon atom to which they are attached form a —C(═O)—;

R⁴ is selected from the group consisting of —R^{4a}, —OR^{4b}, and —NR^{4c}R^{4d.};

R⁵ is selected from the group consisting of —R^{5a} and —NR^{5a}R^{5b};

R⁶ is selected from the group consisting of hydrogen, C₁-C₆ alkyl, and cyano;

R⁷ is selected from the group consisting of hydrogen, C₁-C₆ alkyl, and —NR^{7a}R^{7b};

R^{4a} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;

R^{4b} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;

R^{4c} and R^{4d} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl; or

R^{4c} and R^{4d} taken together with the nitrogen atom to which they are attached form a 4- to 8-membered optionally substituted heterocyclo;

R^{5a} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;

R^{5b} and R^{5c} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;

R^{7a} and R^{7b} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl; or

R^{7a} and R^{7b} taken together with the nitrogen atom to which they are attached form a 4- to 8-membered optionally substituted heterocyclo;

R^{8a} and R^{8b} are independently selected from the group consisting of hydrogen and C₁-C₃ alkyl; or

R^{8a} and R^{8b} taken together with the carbon atom to which they are attached from a C₃-C₆ cycloalkyl; and

R¹³ is selected from the group consisting of hydrogen and C₁-C₃ alkyl, or a pharmaceutically acceptable salt or solvate thereof.

In another embodiment, Compounds of the Disclosure are compounds of Formula I, wherein Z is selected from the group consisting of —CH₂— and —C(═O)—, or a pharmaceutically acceptable salt or solvate thereof.

In another embodiment, Compounds of the Disclosure are compounds of Formula I, wherein R¹³ is hydrogen, or a pharmaceutically acceptable salt or solvate thereof.

In another embodiment, Compounds of the Disclosure are compounds of Formula I, wherein R¹³ is methyl, or a pharmaceutically acceptable salt or solvate thereof.

In another embodiment, Compounds of the Disclosure are compounds of Formula II:

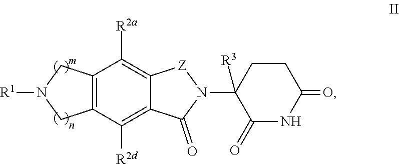

wherein R¹, R^{2a}, R^{2d}, R³, m n, and Z are as defined in connection with Formula I, or a pharmaceutically acceptable salt or solvate thereof.

In another embodiment, Compounds of the Disclosure are compounds of Formula II, wherein Z is —CH₂—, or a pharmaceutically acceptable salt or solvate thereof.

In another embodiment, Compounds of the Disclosure are compounds of Formula II, wherein Z is —C(═O)—, or a pharmaceutically acceptable salt or solvate thereof.

In another embodiment, Compounds of the Disclosure are compounds of Formula II, wherein R^{2a} and R^{2d} are independently selected from the group consisting of hydrogen, fluoro, and chloro, or a pharmaceutically acceptable salt or solvate thereof. In another embodiment, R^{2a} and R^{2d} are hydrogen.

In another embodiment, Compounds of the Disclosure are compounds of Formula III:

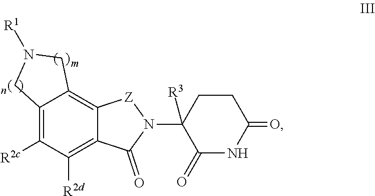

wherein R¹, R^{2c},, R^{2d}, R³, m n, and Z are as defined in connection with Formula I, or a pharmaceutically acceptable salt or solvate thereof.

In another embodiment, Compounds of the Disclosure are compounds of Formula III, wherein Z is —CH₂—, or a pharmaceutically acceptable salt or solvate thereof.

In another embodiment, Compounds of the Disclosure are compounds of Formula III, wherein Z is —C(═O)—, or a pharmaceutically acceptable salt or solvate thereof.

In another embodiment, Compounds of the Disclosure are compounds of Formula III, wherein R^{2c} and R^{2d} are independently selected from the group consisting of hydrogen, fluoro, and chloro, or a pharmaceutically acceptable salt or solvate thereof. In another embodiment, R^{2c} and R^{2d} are hydrogen.

In another embodiment, Compounds of the Disclosure are compounds of Formula IV:

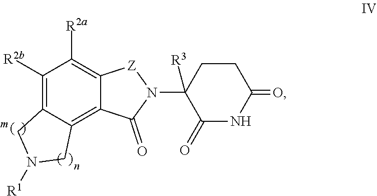

wherein R¹, R^{2a}, R^{2b}, R³, m n, and Z are as defined in connection with Formula I; and Z is —CR^{8a}R^{8b}—, or a pharmaceutically acceptable salt or solvate thereof.

In another embodiment, Compounds of the Disclosure are compounds of Formula IV, wherein Z is —CH₂—, or a pharmaceutically acceptable salt or solvate thereof.

In another embodiment, Compounds of the Disclosure are compounds of Formula IV, wherein R^{2a} and R^{2b} are independently selected from the group consisting of hydrogen, fluoro, and chloro, or a pharmaceutically acceptable salt or solvate thereof. In another embodiment, R^{2a} and R^{2b} are hydrogen.

In another embodiment, Compounds of the Disclosure are compounds of Formula IX:

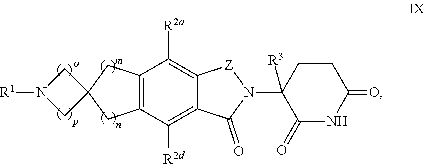

wherein R¹, R^{2a}, R^{2d}, R³, m n, o, p, and Z are as defined in connection with Formula I, or a pharmaceutically acceptable salt or solvate thereof.

In another embodiment, Compounds of the Disclosure are compounds of Formula IX, wherein Z is —CH₂—, or a pharmaceutically acceptable salt or solvate thereof.

In another embodiment, Compounds of the Disclosure are compounds of Formula IX, wherein Z is —C(═O)—, or a pharmaceutically acceptable salt or solvate thereof.

In another embodiment, Compounds of the Disclosure are compounds of Formula IX, wherein R^{2a} and R^{2d} are independently selected from the group consisting of hydrogen, fluoro, and chloro, or a pharmaceutically acceptable salt or solvate thereof. In another embodiment, R^{2a} and R^{2d} are hydrogen.

In another embodiment, Compounds of the Disclosure are compounds of Formula X:

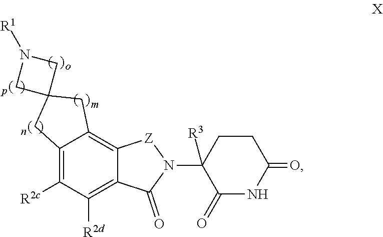

wherein R¹, R^{2c},, R^{2d}, R³, m n, o, p, and Z are as defined in connection with Formula I, or a pharmaceutically acceptable salt or solvate thereof.

In another embodiment, Compounds of the Disclosure are compounds of Formula X, wherein Z is —CH₂—, or a pharmaceutically acceptable salt or solvate thereof.

In another embodiment, Compounds of the Disclosure are compounds of Formula X, wherein Z is —C(═O)—, or a pharmaceutically acceptable salt or solvate thereof.

In another embodiment, Compounds of the Disclosure are compounds of

Formula X, wherein R^{2c} and R^{2d} are independently selected from the group consisting of hydrogen, fluoro, and chloro, or a pharmaceutically acceptable salt or solvate thereof. In another embodiment, R^{2c} and R^{2d} are hydrogen.

In another embodiment, Compounds of the Disclosure are compounds of Formula XI:

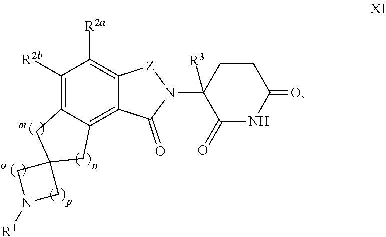

wherein R¹, R^{2a}, R^{2b}, R³, m n, o, and p are as defined in connection with Formula I; and Z is —CR^{8a}R^{8b}—, or a pharmaceutically acceptable salt or solvate thereof.

In another embodiment, Compounds of the Disclosure are compounds of Formula XI, wherein Z is —CH₂—, or a pharmaceutically acceptable salt or solvate thereof.

In another embodiment, Compounds of the Disclosure are compounds of Formula XI, wherein R^{2a} and R^{2b} are independently selected from the group consisting of hydrogen, fluoro, and chloro, or a pharmaceutically acceptable salt or solvate thereof. In another embodiment, R^{2a} and R^{2b} are hydrogen.

In another embodiment, Compounds of the Disclosure are compounds of Formula XII:

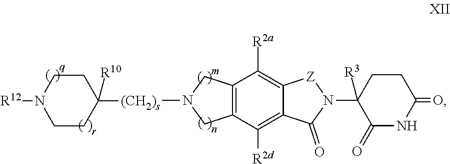

wherein:

q and r are independently 0, 1, or 2;

s is 0 or 1;

R¹⁰ is selected from the group consisting of hydrogen, halo, C₁-C₃ alkyl, and C₁-C₃ alkoxy;

R¹² is selected from the group consisting of hydrogen, optionally substituted heterocyclo, and optionally substituted phenyl; and

R^{2a}, R^{2d}, R³, m, n, and Z are as defined in connection with Formula I, or a pharmaceutically acceptable salt or solvate thereof.

In another embodiment, Compounds of the Disclosure are compounds of Formula XIII:

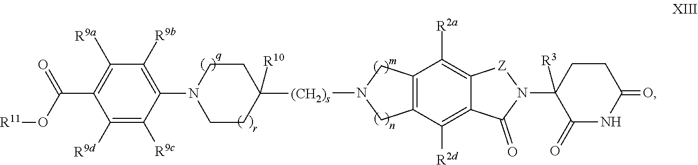

wherein:

q and r are independently 0, 1, or 2;

s is 0 or 1;

R^{9a} , R^{9b}, R^{9c},and R^{9d} are independently selected from the group consisting of hydrogen, halo, C₁-C₃ alkyl, C₁-C₃ haloalkyl, and C₁-C₃ alkoxy;

R¹⁰ is selected from the group consisting of hydrogen, halo, C₁-C₃ alkyl, and C₁-C₃ alkoxy;

R¹¹ is selected from the group consisting of hydrogen and C₁-C₆ alkyl; and

R^{2a}, R^{2d}, R³, m, n, and Z are as defined in connection with Formula I, or a pharmaceutically acceptable salt or solvate thereof.

In another embodiment, Compounds of the Disclosure are compounds of Formula XIV:

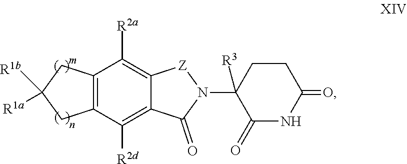

wherein R^{1a}, R^{1b}, R^{2a}, R^{2d}, R³, m, n, and Z are as defined in connection with Formula I, or a pharmaceutically acceptable salt or solvate thereof.

In another embodiment, Compounds of the Disclosure are compounds of Formula XIV, wherein Z is —CH₂—, or a pharmaceutically acceptable salt or solvate thereof.

In another embodiment, Compounds of the Disclosure are compounds of Formula XIV, wherein Z is —C(═O)—, or a pharmaceutically acceptable salt or solvate thereof.

In another embodiment, Compounds of the Disclosure are compounds of Formula XIV, wherein R^{2a} and R^{2d} are independently selected from the group consisting of hydrogen, fluoro, and chloro, or a pharmaceutically acceptable salt or solvate thereof. In another embodiment, R^{2a} and R^{2d} are hydrogen.

In another embodiment, Compounds of the Disclosure are compounds of Formula XV:

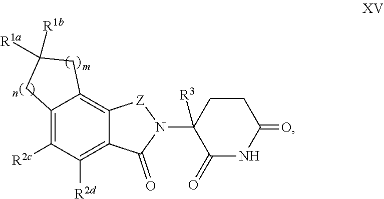

wherein R^{1a}, R^{1b}, R^{2c}, R^{2d}, R³, m, n, and Z are as defined in connection with Formula I, or a pharmaceutically acceptable salt or solvate thereof.

In another embodiment, Compounds of the Disclosure are compounds of Formula XV, wherein Z is —CH₂—, or a pharmaceutically acceptable salt or solvate thereof.

In another embodiment, Compounds of the Disclosure are compounds of Formula XV, wherein Z is —C(═O)—, or a pharmaceutically acceptable salt or solvate thereof.

In another embodiment, Compounds of the Disclosure are compounds of Formula XV, wherein R^{2c} and R^{2d} are independently selected from the group consisting of hydrogen, fluoro, and chloro, or a pharmaceutically acceptable salt or solvate thereof. In another embodiment, R^{2c} and R^{2d} are hydrogen.

In another embodiment, Compounds of the Disclosure are compounds of Formula XVI:

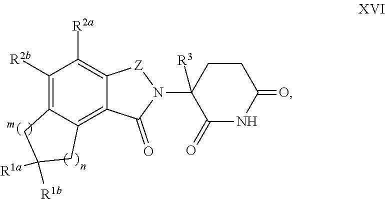

wherein R^{1a}, R^{1b}, R^{2a}, R^{2b}, R³, m, and n are as defined in connection with Formula I; and Z is —CR^{8a}R^{8b}—, or a pharmaceutically acceptable salt or solvate thereof.

In another embodiment, Compounds of the Disclosure are compounds of Formula XVI, wherein Z is —CH₂—, or a pharmaceutically acceptable salt or solvate thereof.

In another embodiment, Compounds of the Disclosure are compounds of Formula XVI, wherein R^{2a} and R^{2b} are independently selected from the group consisting of hydrogen, fluoro, and chloro, or a pharmaceutically acceptable salt or solvate thereof. In another embodiment, R^{2a} and R^{2b} are hydrogen.

In another embodiment, Compounds of the Disclosure are compounds of Formula XVIII:

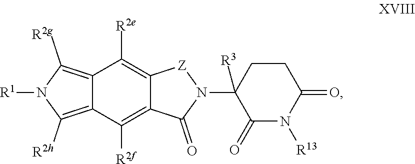

wherein:

R^{2e}, R^{2f}, R^{2g}, and R^{2h} are independently selected from the group consisting of hydrogen, halo, C₁-C₃ alkyl, and C₁-C₃ alkoxy; and

R¹, R³, R¹³, and Z as defined in connection with Formula I, or a pharmaceutically acceptable salt or solvate thereof.

In another embodiment, Compounds of the Disclosure are compounds of Formula XVIII, wherein Z is —CH₂—, or a pharmaceutically acceptable salt or solvate thereof.

In another embodiment, Compounds of the Disclosure are compounds of Formula XVIII, wherein Z is —C(═O)—, or a pharmaceutically acceptable salt or solvate thereof.

In another embodiment, Compounds of the Disclosure are compounds of Formula XVIII, wherein R^{2e} and R^{2f} are independently selected from the group consisting of hydrogen and halo, or a pharmaceutically acceptable salt or solvate thereof. In another embodiment, R^{2e} and R^{2f} are hydrogen.

In another embodiment, Compounds of the Disclosure are compounds of Formula XVIII, wherein R^{2g} and R^{2h} are independently selected from the group consisting of hydrogen, halo, and C₁-C₃ alkyl, or a pharmaceutically acceptable salt or solvate thereof. In another embodiment, R^{2g} and R^{2h} are hydrogen.

In another embodiment, Compounds of the Disclosure are compounds of Formula XIX:

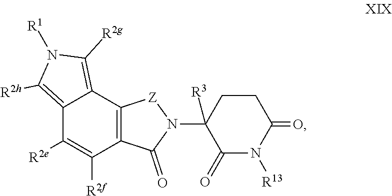

wherein:

R^{2e}, R^{2f}, R^{2g}, and R^{2h} are independently selected from the group consisting of hydrogen, halo, C₁-C₃ alkyl, and C₁-C₃ alkoxy; and

R¹, R³, R¹³, and Z as defined in connection with Formula I, or a pharmaceutically acceptable salt or solvate thereof.

In another embodiment, Compounds of the Disclosure are compounds of Formula XIX, wherein Z is —CH₂—, or a pharmaceutically acceptable salt or solvate thereof.

In another embodiment, Compounds of the Disclosure are compounds of Formula XIX, wherein Z is —C(═O)—, or a pharmaceutically acceptable salt or solvate thereof.

In another embodiment, Compounds of the Disclosure are compounds of Formula XIX, wherein R^{2e} and R^{2f} are independently selected from the group consisting of hydrogen and halo, or a pharmaceutically acceptable salt or solvate thereof. In another embodiment, R^{2e} and R^{2f} are hydrogen.

In another embodiment, Compounds of the Disclosure are compounds of Formula XIX, wherein R^{2g} and R^{2h} are independently selected from the group consisting of hydrogen, halo, and C₁-C₃ alkyl, or a pharmaceutically acceptable salt or solvate thereof. In another embodiment, R^{2g} and R^{2h} are hydrogen

In another embodiment, Compounds of the Disclosure are compounds of Formula XX:

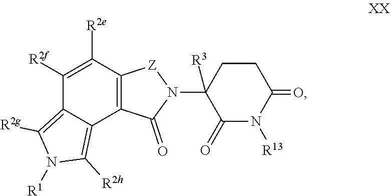

wherein:

R^{2e}, R^{2f}, R^{2g}, and R^{2h} are independently selected from the group consisting of hydrogen, halo, C₁-C₃ alkyl, and C₁-C₃ alkoxy;

Z is —CR^{8a}R^{8b}—; and

R¹, R³, and R¹³ as defined in connection with Formula I, or a pharmaceutically acceptable salt or solvate thereof.

In another embodiment, Compounds of the Disclosure are compounds of Formula XX, wherein Z is —CH₂—, or a pharmaceutically acceptable salt or solvate thereof.

In another embodiment, Compounds of the Disclosure are compounds of Formula XX, wherein R^{2e} and R^{2f} are independently selected from the group consisting of hydrogen and halo, or a pharmaceutically acceptable salt or solvate thereof. In another embodiment, R^{2e} and R^{2f} are hydrogen.

In another embodiment, Compounds of the Disclosure are compounds of Formula XX, wherein R^{2g} and R^{2h} are independently selected from the group consisting of hydrogen, halo, and C₁-C₃ alkyl, or a pharmaceutically acceptable salt or solvate thereof. In another embodiment, R^{2g} and R^{2h} are hydrogen.

In another embodiment, Compounds of the Disclosure are compounds of Formula XXI:

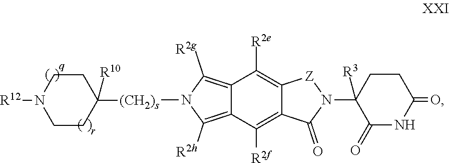

wherein:

R^{2e}, R^{2f}, R^{2g}, and R^{2h} are as defined in connection with Formula XVIII;

q, r, s, R¹⁰, and R¹², are as defined in connection with Formula XII; and

R³ and Z as defined in connection with Formula I, or a pharmaceutically acceptable salt or solvate thereof.

In another embodiment, Compounds of the Disclosure are compounds of Formula XXII:

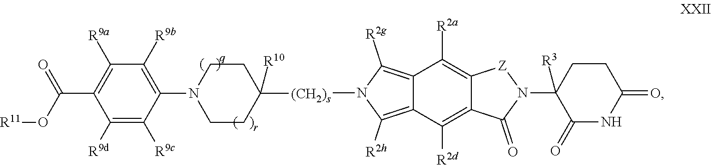

wherein:

R^{2e}, R^{2f}, R^{2g}, and R^{2h} are as defined in connection with Formula XVIII;

q, r, s, R^{9a}, R^{9b}, R^{9c}, R^{9d}, R¹⁰, R¹¹, and R¹² are as defined in connection with Formula XIII; and

R³ and Z as defined in connection with Formula I, or a pharmaceutically acceptable salt or solvate thereof.

In another embodiment, Compounds of the Disclosure are compounds of any one of Formulae I-IV, IX-XVI, or XVIII-XXII, and PROTAC Molecules are compounds of any one of Formula XXIII-XXXIV, see below, wherein R³ is selected from the group consisting of hydrogen, deuterium, fluoro, and methyl, or a pharmaceutically acceptable salt or solvate thereof. In another embodiment, R³ is hydrogen. In another embodiment, R³ is deuterium. In another embodiment, R³ is fluoro. In another embodiment, R³ is methyl.

In another embodiment, Compounds of the Disclosure are compounds of any one of Formulae I-IV or IX-XVI, and PROTAC Molecules are compounds of any one of Formula XXIII-XXXI, wherein m is 1, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, wherein m is 2, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, wherein m is 3, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, wherein n is 1, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, wherein n is 2, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, wherein n is 3, or a pharmaceutically acceptable salt or solvate thereof

In another embodiment, Compounds of the Disclosure are compounds of any one of Formulae I-IV or IX-XVI, and PROTAC Molecules are compounds of any one of Formula XXIII-XXXI, wherein m is 1 and n is 1, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, see below, wherein m is 1 and n is 2, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, wherein m is 2 and n is 1, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, wherein m is 2 and n is 2, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, wherein m is 1 and n is 3, or a pharmaceutically acceptable salt or solvate thereof.

In another embodiment, Compounds of the Disclosure are compounds of any one of Formulae I-IV or IX-XVI, and PROTAC Molecules are compounds of any one of Formula XXIII-XXXI, wherein m is 3 and n is 1, or a pharmaceutically acceptable salt or solvate thereof.

In another embodiment, Compounds of the Disclosure are compounds of any one of Formulae I-IV, IX-XI, or XVIII-XX, wherein R¹ is hydrogen, or a pharmaceutically acceptable salt or solvate thereof.

In another embodiment, Compounds of the Disclosure are compounds of any one of Formulae XIV-XVI, wherein R^{1a} is selected from the group consisting of —OH, —CHO, —CH₂OH, and —C(═O)OH; and R^{1b} is hydrogen, or a pharmaceutically acceptable salt or solvate thereof.

In another embodiment, Compounds of the Disclosure are compounds of any one of Formulae XIV-XVI, wherein R^{1a} and R^{1b} taken together with the carbon atom to which they are attached form a —C(═O)—, or a pharmaceutically acceptable salt or solvate thereof.

In another embodiment, Compounds of the Disclosure are compounds of any one of Formulae IX-XI, and PROTAC Molecules are compounds of any one of Formula XXVI-XXVIII, wherein o is 1, or a pharmaceutically acceptable salt or solvate thereof.

In another embodiment, Compounds of the Disclosure are compounds of any one of Formulae IX-XI, and PROTAC Molecules are compounds of any one of Formula XXVI-XXVIII, wherein o is 2, or a pharmaceutically acceptable salt or solvate thereof.

In another embodiment, Compounds of the Disclosure are compounds of any one of Formulae I IX-XI, and PROTAC Molecules are compounds of any one of Formula XXVI-XXVIII, wherein p is 1, or a pharmaceutically acceptable salt or solvate thereof.

In another embodiment, Compounds of the Disclosure are compounds of any one of Formulae IX-XI, and PROTAC Molecules are compounds of any one of Formula XXVI-XXVIII, wherein p is 2, or a pharmaceutically acceptable salt or solvate thereof.

In another embodiment, Compounds of the Disclosure are compounds of any one of Formulae IX-XI, and PROTAC Molecules are compounds of any one of Formula XXVI-XXVIII, wherein o is 1 and p is 1, or a pharmaceutically acceptable salt or solvate thereof.

In another embodiment, Compounds of the Disclosure are compounds of any one of Formulae IX-XI, and PROTAC Molecules are compounds of any one of Formula XXVI-XXVIII, wherein o is 1 and p is 2, or a pharmaceutically acceptable salt or solvate thereof.

In another embodiment, Compounds of the Disclosure are compounds of any one of Formulae IX-XI, and PROTAC Molecules are compounds of any one of Formula XXVI-XXVIII, wherein o is 2 and p is 1, or a pharmaceutically acceptable salt or solvate thereof.

In another embodiment, Compounds of the Disclosure are compounds of any one of Formulae IX-XI, and PROTAC Molecules are compounds of any one of Formula XXVI-XXVIII, see below, wherein o is 2 and p is 2, or a pharmaceutically acceptable salt or solvate thereof.

In another embodiment, Compounds of the Disclosure are compounds of any one of Formulae I-IV, IX-XI, or XVIII-XX, wherein R¹ is selected from the group consisting of C₁-C₆ alkyl, C₁-C₆ haloalkyl, optionally substituted C₃-C₈ cycloalkyl, or a pharmaceutically acceptable salt or solvate thereof.

In another embodiment, Compounds of the Disclosure are compounds of any one of Formulae I-IV, IX-XI, or XVIII-XX, wherein R¹ is C₁-C₆ alkyl, or a pharmaceutically acceptable salt or solvate thereof.

In another embodiment, Compounds of the Disclosure are compounds of any one of Formulae I-IV, IX-XI, or XVIII-XX, wherein R¹ is optionally substituted C₁-C₆ alkyl, or a pharmaceutically acceptable salt or solvate thereof. In another embodiment, R¹ is carboxyalkyl, e.g., —CH₂C(═O)OH.

In another embodiment, Compounds of the Disclosure are compounds of any one of Formulae I-IV, IX-XI, or XVII-XX, wherein R¹ is C₁-C₆ haloalkyl, or a pharmaceutically acceptable salt or solvate thereof.

In another embodiment, Compounds of the Disclosure are compounds of any one of Formulae I-IV, IX-XI, or XVIII-XX, wherein R¹ is optionally substituted C₃-C₈ cycloalkyl, or a pharmaceutically acceptable salt or solvate thereof.

In another embodiment, Compounds of the Disclosure are compounds of any one of Formulae I-IV, IX-XI, or XVIII-XX, wherein R¹ is selected from the group consisting of (hydroxy)alkyl, (amino)alkyl, (alkoxy)alkyl, (cycloalkyl)alkyl, (heterocyclo)alkyl, (heteroaryl)alkyl, and aralkyl, or a pharmaceutically acceptable salt or solvate thereof. In another embodiment, R¹ is (heterocyclo)alkyl, e.g.,

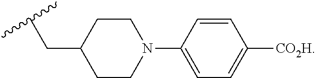 [SMILES: *CC1CCN(c2ccc(C(=O)O)cc2)CC1]

In another embodiment, Compounds of the Disclosure are compounds of any one of Formulae I-IV, IX-XI, or XVIII-XX, wherein R¹ is (hydroxy)alkyl, or a pharmaceutically acceptable salt or solvate thereof.

In another embodiment, Compounds of the Disclosure are compounds of any one of Formulae I-IV, IX-XI, or XVIII-XX, wherein R¹ is (amino)alkyl, or a pharmaceutically acceptable salt or solvate thereof.

In another embodiment, Compounds of the Disclosure are compounds of any one of Formulae I-IV, IX-XI, or XVIII-XX, wherein R¹ is (alkoxy)alkyl, or a pharmaceutically acceptable salt or solvate thereof.

In another embodiment, Compounds of the Disclosure are compounds of any one of Formulae I-IV or IX-XI, w I-IV, IX-XI, or XVIII-XX, herein R¹ is (cycloalkyl)alkyl, or a pharmaceutically acceptable salt or solvate thereof.

In another embodiment, Compounds of the Disclosure are compounds of any one of Formulae I-IV, IX-XI, or XVIII-XX, wherein R¹ is (heterocyclo)alkyl, or a pharmaceutically acceptable salt or solvate thereof.

In another embodiment, Compounds of the Disclosure are compounds of any one of Formulae I-IV, IX-XI, or XVIII-XX, wherein R¹ is (heteroaryl)alkyl, or a pharmaceutically acceptable salt or solvate thereof.

In another embodiment, Compounds of the Disclosure are compounds of any one of Formulae I-IV, IX-XI, or XVIII-XX, wherein R¹ is aralkyl, or a pharmaceutically acceptable salt or solvate thereof.

In another embodiment, Compounds of the Disclosure are compounds of any one of Formulae I-IV, IX-XI, or XVIII-XX, wherein R¹ is selected from the group consisting of optionally substituted 4- to 8-membered heterocyclo, optionally substituted aryl, and optionally substituted heteroaryl, or a pharmaceutically acceptable salt or solvate thereof.

In another embodiment, Compounds of the Disclosure are compounds of any one of Formulae I-IV, IX-XI, or XVIII-XX, wherein R¹ is selected from the group consisting of optionally substituted 4- to 8-membered heterocyclo. In another embodiment, R¹ is optionally substituted 4-membered heterocyclo. In another embodiment, R¹ is optionally substituted 5-membered heterocyclo. In another embodiment, R¹ is optionally substituted 6-membered heterocyclo, e.g.,

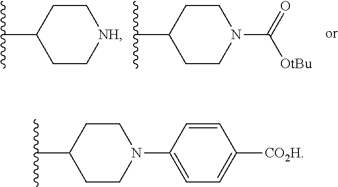

In another embodiment, Compounds of the Disclosure are compounds of any one of Formulae I-IV, IX-XI, or XVIII-XX, wherein R¹ is optionally substituted 4- to 8-membered heterocyclo, or a pharmaceutically acceptable salt or solvate thereof.

In another embodiment, Compounds of the Disclosure are compounds of any one of Formulae I-IV, IX-XI, or XVIII-XX, wherein R¹ is optionally substituted aryl, or a pharmaceutically acceptable salt or solvate thereof.

In another embodiment, Compounds of the Disclosure are compounds of any one of Formulae I-IV, IX-XI, or XVIII-XX, wherein R¹ is optionally substituted heteroaryl, or a pharmaceutically acceptable salt or solvate thereof.

In another embodiment, Compounds of the Disclosure are compounds of any one of Formulae I-IV, IX-XI, or XVIII-XX, wherein R¹ is —C(═O)R⁴, or a pharmaceutically acceptable salt or solvate thereof. In another embodiment, R⁴ is —R^{4a}. In another embodiment, R⁴ is —OR^{4b}. In another embodiment, R^{4b} is C₁-C₆ alkyl. In another embodiment, R⁴ is —NR^{4c}R^{4d};

In another embodiment, Compounds of the Disclosure are compounds of any one of Formulae I-IV, IX-XI, or XVIII-XX, wherein R¹ is —S(═O)₂R⁵, or a pharmaceutically acceptable salt or solvate thereof. In another embodiment, R⁵ is —R^{5a}. In another embodiment, R^{5a} is C₁-C₆ alkyl. In another embodiment, R⁵ is —NR^{5b}R^{5c}.

In another embodiment, Compounds of the Disclosure are compounds of any one of Formulae I-IV, IX-XI, or XVIII-XX, wherein R¹ is —C(═NR⁶)R⁷, or a pharmaceutically acceptable salt or solvate thereof.

In another embodiment, Compounds of the Disclosure are compounds of

Formula II, wherein R¹, R^{2a}, R^{2d}, R³, m, n, and Z are as defined in Table 1, or a pharmaceutically acceptable salt or solvate thereof.

TABLE 1
II

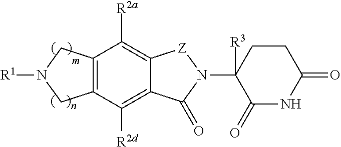

| Cpd. | | | | | | | |
|---|---|---|---|---|---|---|---|
| No. | R¹ | R^{2a} | R^{2d} | R³ | m | n | Z |
| 1 | —H | —H | —H | —H | 1 | 1 | —CH₂— |
| 2 | —CH₃ | —H | —H | —H | 1 | 1 | —CH₂— |
| 3 | —CH₂CH₃ | —H | —H | —H | 1 | 1 | —CH₂— |
| 4 | —CH(CH₃) | —H | —H | —H | 1 | 1 | —CH₂— |
| 5 | —CH₂CH(CH₃) | —H | —H | —H | 1 | 1 | —CH₂— |
| 6 | —C(═O)CH₃ | —H | —H | —H | 1 | 1 | —CH₂— |
| 7 | —C(═O)CF₃ | —H | —H | —H | 1 | 1 | —CH₂— |
| 8 | —C(═O)OCH₃ | —H | —H | —H | 1 | 1 | —CH₂— |
| 9 | —C(═O)OC(CH₃)₃ | —H | —H | —H | 1 | 1 | —CH₂— |
| 10 | —S(═O)₂CH₃ | —H | —H | —H | 1 | 1 | —CH₂— |
| 11 | 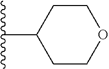 [SMILES: *C1CCOCC1] | —H | —H | —H | 1 | 1 | —CH₂— |
| 12 | 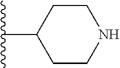 [SMILES: *C1CCNCC1] | —H | —H | —H | 1 | 1 | —CH₂— |
| 13 | 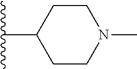 [SMILES: *C1CCN(C)CC1] | —H | —H | —H | 1 | 1 | —CH₂— |
| 14 | 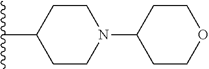 [SMILES: *C1CCN(C2CCOCC2)CC1] | —H | —H | —H | 1 | 1 | —CH₂— |
| 15 | 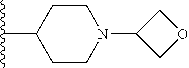 [SMILES: *C1CCN(C2COC2)CC1] | —H | —H | —H | 1 | 1 | —CH₂— |
| 16 | —H | —H | —H | —F | 1 | 1 | —CH₂— |
| 17 | —CH₃ | —H | —H | —F | 1 | 1 | —CH₂— |
| 18 | —CH₂CH₃ | —H | —H | —F | 1 | 1 | —CH₂— |
| 19 | —CH(CH₃) | —H | —H | —F | 1 | 1 | —CH₂— |
| 20 | —CH₂CH(CH₃) | —H | —H | —F | 1 | 1 | —CH₂— |
| 21 | —C(═O)CH₃ | —H | —H | —F | 1 | 1 | —CH₂— |
| 22 | —C(═O)CF₃ | —H | —H | —F | 1 | 1 | —CH₂— |
| 23 | —C(═O)OCH₃ | —H | —H | —F | 1 | 1 | —CH₂— |
| 24 | —C(═O)OC(CH₃)₃ | —H | —H | —F | 1 | 1 | —CH₂— |
| 25 | —S(═O)₂CH₃ | —H | —H | —F | 1 | 1 | —CH₂— |
| 26 | 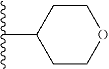 [SMILES: *C1CCOCC1] | —H | —H | —F | 1 | 1 | —CH₂— |
| 27 | 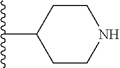 [SMILES: *C1CCNCC1] | —H | —H | —F | 1 | 1 | —CH₂— |
| 28 | 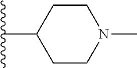 [SMILES: *C1CCN(C)CC1] | —H | —H | —F | 1 | 1 | —CH₂— |
| 29 | 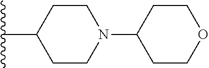 [SMILES: *C1CCN(C2CCOCC2)CC1] | —H | —H | —F | 1 | 1 | —CH₂— |
| 30 | 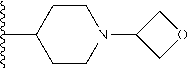 [SMILES: *C1CCN(C2COC2)CC1] | —H | —H | —F | 1 | 1 | —CH₂— |
| 31 | —H | —H | —H | —CH₃ | 1 | 1 | —CH₂— |
| 32 | —CH₃ | —H | —H | —CH₃ | 1 | 1 | —CH₂— |
| 33 | —CH₂CH₃ | —H | —H | —CH₃ | 1 | 1 | —CH₂— |
| 34 | —CH(CH₃) | —H | —H | —CH₃ | 1 | 1 | —CH₂— |
| 35 | —CH₂CH(CH₃) | —H | —H | —CH₃ | 1 | 1 | —CH₂— |
| 36 | —C(═O)CH₃ | —H | —H | —CH₃ | 1 | 1 | —CH₂— |
| 37 | —C(═O)CF₃ | —H | —H | —CH₃ | 1 | 1 | —CH₂— |
| 38 | —C(═O)OCH₃ | —H | —H | —CH₃ | 1 | 1 | —CH₂— |
| 39 | —C(═O)OC(CH₃)₃ | —H | —H | —CH₃ | 1 | 1 | —CH₂— |
| 40 | —S(═O)₂CH₃ | —H | —H | —CH₃ | 1 | 1 | —CH₂— |
| 41 | 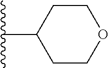 [SMILES: *C1CCOCC1] | —H | —H | —CH₃ | 1 | 1 | —CH₂— |
| 42 | 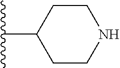 [SMILES: *C1CCNCC1] | —H | —H | —CH₃ | 1 | 1 | —CH₂— |
| 43 | 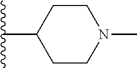 [SMILES: *C1CCN(C)CC1] | —H | —H | —CH₃ | 1 | 1 | —CH₂— |
| 44 | 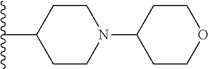 [SMILES: *C1CCN(C2CCOCC2)CC1] | —H | —H | —CH₃ | 1 | 1 | —CH₂— |
| 45 | 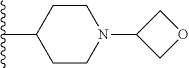 [SMILES: *C1CCN(C2COC2)CC1] | —H | —H | —CH₃ | 1 | 1 | —CH₂— |
| 46 | —H | —H | —H | —D | 1 | 1 | —CH₂— |
| 47 | —CH₃ | —H | —H | —D | 1 | 1 | —CH₂— |
| 48 | —CH₂CH₃ | —H | —H | —D | 1 | 1 | —CH₂— |
| 49 | —CH(CH₃) | —H | —H | —D | 1 | 1 | —CH₂— |
| 50 | —CH₂CH(CH₃) | —H | —H | —D | 1 | 1 | —CH₂— |
| 51 | —C(═O)CH₃ | —H | —H | —D | 1 | 1 | —CH₂— |
| 52 | —C(═O)CF₃ | —H | —H | —D | 1 | 1 | —CH₂— |
| 53 | —C(═O)OCH₃ | —H | —H | —D | 1 | 1 | —CH₂— |
| 54 | —C(═O)OC(CH₃)₃ | —H | —H | —D | 1 | 1 | —CH₂— |
| 55 | —S(═O)₂CH₃ | —H | —H | —D | 1 | 1 | —CH₂— |
| 56 | 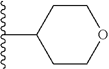 [SMILES: *C1CCOCC1] | —H | —H | —D | 1 | 1 | —CH₂— |
| 57 | 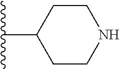 [SMILES: *C1CCNCC1] | —H | —H | —D | 1 | 1 | —CH₂— |
| 58 | 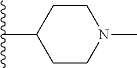 [SMILES: *C1CCN(C)CC1] | —H | —H | —D | 1 | 1 | —CH₂— |
| 59 | 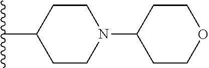 [SMILES: *C1CCN(C2CCOCC2)CC1] | —H | —H | —D | 1 | 1 | —CH₂— |
| 60 | 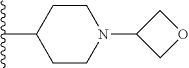 [SMILES: *C1CCN(C2COC2)CC1] | —H | —H | —D | 1 | 1 | —CH₂— |
| 61 | —H | —H | —H | —H | 2 | 1 | —CH₂— |
| 62 | —CH₃ | —H | —H | —H | 2 | 1 | —CH₂— |
| 63 | —CH₂CH₃ | —H | —H | —H | 2 | 1 | —CH₂— |
| 64 | —CH(CH₃) | —H | —H | —H | 2 | 1 | —CH₂— |
| 65 | —CH₂CH(CH₃) | —H | —H | —H | 2 | 1 | —CH₂— |
| 66 | —C(═O)CH₃ | —H | —H | —H | 2 | 1 | —CH₂— |
| 67 | —C(═O)CF₃ | —H | —H | —H | 2 | 1 | —CH₂— |
| 68 | —C(═O)OCH₃ | —H | —H | —H | 2 | 1 | —CH₂— |
| 69 | —C(═O)OC(CH₃)₃ | —H | —H | —H | 2 | 1 | —CH₂— |
| 70 | —S(═O)₂CH₃ | —H | —H | —H | 2 | 1 | —CH₂— |
| 71 | 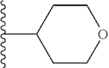 [SMILES: *C1CCOCC1] | —H | —H | —H | 2 | 1 | —CH₂— |
| 72 | 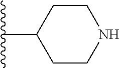 [SMILES: *C1CCNCC1] | —H | —H | —H | 2 | 1 | —CH₂— |
| 73 | 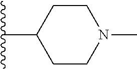 [SMILES: *C1CCN(C)CC1] | —H | —H | —H | 2 | 1 | —CH₂— |
| 74 | 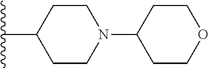 [SMILES: *C1CCN(C2CCOCC2)CC1] | —H | —H | —H | 2 | 1 | —CH₂— |
| 75 | 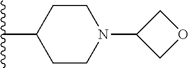 [SMILES: *C1CCN(C2COC2)CC1] | —H | —H | —H | 2 | 1 | —CH₂— |
| 76 | —H | —H | —H | —F | 2 | 1 | —CH₂— |
| 77 | —CH₃ | —H | —H | —F | 2 | 1 | —CH₂— |
| 78 | —CH₂CH₃ | —H | —H | —F | 2 | 1 | —CH₂— |
| 79 | —CH(CH₃) | —H | —H | —F | 2 | 1 | —CH₂— |
| 80 | —CH₂CH(CH₃) | —H | —H | —F | 2 | 1 | —CH₂— |
| 81 | —C(═O)CH₃ | —H | —H | —F | 2 | 1 | —CH₂— |
| 82 | —C(═O)CF₃ | —H | —H | —F | 2 | 1 | —CH₂— |
| 83 | —C(═O)OCH₃ | —H | —H | —F | 2 | 1 | —CH₂— |
| 84 | —C(═O)OC(CH₃)₃ | —H | —H | —F | 2 | 1 | —CH₂— |
| 85 | —S(═O)₂CH₃ | —H | —H | —F | 2 | 1 | —CH₂— |
| 86 | 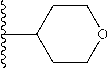 [SMILES: *C1CCOCC1] | —H | —H | —F | 2 | 1 | —CH₂— |
| 87 | 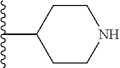 [SMILES: *C1CCNCC1] | —H | —H | —F | 2 | 1 | —CH₂— |
| 88 | 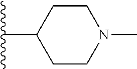 [SMILES: *C1CCN(C)CC1] | —H | —H | —F | 2 | 1 | —CH₂— |
| 89 | 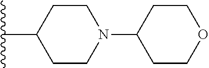 [SMILES: *C1CCN(C2CCOCC2)CC1] | —H | —H | —F | 2 | 1 | —CH₂— |
| 90 | 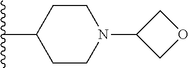 [SMILES: *C1CCN(C2COC2)CC1] | —H | —H | —F | 2 | 1 | —CH₂— |
| 91 | —H | —H | —H | —CH₃ | 2 | 1 | —CH₂— |
| 92 | —CH₃ | —H | —H | —CH₃ | 2 | 1 | —CH₂— |
| 93 | —CH₂CH₃ | —H | —H | —CH₃ | 2 | 1 | —CH₂— |
| 94 | —CH(CH₃) | —H | —H | —CH₃ | 2 | 1 | —CH₂— |
| 95 | —CH₂CH(CH₃) | —H | —H | —CH₃ | 2 | 1 | —CH₂— |
| 96 | —C(═O)CH₃ | —H | —H | —CH₃ | 2 | 1 | —CH₂— |
| 97 | —C(═O)CF₃ | —H | —H | —CH₃ | 2 | 1 | —CH₂— |
| 98 | —C(═O)OCH₃ | —H | —H | —CH₃ | 2 | 1 | —CH₂— |
| 99 | —C(═O)OC(CH₃)₃ | —H | —H | —CH₃ | 2 | 1 | —CH₂— |
| 100 | —S(═O)₂CH₃ | —H | —H | —CH₃ | 2 | 1 | —CH₂— |
| 101 | 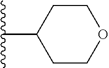 [SMILES: *C1CCOCC1] | —H | —H | —CH₃ | 2 | 1 | —CH₂— |
| 102 | 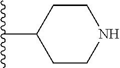 [SMILES: *C1CCNCC1] | —H | —H | —CH₃ | 2 | 1 | —CH₂— |
| 103 | 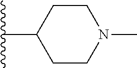 [SMILES: *C1CCN(C)CC1] | —H | —H | —CH₃ | 2 | 1 | —CH₂— |
| 104 | 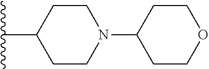 [SMILES: *C1CCN(C2CCOCC2)CC1] | —H | —H | —CH₃ | 2 | 1 | —CH₂— |
| 105 | 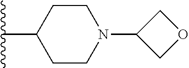 [SMILES: *C1CCN(C2COC2)CC1] | —H | —H | —CH₃ | 2 | 1 | —CH₂— |
| 106 | —H | —H | —H | —D | 2 | 1 | —CH₂— |
| 107 | —CH₃ | —H | —H | —D | 2 | 1 | —CH₂— |
| 108 | —CH₂CH₃ | —H | —H | —D | 2 | 1 | —CH₂— |
| 109 | —CH(CH₃) | —H | —H | —D | 2 | 1 | —CH₂— |
| 110 | —CH₂CH(CH₃) | —H | —H | —D | 2 | 1 | —CH₂— |
| 111 | —C(═O)CH₃ | —H | —H | —D | 2 | 1 | —CH₂— |
| 112 | —C(═O)CF₃ | —H | —H | —D | 2 | 1 | —CH₂— |
| 113 | —C(═O)OCH₃ | —H | —H | —D | 2 | 1 | —CH₂— |
| 114 | —C(═O)OC(CH₃)₃ | —H | —H | —D | 2 | 1 | —CH₂— |
| 115 | —S(═O)₂CH₃ | —H | —H | —D | 2 | 1 | —CH₂— |
| 116 | 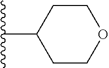 [SMILES: *C1CCOCC1] | —H | —H | —D | 2 | 1 | —CH₂— |
| 117 | 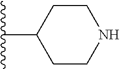 [SMILES: *C1CCNCC1] | —H | —H | —D | 2 | 1 | —CH₂— |
| 118 | 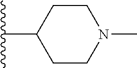 [SMILES: *C1CCN(C)CC1] | —H | —H | —D | 2 | 1 | —CH₂— |
| 119 | 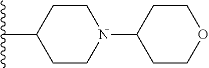 [SMILES: *C1CCN(C2CCOCC2)CC1] | —H | —H | —D | 2 | 1 | —CH₂— |
| 120 | 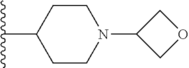 [SMILES: *C1CCN(C2COC2)CC1] | —H | —H | —D | 2 | 1 | —CH₂— |
| 121 | —H | —H | —H | —H | 1 | 2 | —CH₂— |
| 122 | —CH₃ | —H | —H | —H | 1 | 2 | —CH₂— |
| 123 | —CH₂CH₃ | —H | —H | —H | 1 | 2 | —CH₂— |
| 124 | —CH(CH₃) | —H | —H | —H | 1 | 2 | —CH₂— |
| 125 | —CH₂CH(CH₃) | —H | —H | —H | 1 | 2 | —CH₂— |
| 126 | —C(═O)CH₃ | —H | —H | —H | 1 | 2 | —CH₂— |
| 127 | —C(═O)CF₃ | —H | —H | —H | 1 | 2 | —CH₂— |
| 128 | —C(═O)OCH₃ | —H | —H | —H | 1 | 2 | —CH₂— |
| 129 | —C(═O)OC(CH₃)₃ | —H | —H | —H | 1 | 2 | —CH₂— |
| 130 | —S(═O)₂CH₃ | —H | —H | —H | 1 | 2 | —CH₂— |
| 131 | 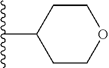 [SMILES: *C1CCOCC1] | —H | —H | —H | 1 | 2 | —CH₂— |
| 132 | 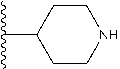 [SMILES: *C1CCNCC1] | —H | —H | —H | 1 | 2 | —CH₂— |
| 133 | 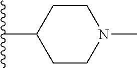 [SMILES: *C1CCN(C)CC1] | —H | —H | —H | 1 | 2 | —CH₂— |
| 134 | 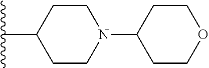 [SMILES: *C1CCN(C2CCOCC2)CC1] | —H | —H | —H | 1 | 2 | —CH₂— |
| 135 | 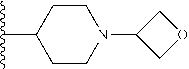 [SMILES: *C1CCN(C2COC2)CC1] | —H | —H | —H | 1 | 2 | —CH₂— |
| 136 | —H | —H | —H | —F | 1 | 2 | —CH₂— |
| 137 | —CH₃ | —H | —H | —F | 1 | 2 | —CH₂— |
| 138 | —CH₂CH₃ | —H | —H | —F | 1 | 2 | —CH₂— |
| 139 | —CH(CH₃) | —H | —H | —F | 1 | 2 | —CH₂— |
| 140 | —CH₂CH(CH₃) | —H | —H | —F | 1 | 2 | —CH₂— |
| 141 | —C(═O)CH₃ | —H | —H | —F | 1 | 2 | —CH₂— |
| 142 | —C(═O)CF₃ | —H | —H | —F | 1 | 2 | —CH₂— |
| 143 | —C(═O)OCH₃ | —H | —H | —F | 1 | 2 | —CH₂— |
| 144 | —C(═O)OC(CH₃)₃ | —H | —H | —F | 1 | 2 | —CH₂— |
| 145 | —S(═O)₂CH₃ | —H | —H | —F | 1 | 2 | —CH₂— |
| 146 | 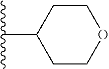 [SMILES: *C1CCOCC1] | —H | —H | —F | 1 | 2 | —CH₂— |
| 147 | 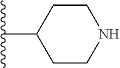 [SMILES: *C1CCNCC1] | —H | —H | —F | 1 | 2 | —CH₂— |
| 148 | 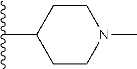 [SMILES: *C1CCN(C)CC1] | —H | —H | —F | 1 | 2 | —CH₂— |
| 149 | 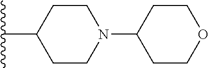 [SMILES: *C1CCN(C2CCOCC2)CC1] | —H | —H | —F | 1 | 2 | —CH₂— |
| 150 | 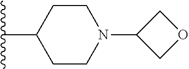 [SMILES: *C1CCN(C2COC2)CC1] | —H | —H | —F | 1 | 2 | —CH₂— |
| 151 | —H | —H | —H | —CH₃ | 1 | 2 | —CH₂— |
| 152 | —CH₃ | —H | —H | —CH₃ | 1 | 2 | —CH₂— |
| 153 | —CH₂CH₃ | —H | —H | —CH₃ | 1 | 2 | —CH₂— |
| 154 | —CH(CH₃) | —H | —H | —CH₃ | 1 | 2 | —CH₂— |
| 155 | —CH₂CH(CH₃) | —H | —H | —CH₃ | 1 | 2 | —CH₂— |
| 156 | —C(═O)CH₃ | —H | —H | —CH₃ | 1 | 2 | —CH₂— |
| 157 | —C(═O)CF₃ | —H | —H | —CH₃ | 1 | 2 | —CH₂— |
| 158 | —C(═O)OCH₃ | —H | —H | —CH₃ | 1 | 2 | —CH₂— |
| 159 | —C(═O)OC(CH₃)₃ | —H | —H | —CH₃ | 1 | 2 | —CH₂— |
| 160 | —S(═O)₂CH₃ | —H | —H | —CH₃ | 1 | 2 | —CH₂— |
| 161 | 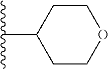 [SMILES: *C1CCOCC1] | —H | —H | —CH₃ | 1 | 2 | —CH₂— |
| 162 | 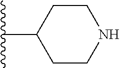 [SMILES: *C1CCNCC1] | —H | —H | —CH₃ | 1 | 2 | —CH₂— |
| 163 | 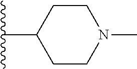 [SMILES: *C1CCN(C)CC1] | —H | —H | —CH₃ | 1 | 2 | —CH₂— |
| 164 | 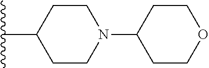 [SMILES: *C1CCN(C2CCOCC2)CC1] | —H | —H | —CH₃ | 1 | 2 | —CH₂— |
| 165 | 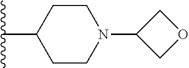 [SMILES: *C1CCN(C2COC2)CC1] | —H | —H | —CH₃ | 1 | 2 | —CH₂— |
| 166 | —H | —H | —H | —D | 1 | 2 | —CH₂— |
| 167 | —CH₃ | —H | —H | —D | 1 | 2 | —CH₂— |
| 168 | —CH₂CH₃ | —H | —H | —D | 1 | 2 | —CH₂— |
| 169 | —CH(CH₃) | —H | —H | —D | 1 | 2 | —CH₂— |
| 170 | —CH₂CH(CH₃) | —H | —H | —D | 1 | 2 | —CH₂— |
| 171 | —C(═O)CH₃ | —H | —H | —D | 1 | 2 | —CH₂— |
| 172 | —C(═O)CF₃ | —H | —H | —D | 1 | 2 | —CH₂— |
| 173 | —C(═O)OCH₃ | —H | —H | —D | 1 | 2 | —CH₂— |
| 174 | —C(═O)OC(CH₃)₃ | —H | —H | —D | 1 | 2 | —CH₂— |
| 175 | —S(═O)₂CH₃ | —H | —H | —D | 1 | 2 | —CH₂— |
| 176 | 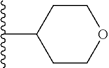 [SMILES: *C1CCOCC1] | —H | —H | —D | 1 | 2 | —CH₂— |
| 177 | 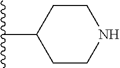 [SMILES: *C1CCNCC1] | —H | —H | —D | 1 | 2 | —CH₂— |
| 178 | 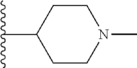 [SMILES: *C1CCN(C)CC1] | —H | —H | —D | 1 | 2 | —CH₂— |
| 179 | 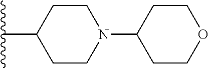 [SMILES: *C1CCN(C2CCOCC2)CC1] | —H | —H | —D | 1 | 2 | —CH₂— |
| 180 | 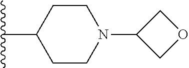 [SMILES: *C1CCN(C2COC2)CC1] | —H | —H | —D | 1 | 2 | —CH₂— |
| 181 | —H | —H | —H | —H | 1 | 1 | —C(═O)— |
| 182 | —CH₃ | —H | —H | —H | 1 | 1 | —C(═O)— |
| 183 | —CH₂CH₃ | —H | —H | —H | 1 | 1 | —C(═O)— |
| 184 | —CH(CH₃) | —H | —H | —H | 1 | 1 | —C(═O)— |
| 185 | —CH₂CH(CH₃) | —H | —H | —H | 1 | 1 | —C(═O)— |
| 186 | —C(═O)CH₃ | —H | —H | —H | 1 | 1 | —C(═O)— |
| 187 | —C(═O)CF₃ | —H | —H | —H | 1 | 1 | —C(═O)— |
| 188 | —C(═O)OCH₃ | —H | —H | —H | 1 | 1 | —C(═O)— |
| 189 | —C(═O)OC(CH₃)₃ | —H | —H | —H | 1 | 1 | —C(═O)— |
| 190 | —S(═O)₂CH₃ | —H | —H | —H | 1 | 1 | —C(═O)— |
| 191 | 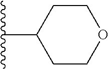 [SMILES: *C1CCOCC1] | —H | —H | —H | 1 | 1 | —C(═O)— |
| 192 | 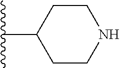 [SMILES: *C1CCNCC1] | —H | —H | —H | 1 | 1 | —C(═O)— |
| 193 | 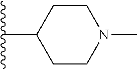 [SMILES: *C1CCN(C)CC1] | —H | —H | —H | 1 | 1 | —C(═O)— |
| 194 | 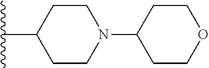 [SMILES: *C1CCN(C2CCOCC2)CC1] | —H | —H | —H | 1 | 1 | —C(═O)— |
| 195 | 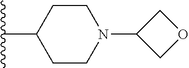 [SMILES: *C1CCN(C2COC2)CC1] | —H | —H | —H | 1 | 1 | —C(═O)— |
| 196 | —H | —H | —H | —F | 1 | 1 | —C(═O)— |
| 197 | —CH₃ | —H | —H | —F | 1 | 1 | —C(═O)— |
| 198 | —CH₂CH₃ | —H | —H | —F | 1 | 1 | —C(═O)— |
| 199 | —CH(CH₃) | —H | —H | —F | 1 | 1 | —C(═O)— |
| 200 | —CH₂CH(CH₃) | —H | —H | —F | 1 | 1 | —C(═O)— |
| 201 | —C(═O)CH₃ | —H | —H | —F | 1 | 1 | —C(═O)— |
| 202 | —C(═O)CF₃ | —H | —H | —F | 1 | 1 | —C(═O)— |
| 203 | —C(═O)OCH₃ | —H | —H | —F | 1 | 1 | —C(═O)— |
| 204 | —C(═O)OC(CH₃)₃ | —H | —H | —F | 1 | 1 | —C(═O)— |
| 205 | —S(═O)₂CH₃ | —H | —H | —F | 1 | 1 | —C(═O)— |
| 206 | 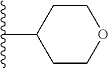 [SMILES: *C1CCOCC1] | —H | —H | —F | 1 | 1 | —C(═O)— |
| 207 | 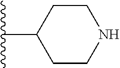 [SMILES: *C1CCNCC1] | —H | —H | —F | 1 | 1 | —C(═O)— |
| 208 | 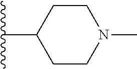 [SMILES: *C1CCN(C)CC1] | —H | —H | —F | 1 | 1 | —C(═O)— |
| 209 | 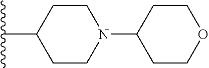 [SMILES: *C1CCN(C2CCOCC2)CC1] | —H | —H | —F | 1 | 1 | —C(═O)— |
| 210 | 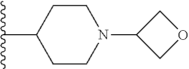 [SMILES: *C1CCN(C2COC2)CC1] | —H | —H | —F | 1 | 1 | —C(═O)— |
| 211 | —H | —H | —H | —CH₃ | 1 | 1 | —C(═O)— |
| 212 | —CH₃ | —H | —H | —CH₃ | 1 | 1 | —C(═O)— |
| 213 | —CH₂CH₃ | —H | —H | —CH₃ | 1 | 1 | —C(═O)— |
| 214 | —CH(CH₃) | —H | —H | —CH₃ | 1 | 1 | —C(═O)— |
| 215 | —CH₂CH(CH₃) | —H | —H | —CH₃ | 1 | 1 | —C(═O)— |
| 216 | —C(═O)CH₃ | —H | —H | —CH₃ | 1 | 1 | —C(═O)— |
| 217 | —C(═O)CF₃ | —H | —H | —CH₃ | 1 | 1 | —C(═O)— |
| 218 | —C(═O)OCH₃ | —H | —H | —CH₃ | 1 | 1 | —C(═O)— |
| 219 | —C(═O)OC(CH₃)₃ | —H | —H | —CH₃ | 1 | 1 | —C(═O)— |
| 220 | —S(═O)₂CH₃ | —H | —H | —CH₃ | 1 | 1 | —C(═O)— |
| 221 | 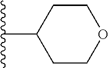 [SMILES: *C1CCOCC1] | —H | —H | —CH₃ | 1 | 1 | —C(═O)— |
| 222 | 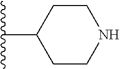 [SMILES: *C1CCNCC1] | —H | —H | —CH₃ | 1 | 1 | —C(═O)— |
| 223 | 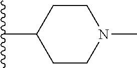 [SMILES: *C1CCN(C)CC1] | —H | —H | —CH₃ | 1 | 1 | —C(═O)— |
| 224 | 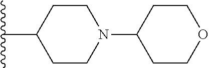 [SMILES: *C1CCN(C2CCOCC2)CC1] | —H | —H | —CH₃ | 1 | 1 | —C(═O)— |
| 225 | 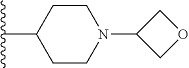 [SMILES: *C1CCN(C2COC2)CC1] | —H | —H | —CH₃ | 1 | 1 | —C(═O)— |
| 226 | —H | —H | —H | —D | 1 | 1 | —C(═O)— |
| 227 | —CH₃ | —H | —H | —D | 1 | 1 | —C(═O)— |
| 228 | —CH₂CH₃ | —H | —H | —D | 1 | 1 | —C(═O)— |
| 229 | —CH(CH₃) | —H | —H | —D | 1 | 1 | —C(═O)— |
| 230 | —CH₂CH(CH₃) | —H | —H | —D | 1 | 1 | —C(═O)— |
| 231 | —C(═O)CH₃ | —H | —H | —D | 1 | 1 | —C(═O)— |
| 232 | —C(═O)CF₃ | —H | —H | —D | 1 | 1 | —C(═O)— |
| 233 | —C(═O)OCH₃ | —H | —H | —D | 1 | 1 | —C(═O)— |
| 234 | —C(═O)OC(CH₃)₃ | —H | —H | —D | 1 | 1 | —C(═O)— |
| 235 | —S(═O)₂CH₃ | —H | —H | —D | 1 | 1 | —C(═O)— |
| 236 | 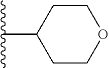 [SMILES: *C1CCOCC1] | —H | —H | —D | 1 | 1 | —C(═O)— |
| 237 | 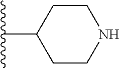 [SMILES: *C1CCNCC1] | —H | —H | —D | 1 | 1 | —C(═O)— |
| 238 | 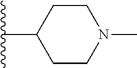 [SMILES: *C1CCN(C)CC1] | —H | —H | —D | 1 | 1 | —C(═O)— |
| 239 | 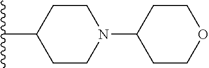 [SMILES: *C1CCN(C2CCOCC2)CC1] | —H | —H | —D | 1 | 1 | —C(═O)— |
| 240 | 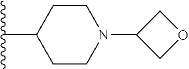 [SMILES: *C1CCN(C2COC2)CC1] | —H | —H | —D | 1 | 1 | —C(═O)— |
| 241 | —H | —H | —H | —H | 2 | 1 | —C(═O)— |
| 242 | —CH₃ | —H | —H | —H | 2 | 1 | —C(═O)— |
| 243 | —CH₂CH₃ | —H | —H | —H | 2 | 1 | —C(═O)— |
| 244 | —CH(CH₃) | —H | —H | —H | 2 | 1 | —C(═O)— |
| 245 | —CH₂CH(CH₃) | —H | —H | —H | 2 | 1 | —C(═O)— |
| 246 | —C(═O)CH₃ | —H | —H | —H | 2 | 1 | —C(═O)— |
| 247 | —C(═O)CF₃ | —H | —H | —H | 2 | 1 | —C(═O)— |
| 248 | —C(═O)OCH₃ | —H | —H | —H | 2 | 1 | —C(═O)— |
| 249 | —C(═O)OC(CH₃)₃ | —H | —H | —H | 2 | 1 | —C(═O)— |
| 250 | —S(═O)₂CH₃ | —H | —H | —H | 2 | 1 | —C(═O)— |
| 251 | 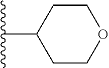 [SMILES: *C1CCOCC1] | —H | —H | —H | 2 | 1 | —C(═O)— |
| 252 | 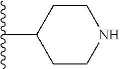 [SMILES: *C1CCNCC1] | —H | —H | —H | 2 | 1 | —C(═O)— |
| 253 | 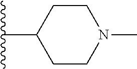 [SMILES: *C1CCN(C)CC1] | —H | —H | —H | 2 | 1 | —C(═O)— |
| 254 | 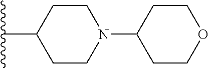 [SMILES: *C1CCN(C2CCOCC2)CC1] | —H | —H | —H | 2 | 1 | —C(═O)— |
| 255 | 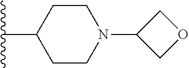 [SMILES: *C1CCN(C2COC2)CC1] | —H | —H | —H | 2 | 1 | —C(═O)— |
| 256 | —H | —H | —H | —F | 2 | 1 | —C(═O)— |
| 257 | —CH₃ | —H | —H | —F | 2 | 1 | —C(═O)— |
| 258 | —CH₂CH₃ | —H | —H | —F | 2 | 1 | —C(═O)— |
| 259 | —CH(CH₃) | —H | —H | —F | 2 | 1 | —C(═O)— |
| 260 | —CH₂CH(CH₃) | —H | —H | —F | 2 | 1 | —C(═O)— |
| 261 | —C(═O)CH₃ | —H | —H | —F | 2 | 1 | —C(═O)— |
| 262 | —C(═O)CF₃ | —H | —H | —F | 2 | 1 | —C(═O)— |
| 263 | —C(═O)OCH₃ | —H | —H | —F | 2 | 1 | —C(═O)— |
| 264 | —C(═O)OC(CH₃)₃ | —H | —H | —F | 2 | 1 | —C(═O)— |
| 265 | —S(═O)₂CH₃ | —H | —H | —F | 2 | 1 | —C(═O)— |
| 266 | 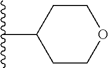 [SMILES: *C1CCOCC1] | —H | —H | —F | 2 | 1 | —C(═O)— |
| 267 | 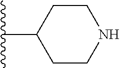 [SMILES: *C1CCNCC1] | —H | —H | —F | 2 | 1 | —C(═O)— |
| 268 | 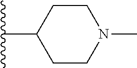 [SMILES: *C1CCN(C)CC1] | —H | —H | —F | 2 | 1 | —C(═O)— |
| 269 | 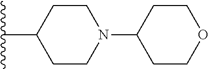 [SMILES: *C1CCN(C2CCOCC2)CC1] | —H | —H | —F | 2 | 1 | —C(═O)— |
| 270 | 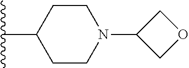 [SMILES: *C1CCN(C2COC2)CC1] | —H | —H | —F | 2 | 1 | —C(═O)— |
| 271 | —H | —H | —H | —CH₃ | 2 | 1 | —C(═O)— |
| 272 | —CH₃ | —H | —H | —CH₃ | 2 | 1 | —C(═O)— |
| 273 | —CH₂CH₃ | —H | —H | —CH₃ | 2 | 1 | —C(═O)— |
| 274 | —CH(CH₃) | —H | —H | —CH₃ | 2 | 1 | —C(═O)— |
| 275 | —CH₂CH(CH₃) | —H | —H | —CH₃ | 2 | 1 | —C(═O)— |
| 276 | —C(═O)CH₃ | —H | —H | —CH₃ | 2 | 1 | —C(═O)— |
| 277 | —C(═O)CF₃ | —H | —H | —CH₃ | 2 | 1 | —C(═O)— |
| 278 | —C(═O)OCH₃ | —H | —H | —CH₃ | 2 | 1 | —C(═O)— |
| 279 | —C(═O)OC(CH₃)₃ | —H | —H | —CH₃ | 2 | 1 | —C(═O)— |
| 280 | —S(═O)₂CH₃ | —H | —H | —CH₃ | 2 | 1 | —C(═O)— |
| 281 | 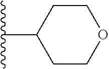 [SMILES: *C1CCOCC1] | —H | —H | —CH₃ | 2 | 1 | —C(═O)— |
| 282 | 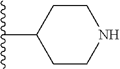 [SMILES: *C1CCNCC1] | —H | —H | —CH₃ | 2 | 1 | —C(═O)— |
| 283 | 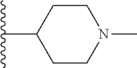 [SMILES: *C1CCN(C)CC1] | —H | —H | —CH₃ | 2 | 1 | —C(═O)— |
| 284 | 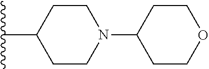 [SMILES: *C1CCN(C2CCOCC2)CC1] | —H | —H | —CH₃ | 2 | 1 | —C(═O)— |
| 285 | 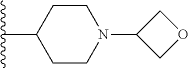 [SMILES: *C1CCN(C2COC2)CC1] | —H | —H | —CH₃ | 2 | 1 | —C(═O)— |
| 286 | —H | —H | —H | —D | 2 | 1 | —C(═O)— |
| 287 | —CH₃ | —H | —H | —D | 2 | 1 | —C(═O)— |
| 288 | —CH₂CH₃ | —H | —H | —D | 2 | 1 | —C(═O)— |
| 289 | —CH(CH₃) | —H | —H | —D | 2 | 1 | —C(═O)— |
| 290 | —CH₂CH(CH₃) | —H | —H | —D | 2 | 1 | —C(═O)— |
| 291 | —C(═O)CH₃ | —H | —H | —D | 2 | 1 | —C(═O)— |
| 292 | —C(═O)CF₃ | —H | —H | —D | 2 | 1 | —C(═O)— |
| 293 | —C(═O)OCH₃ | —H | —H | —D | 2 | 1 | —C(═O)— |
| 294 | —C(═O)OC(CH₃)₃ | —H | —H | —D | 2 | 1 | —C(═O)— |
| 295 | —S(═O)₂CH₃ | —H | —H | —D | 2 | 1 | —C(═O)— |
| 296 | 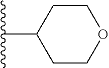 [SMILES: *C1CCOCC1] | —H | —H | —D | 2 | 1 | —C(═O)— |
| 297 | 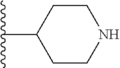 [SMILES: *C1CCNCC1] | —H | —H | —D | 2 | 1 | —C(═O)— |
| 298 | 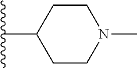 [SMILES: *C1CCN(C)CC1] | —H | —H | —D | 2 | 1 | —C(═O)— |
| 299 | 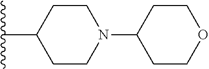 [SMILES: *C1CCN(C2CCOCC2)CC1] | —H | —H | —D | 2 | 1 | —C(═O)— |
| 300 | 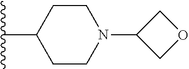 [SMILES: *C1CCN(C2COC2)CC1] | —H | —H | —D | 2 | 1 | —C(═O)— |
| 828 | 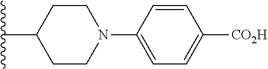 [SMILES: *C1CCN(c2ccc(C(=O)O)cc2)CC1] | —H | —H | —H | 1 | 1 | —C(═O)— |
| 829 | 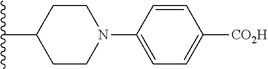 [SMILES: *C1CCN(c2ccc(C(=O)O)cc2)CC1] | —H | —H | —H | 1 | 1 | —CH₂— |
| 830 | 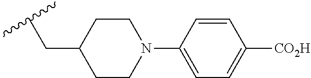 [SMILES: *CC1CCN(c2ccc(C(=O)O)cc2)CC1] | —H | —H | —H | 1 | 1 | —C(═O)— |
| 831 | 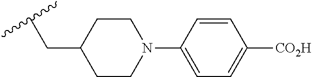 [SMILES: *CC1CCN(c2ccc(C(=O)O)cc2)CC1] | —H | —H | —H | 1 | 1 | —CH₂— |
| 832 | 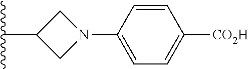 [SMILES: *C1CN(c2ccc(C(=O)O)cc2)C1] | —H | —H | —H | 1 | 1 | —C(═O)— |
| 833 | 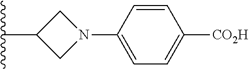 [SMILES: *C1CN(c2ccc(C(=O)O)cc2)C1] | —H | —H | —H | 1 | 1 | —CH₂— |
| 834 | —CH₂C(═O)OH | —H | —H | —H | 1 | 1 | —C(═O)— |
| 835 | —CH₂C(═O)OH | —H | —H | —H | 1 | 1 | —CH₂— |
| 836 | 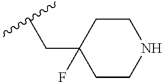 [SMILES: *CC1(F)CCNCC1] | —H | —H | —H | 1 | 1 | —C(═O)— |
| 837 | 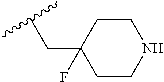 [SMILES: *CC1(F)CCNCC1] | —H | —H | —H | 1 | 1 | —CH₂— |
| 838 | 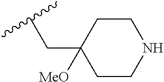 [SMILES: *CC1(OC)CCNCC1] | —H | —H | —H | 1 | 1 | —C(═O)— |
| 839 | 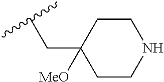 [SMILES: *CC1(OC)CCNCC1] | —H | —H | —H | 1 | 1 | —CH₂— |
| 840 | 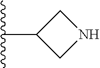 [SMILES: *C1CNC1] | —H | —H | —H | 1 | 1 | —C(═O)— |
| 841 | 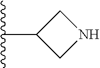 [SMILES: *C1CNC1] | —H | —H | —H | 1 | 1 | —CH₂— |
| 842 | 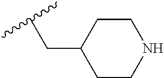 [SMILES: *CC1CCNCC1] | —H | —H | —H | 1 | 1 | —C(═O)— |
| 843 | 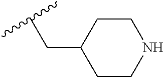 [SMILES: *CC1CCNCC1] | —H | —H | —H | 1 | 1 | —CH₂— |
| 842 | 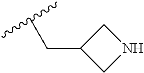 [SMILES: *CC1CNC1] | —H | —H | —H | 1 | 1 | —C(═O)— |
| 843 | 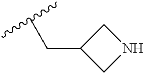 [SMILES: *CC1CNC1] | —H | —H | —H | 1 | 1 | —C(═O)— |
| 854 | —C(═O)OC(CH₃)₃ | —H | —H | —H | 2 | 2 | —C(═O)— |
| 855 | —H | —H | —H | —H | 2 | 2 | —C(═O)— |
| 856 | —C(═O)OC(CH₃)₃ | —H | —H | —H | 1 | 3 | —C(═O)— |
| 857 | —H | —H | —H | —H | 1 | 3 | —C(═O)— |

In another embodiment, Compounds of the Disclosure are compounds of Formula III, wherein R¹, R^{2c},, R^{2d}, R³, m, n, and Z are as defined in Table 2, or a pharmaceutically acceptable salt or solvate thereof.

TABLE 2
III

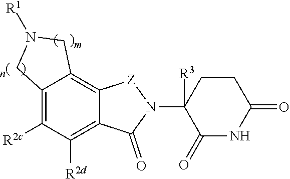

| Cpd. No. | R¹ | R^{2a} | R^{2d} | R³ | m | n | Z |
|---|---|---|---|---|---|---|---|
| 301 | —H | —H | —H | —H | 1 | 1 | —CH₂— |
| 302 | —CH₃ | —H | —H | —H | 1 | 1 | —CH₂— |
| 303 | —CH₂CH₃ | —H | —H | —H | 1 | 1 | —CH₂— |
| 304 | —CH(CH₃) | —H | —H | —H | 1 | 1 | —CH₂— |
| 305 | —CH₂CH(CH₃) | —H | —H | —H | 1 | 1 | —CH₂— |
| 306 | —C(═O)CH₃ | —H | —H | —H | 1 | 1 | —CH₂— |
| 307 | —C(═O)CF₃ | —H | —H | —H | 1 | 1 | —CH₂— |
| 308 | —C(═O)OCH₃ | —H | —H | —H | 1 | 1 | —CH₂— |
| 309 | —C(═O)OC(CH₃)₃ | —H | —H | —H | 1 | 1 | —CH₂— |
| 310 | —S(═O)₂CH₃ | —H | —H | —H | 1 | 1 | —CH₂— |
| 311 | 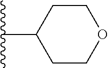 [SMILES: *C1CCOCC1] | —H | —H | —H | 1 | 1 | —CH₂— |
| 312 | 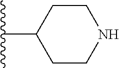 [SMILES: *C1CCNCC1] | —H | —H | —H | 1 | 1 | —CH₂— |
| 313 | 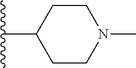 [SMILES: *C1CCN(C)CC1] | —H | —H | —H | 1 | 1 | —CH₂— |
| 314 | 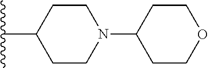 [SMILES: *C1CCN(C2CCOCC2)CC1] | —H | —H | —H | 1 | 1 | —CH₂— |
| 315 | 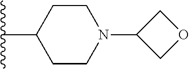 [SMILES: *C1CCN(C2COC2)CC1] | —H | —H | —H | 1 | 1 | —CH₂— |
| 316 | —H | —H | —H | —F | 1 | 1 | —CH₂— |
| 317 | —CH₃ | —H | —H | —F | 1 | 1 | —CH₂— |
| 318 | —CH₂CH₃ | —H | —H | —F | 1 | 1 | —CH₂— |
| 319 | —CH(CH₃) | —H | —H | —F | 1 | 1 | —CH₂— |
| 320 | —CH₂CH(CH₃) | —H | —H | —F | 1 | 1 | —CH₂— |
| 321 | —C(═O)CH₃ | —H | —H | —F | 1 | 1 | —CH₂— |
| 322 | —C(═O)CF₃ | —H | —H | —F | 1 | 1 | —CH₂— |
| 323 | —C(═O)OCH₃ | —H | —H | —F | 1 | 1 | —CH₂— |
| 324 | —C(═O)OC(CH₃)₃ | —H | —H | —F | 1 | 1 | —CH₂— |
| 325 | —S(═O)₂CH₃ | —H | —H | —F | 1 | 1 | —CH₂— |
| 326 | 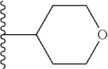 [SMILES: *C1CCOCC1] | —H | —H | —F | 1 | 1 | —CH₂— |
| 327 | 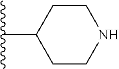 [SMILES: *C1CCNCC1] | —H | —H | —F | 1 | 1 | —CH₂— |
| 328 | 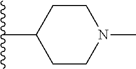 [SMILES: *C1CCN(C)CC1] | —H | —H | —F | 1 | 1 | —CH₂— |
| 329 | 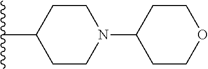 [SMILES: *C1CCN(C2CCOCC2)CC1] | —H | —H | —F | 1 | 1 | —CH₂— |
| 330 | 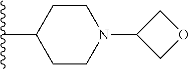 [SMILES: *C1CCN(C2COC2)CC1] | —H | —H | —F | 1 | 1 | —CH₂— |
| 331 | —H | —H | —H | —CH₃ | 1 | 1 | —CH₂— |
| 332 | —CH₃ | —H | —H | —CH₃ | 1 | 1 | —CH₂— |
| 333 | —CH₂CH₃ | —H | —H | —CH₃ | 1 | 1 | —CH₂— |
| 334 | —CH(CH₃) | —H | —H | —CH₃ | 1 | 1 | —CH₂— |
| 335 | —CH₂CH(CH₃) | —H | —H | —CH₃ | 1 | 1 | —CH₂— |
| 336 | —C(═O)CH₃ | —H | —H | —CH₃ | 1 | 1 | —CH₂— |
| 337 | —C(═O)CF₃ | —H | —H | —CH₃ | 1 | 1 | —CH₂— |
| 338 | —C(═O)OCH₃ | —H | —H | —CH₃ | 1 | 1 | —CH₂— |
| 339 | —C(═O)OC(CH₃)₃ | —H | —H | —CH₃ | 1 | 1 | —CH₂— |
| 340 | —S(═O)₂CH₃ | —H | —H | —CH₃ | 1 | 1 | —CH₂— |
| 341 | 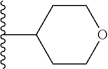 [SMILES: *C1CCOCC1] | —H | —H | —CH₃ | 1 | 1 | —CH₂— |
| 342 | 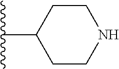 [SMILES: *C1CCNCC1] | —H | —H | —CH₃ | 1 | 1 | —CH₂— |
| 343 | 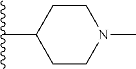 [SMILES: *C1CCN(C)CC1] | —H | —H | —CH₃ | 1 | 1 | —CH₂— |
| 344 | 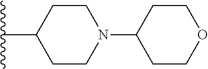 [SMILES: *C1CCN(C2CCOCC2)CC1] | —H | —H | —CH₃ | 1 | 1 | —CH₂— |
| 345 | 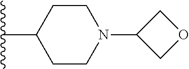 [SMILES: *C1CCN(C2COC2)CC1] | —H | —H | —CH₃ | 1 | 1 | —CH₂— |
| 346 | —H | —H | —H | —D | 1 | 1 | —CH₂— |
| 347 | —CH₃ | —H | —H | —D | 1 | 1 | —CH₂— |
| 348 | —CH₂CH₃ | —H | —H | —D | 1 | 1 | —CH₂— |
| 349 | —CH(CH₃) | —H | —H | —D | 1 | 1 | —CH₂— |
| 350 | —CH₂CH(CH₃) | —H | —H | —D | 1 | 1 | —CH₂— |
| 351 | —C(═O)CH₃ | —H | —H | —D | 1 | 1 | —CH₂— |
| 352 | —C(═O)CF₃ | —H | —H | —D | 1 | 1 | —CH₂— |
| 353 | —C(═O)OCH₃ | —H | —H | —D | 1 | 1 | —CH₂— |
| 354 | —C(═O)OC(CH₃)₃ | —H | —H | —D | 1 | 1 | —CH₂— |
| 355 | —S(═O)₂CH₃ | —H | —H | —D | 1 | 1 | —CH₂— |
| 356 | 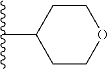 [SMILES: *C1CCOCC1] | —H | —H | —D | 1 | 1 | —CH₂— |
| 357 | 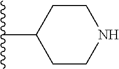 [SMILES: *C1CCNCC1] | —H | —H | —D | 1 | 1 | —CH₂— |
| 358 | 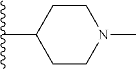 [SMILES: *C1CCN(C)CC1] | —H | —H | —D | 1 | 1 | —CH₂— |
| 359 | 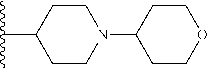 [SMILES: *C1CCN(C2CCOCC2)CC1] | —H | —H | —D | 1 | 1 | —CH₂— |
| 360 | 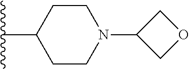 [SMILES: *C1CCN(C2COC2)CC1] | —H | —H | —D | 1 | 1 | —CH₂— |
| 361 | —H | —H | —H | —H | 2 | 1 | —CH₂— |
| 362 | —CH₃ | —H | —H | —H | 2 | 1 | —CH₂— |
| 363 | —CH₂CH₃ | —H | —H | —H | 2 | 1 | —CH₂— |
| 364 | —CH(CH₃) | —H | —H | —H | 2 | 1 | —CH₂— |
| 365 | —CH₂CH(CH₃) | —H | —H | —H | 2 | 1 | —CH₂— |
| 366 | —C(═O)CH₃ | —H | —H | —H | 2 | 1 | —CH₂— |
| 367 | —C(═O)CF₃ | —H | —H | —H | 2 | 1 | —CH₂— |
| 368 | —C(═O)OCH₃ | —H | —H | —H | 2 | 1 | —CH₂— |
| 369 | —C(═O)OC(CH₃)₃ | —H | —H | —H | 2 | 1 | —CH₂— |
| 370 | —S(═O)₂CH₃ | —H | —H | —H | 2 | 1 | —CH₂— |
| 371 | 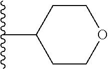 [SMILES: *C1CCOCC1] | —H | —H | —H | 2 | 1 | —CH₂— |
| 372 | 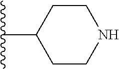 [SMILES: *C1CCNCC1] | —H | —H | —H | 2 | 1 | —CH₂— |
| 373 | 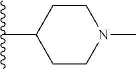 [SMILES: *C1CCN(C)CC1] | —H | —H | —H | 2 | 1 | —CH₂— |
| 374 | 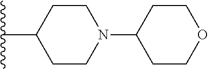 [SMILES: *C1CCN(C2CCOCC2)CC1] | —H | —H | —H | 2 | 1 | —CH₂— |
| 375 | 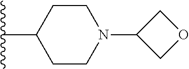 [SMILES: *C1CCN(C2COC2)CC1] | —H | —H | —H | 2 | 1 | —CH₂— |
| 376 | —H | —H | —H | —F | 2 | 1 | —CH₂— |
| 377 | —CH₃ | —H | —H | —F | 2 | 1 | —CH₂— |
| 378 | —CH₂CH₃ | —H | —H | —F | 2 | 1 | —CH₂— |
| 379 | —CH(CH₃) | —H | —H | —F | 2 | 1 | —CH₂— |
| 380 | —CH₂CH(CH₃) | —H | —H | —F | 2 | 1 | —CH₂— |
| 381 | —C(═O)CH₃ | —H | —H | —F | 2 | 1 | —CH₂— |
| 382 | —C(═O)CF₃ | —H | —H | —F | 2 | 1 | —CH₂— |
| 383 | —C(═O)OCH₃ | —H | —H | —F | 2 | 1 | —CH₂— |
| 384 | —C(═O)OC(CH₃)₃ | —H | —H | —F | 2 | 1 | —CH₂— |
| 385 | —S(═O)₂CH₃ | —H | —H | —F | 2 | 1 | —CH₂— |
| 386 | 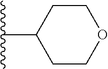 [SMILES: *C1CCOCC1] | —H | —H | —F | 2 | 1 | —CH₂— |
| 387 | 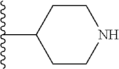 [SMILES: *C1CCNCC1] | —H | —H | —F | 2 | 1 | —CH₂— |
| 388 | 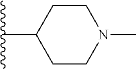 [SMILES: *C1CCN(C)CC1] | —H | —H | —F | 2 | 1 | —CH₂— |
| 389 | 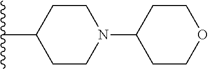 [SMILES: *C1CCN(C2CCOCC2)CC1] | —H | —H | —F | 2 | 1 | —CH₂— |
| 390 | 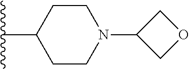 [SMILES: *C1CCN(C2COC2)CC1] | —H | —H | —F | 2 | 1 | —CH₂— |
| 391 | —H | —H | —H | —CH₃ | 2 | 1 | —CH₂— |
| 392 | —CH₃ | —H | —H | —CH₃ | 2 | 1 | —CH₂— |
| 393 | —CH₂CH₃ | —H | —H | —CH₃ | 2 | 1 | —CH₂— |
| 394 | —CH(CH₃) | —H | —H | —CH₃ | 2 | 1 | —CH₂— |
| 395 | —CH₂CH(CH₃) | —H | —H | —CH₃ | 2 | 1 | —CH₂— |
| 396 | —C(═O)CH₃ | —H | —H | —CH₃ | 2 | 1 | —CH₂— |
| 397 | —C(═O)CF₃ | —H | —H | —CH₃ | 2 | 1 | —CH₂— |
| 398 | —C(═O)OCH₃ | —H | —H | —CH₃ | 2 | 1 | —CH₂— |
| 399 | —C(═O)OC(CH₃)₃ | —H | —H | —CH₃ | 2 | 1 | —CH₂— |
| 400 | —S(═O)₂CH₃ | —H | —H | —CH₃ | 2 | 1 | —CH₂— |
| 401 | 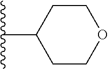 [SMILES: *C1CCOCC1] | —H | —H | —CH₃ | 2 | 1 | —CH₂— |
| 402 | 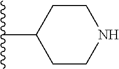 [SMILES: *C1CCNCC1] | —H | —H | —CH₃ | 2 | 1 | —CH₂— |
| 403 | 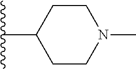 [SMILES: *C1CCN(C)CC1] | —H | —H | —CH₃ | 2 | 1 | —CH₂— |
| 404 | 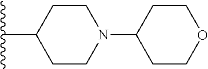 [SMILES: *C1CCN(C2CCOCC2)CC1] | —H | —H | —CH₃ | 2 | 1 | —CH₂— |
| 405 | 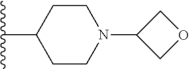 [SMILES: *C1CCN(C2COC2)CC1] | —H | —H | —CH₃ | 2 | 1 | —CH₂— |
| 406 | —H | —H | —H | —D | 2 | 1 | —CH₂— |
| 407 | —CH₃ | —H | —H | —D | 2 | 1 | —CH₂— |
| 408 | —CH₂CH₃ | —H | —H | —D | 2 | 1 | —CH₂— |
| 409 | —CH(CH₃) | —H | —H | —D | 2 | 1 | —CH₂— |
| 410 | —CH₂CH(CH₃) | —H | —H | —D | 2 | 1 | —CH₂— |
| 411 | —C(═O)CH₃ | —H | —H | —D | 2 | 1 | —CH₂— |
| 412 | —C(═O)CF₃ | —H | —H | —D | 2 | 1 | —CH₂— |
| 413 | —C(═O)OCH₃ | —H | —H | —D | 2 | 1 | —CH₂— |
| 414 | —C(═O)OC(CH₃)₃ | —H | —H | —D | 2 | 1 | —CH₂— |
| 415 | —S(═O)₂CH₃ | —H | —H | —D | 2 | 1 | —CH₂— |
| 416 | 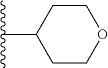 [SMILES: *C1CCOCC1] | —H | —H | —D | 2 | 1 | —CH₂— |
| 417 | 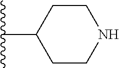 [SMILES: *C1CCNCC1] | —H | —H | —D | 2 | 1 | —CH₂— |
| 418 | 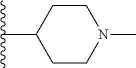 [SMILES: *C1CCN(C)CC1] | —H | —H | —D | 2 | 1 | —CH₂— |
| 419 | 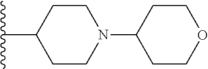 [SMILES: *C1CCN(C2CCOCC2)CC1] | —H | —H | —D | 2 | 1 | —CH₂— |
| 420 | 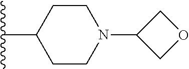 [SMILES: *C1CCN(C2COC2)CC1] | —H | —H | —D | 2 | 1 | —CH₂— |
| 421 | —H | —H | —H | —H | 1 | 2 | —CH₂— |
| 422 | —CH₃ | —H | —H | —H | 1 | 2 | —CH₂— |
| 423 | —CH₂CH₃ | —H | —H | —H | 1 | 2 | —CH₂— |
| 424 | —CH(CH₃) | —H | —H | —H | 1 | 2 | —CH₂— |
| 425 | —CH₂CH(CH₃) | —H | —H | —H | 1 | 2 | —CH₂— |
| 426 | —C(═O)CH₃ | —H | —H | —H | 1 | 2 | —CH₂— |
| 427 | —C(═O)CF₃ | —H | —H | —H | 1 | 2 | —CH₂— |
| 428 | —C(═O)OCH₃ | —H | —H | —H | 1 | 2 | —CH₂— |
| 429 | —C(═O)OC(CH₃)₃ | —H | —H | —H | 1 | 2 | —CH₂— |
| 430 | —S(═O)₂CH₃ | —H | —H | —H | 1 | 2 | —CH₂— |
| 431 | 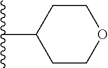 [SMILES: *C1CCOCC1] | —H | —H | —H | 1 | 2 | —CH₂— |
| 432 | 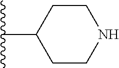 [SMILES: *C1CCNCC1] | —H | —H | —H | 1 | 2 | —CH₂— |
| 433 | 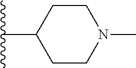 [SMILES: *C1CCN(C)CC1] | —H | —H | —H | 1 | 2 | —CH₂— |
| 434 | 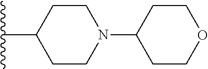 [SMILES: *C1CCN(C2CCOCC2)CC1] | —H | —H | —H | 1 | 2 | —CH₂— |
| 435 | 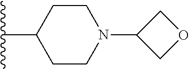 [SMILES: *C1CCN(C2COC2)CC1] | —H | —H | —H | 1 | 2 | —CH₂— |
| 436 | —H | —H | —H | —F | 1 | 2 | —CH₂— |
| 437 | —CH₃ | —H | —H | —F | 1 | 2 | —CH₂— |
| 438 | —CH₂CH₃ | —H | —H | —F | 1 | 2 | —CH₂— |
| 439 | —CH(CH₃) | —H | —H | —F | 1 | 2 | —CH₂— |
| 440 | —CH₂CH(CH₃) | —H | —H | —F | 1 | 2 | —CH₂— |
| 441 | —C(═O)CH₃ | —H | —H | —F | 1 | 2 | —CH₂— |
| 442 | —C(═O)CF₃ | —H | —H | —F | 1 | 2 | —CH₂— |
| 443 | —C(═O)OCH₃ | —H | —H | —F | 1 | 2 | —CH₂— |
| 444 | —C(═O)OC(CH₃)₃ | —H | —H | —F | 1 | 2 | —CH₂— |
| 445 | —S(═O)₂CH₃ | —H | —H | —F | 1 | 2 | —CH₂— |
| 446 | 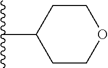 [SMILES: *C1CCOCC1] | —H | —H | —F | 1 | 2 | —CH₂— |
| 447 | 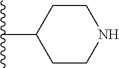 [SMILES: *C1CCNCC1] | —H | —H | —F | 1 | 2 | —CH₂— |
| 448 | 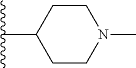 [SMILES: *C1CCN(C)CC1] | —H | —H | —F | 1 | 2 | —CH₂— |
| 449 | 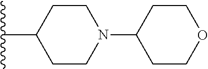 [SMILES: *C1CCN(C2CCOCC2)CC1] | —H | —H | —F | 1 | 2 | —CH₂— |
| 450 | 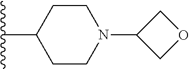 [SMILES: *C1CCN(C2COC2)CC1] | —H | —H | —F | 1 | 2 | —CH₂— |
| 451 | —H | —H | —H | —CH₃ | 1 | 2 | —CH₂— |
| 452 | —CH₃ | —H | —H | —CH₃ | 1 | 2 | —CH₂— |
| 453 | —CH₂CH₃ | —H | —H | —CH₃ | 1 | 2 | —CH₂— |
| 454 | —CH(CH₃) | —H | —H | —CH₃ | 1 | 2 | —CH₂— |
| 455 | —CH₂CH(CH₃) | —H | —H | —CH₃ | 1 | 2 | —CH₂— |
| 456 | —C(═O)CH₃ | —H | —H | —CH₃ | 1 | 2 | —CH₂— |
| 457 | —C(═O)CF₃ | —H | —H | —CH₃ | 1 | 2 | —CH₂— |
| 458 | —C(═O)OCH₃ | —H | —H | —CH₃ | 1 | 2 | —CH₂— |
| 459 | —C(═O)OC(CH₃)₃ | —H | —H | —CH₃ | 1 | 2 | —CH₂— |
| 460 | —S(═O)₂CH₃ | —H | —H | —CH₃ | 1 | 2 | —CH₂— |
| 461 | 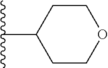 [SMILES: *C1CCOCC1] | —H | —H | —CH₃ | 1 | 2 | —CH₂— |
| 462 | 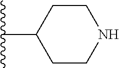 [SMILES: *C1CCNCC1] | —H | —H | —CH₃ | 1 | 2 | —CH₂— |
| 463 | 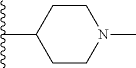 [SMILES: *C1CCN(C)CC1] | —H | —H | —CH₃ | 1 | 2 | —CH₂— |
| 464 | 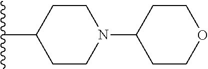 [SMILES: *C1CCN(C2CCOCC2)CC1] | —H | —H | —CH₃ | 1 | 2 | —CH₂— |
| 465 | 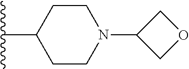 [SMILES: *C1CCN(C2COC2)CC1] | —H | —H | —CH₃ | 1 | 2 | —CH₂— |
| 466 | —H | —H | —H | —D | 1 | 2 | —CH₂— |
| 467 | —CH₃ | —H | —H | —D | 1 | 2 | —CH₂— |
| 468 | —CH₂CH₃ | —H | —H | —D | 1 | 2 | —CH₂— |
| 469 | —CH(CH₃) | —H | —H | —D | 1 | 2 | —CH₂— |
| 470 | —CH₂CH(CH₃) | —H | —H | —D | 1 | 2 | —CH₂— |
| 471 | —C(═O)CH₃ | —H | —H | —D | 1 | 2 | —CH₂— |
| 472 | —C(═O)CF₃ | —H | —H | —D | 1 | 2 | —CH₂— |
| 473 | —C(═O)OCH₃ | —H | —H | —D | 1 | 2 | —CH₂— |
| 474 | —C(═O)OC(CH₃)₃ | —H | —H | —D | 1 | 2 | —CH₂— |
| 475 | —S(═O)₂CH₃ | —H | —H | —D | 1 | 2 | —CH₂— |
| 476 | 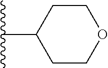 [SMILES: *C1CCOCC1] | —H | —H | —D | 1 | 2 | —CH₂— |
| 477 | 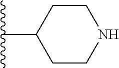 [SMILES: *C1CCNCC1] | —H | —H | —D | 1 | 2 | —CH₂— |
| 478 | 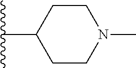 [SMILES: *C1CCN(C)CC1] | —H | —H | —D | 1 | 2 | —CH₂— |
| 479 | 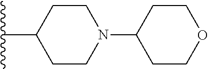 [SMILES: *C1CCN(C2CCOCC2)CC1] | —H | —H | —D | 1 | 2 | —CH₂— |
| 480 | 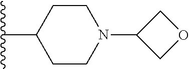 [SMILES: *C1CCN(C2COC2)CC1] | —H | —H | —D | 1 | 2 | —CH₂— |
| 481 | —H | —H | —H | —H | 1 | 1 | —C(═O)— |
| 482 | —CH₃ | —H | —H | —H | 1 | 1 | —C(═O)— |
| 483 | —CH₂CH₃ | —H | —H | —H | 1 | 1 | —C(═O)— |
| 484 | —CH(CH₃) | —H | —H | —H | 1 | 1 | —C(═O)— |
| 485 | —CH₂CH(CH₃) | —H | —H | —H | 1 | 1 | —C(═O)— |
| 486 | —C(═O)CH₃ | —H | —H | —H | 1 | 1 | —C(═O)— |
| 487 | —C(═O)CF₃ | —H | —H | —H | 1 | 1 | —C(═O)— |
| 488 | —C(═O)OCH₃ | —H | —H | —H | 1 | 1 | —C(═O)— |
| 489 | —C(═O)OC(CH₃)₃ | —H | —H | —H | 1 | 1 | —C(═O)— |
| 490 | —S(═O)₂CH₃ | —H | —H | —H | 1 | 1 | —C(═O)— |
| 491 | 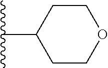 [SMILES: *C1CCOCC1] | —H | —H | —H | 1 | 1 | —C(═O)— |
| 492 | 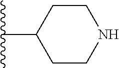 [SMILES: *C1CCNCC1] | —H | —H | —H | 1 | 1 | —C(═O)— |
| 493 | 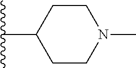 [SMILES: *C1CCN(C)CC1] | —H | —H | —H | 1 | 1 | —C(═O)— |
| 494 | 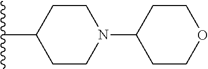 [SMILES: *C1CCN(C2CCOCC2)CC1] | —H | —H | —H | 1 | 1 | —C(═O)— |
| 495 | 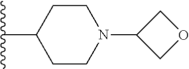 [SMILES: *C1CCN(C2COC2)CC1] | —H | —H | —H | 1 | 1 | —C(═O)— |
| 496 | —H | —H | —H | —F | 1 | 1 | —C(═O)— |
| 497 | —CH₃ | —H | —H | —F | 1 | 1 | —C(═O)— |
| 498 | —CH₂CH₃ | —H | —H | —F | 1 | 1 | —C(═O)— |
| 499 | —CH(CH₃) | —H | —H | —F | 1 | 1 | —C(═O)— |
| 500 | —CH₂CH(CH₃) | —H | —H | —F | 1 | 1 | —C(═O)— |
| 501 | —C(═O)CH₃ | —H | —H | —F | 1 | 1 | —C(═O)— |
| 502 | —C(═O)CF₃ | —H | —H | —F | 1 | 1 | —C(═O)— |
| 503 | —C(═O)OCH₃ | —H | —H | —F | 1 | 1 | —C(═O)— |
| 504 | —C(═O)OC(CH₃)₃ | —H | —H | —F | 1 | 1 | —C(═O)— |
| 505 | —S(═O)₂CH₃ | —H | —H | —F | 1 | 1 | —C(═O)— |
| 506 | 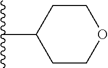 [SMILES: *C1CCOCC1] | —H | —H | —F | 1 | 1 | —C(═O)— |
| 507 | 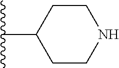 [SMILES: *C1CCNCC1] | —H | —H | —F | 1 | 1 | —C(═O)— |
| 508 | 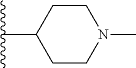 [SMILES: *C1CCN(C)CC1] | —H | —H | —F | 1 | 1 | —C(═O)— |
| 509 | 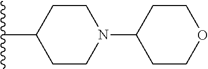 [SMILES: *C1CCN(C2CCOCC2)CC1] | —H | —H | —F | 1 | 1 | —C(═O)— |
| 510 | 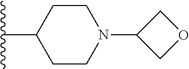 [SMILES: *C1CCN(C2COC2)CC1] | —H | —H | —F | 1 | 1 | —C(═O)— |
| 511 | —H | —H | —H | —CH₃ | 1 | 1 | —C(═O)— |
| 512 | —CH₃ | —H | —H | —CH₃ | 1 | 1 | —C(═O)— |
| 513 | —CH₂CH₃ | —H | —H | —CH₃ | 1 | 1 | —C(═O)— |
| 514 | —CH(CH₃) | —H | —H | —CH₃ | 1 | 1 | —C(═O)— |
| 515 | —CH₂CH(CH₃) | —H | —H | —CH₃ | 1 | 1 | —C(═O)— |
| 516 | —C(═O)CH₃ | —H | —H | —CH₃ | 1 | 1 | —C(═O)— |
| 517 | —C(═O)CF₃ | —H | —H | —CH₃ | 1 | 1 | —C(═O)— |
| 518 | —C(═O)OCH₃ | —H | —H | —CH₃ | 1 | 1 | —C(═O)— |
| 519 | —C(═O)OC(CH₃)₃ | —H | —H | —CH₃ | 1 | 1 | —C(═O)— |
| 520 | —S(═O)₂CH₃ | —H | —H | —CH₃ | 1 | 1 | —C(═O)— |
| 521 | 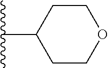 [SMILES: *C1CCOCC1] | —H | —H | —CH₃ | 1 | 1 | —C(═O)— |
| 522 | 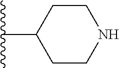 [SMILES: *C1CCNCC1] | —H | —H | —CH₃ | 1 | 1 | —C(═O)— |
| 523 | 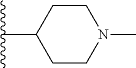 [SMILES: *C1CCN(C)CC1] | —H | —H | —CH₃ | 1 | 1 | —C(═O)— |
| 524 | 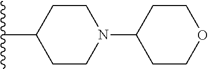 [SMILES: *C1CCN(C2CCOCC2)CC1] | —H | —H | —CH₃ | 1 | 1 | —C(═O)— |
| 525 | 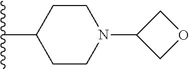 [SMILES: *C1CCN(C2COC2)CC1] | —H | —H | —CH₃ | 1 | 1 | —C(═O)— |
| 526 | —H | —H | —H | —D | 1 | 1 | —C(═O)— |
| 527 | —CH₃ | —H | —H | —D | 1 | 1 | —C(═O)— |
| 528 | —CH₂CH₃ | —H | —H | —D | 1 | 1 | —C(═O)— |
| 529 | —CH(CH₃) | —H | —H | —D | 1 | 1 | —C(═O)— |
| 530 | —CH₂CH(CH₃) | —H | —H | —D | 1 | 1 | —C(═O)— |
| 531 | —C(═O)CH₃ | —H | —H | —D | 1 | 1 | —C(═O)— |
| 532 | —C(═O)CF₃ | —H | —H | —D | 1 | 1 | —C(═O)— |
| 533 | —C(═O)OCH₃ | —H | —H | —D | 1 | 1 | —C(═O)— |
| 534 | —C(═O)OC(CH₃)₃ | —H | —H | —D | 1 | 1 | —C(═O)— |
| 535 | —S(═O)₂CH₃ | —H | —H | —D | 1 | 1 | —C(═O)— |
| 536 | 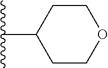 [SMILES: *C1CCOCC1] | —H | —H | —D | 1 | 1 | —C(═O)— |
| 537 | 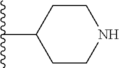 [SMILES: *C1CCNCC1] | —H | —H | —D | 1 | 1 | —C(═O)— |
| 538 | 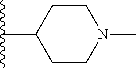 [SMILES: *C1CCN(C)CC1] | —H | —H | —D | 1 | 1 | —C(═O)— |
| 539 | 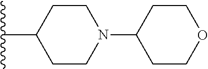 [SMILES: *C1CCN(C2CCOCC2)CC1] | —H | —H | —D | 1 | 1 | —C(═O)— |
| 540 | 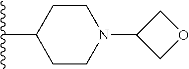 [SMILES: *C1CCN(C2COC2)CC1] | —H | —H | —D | 1 | 1 | —C(═O)— |
| 541 | —H | —H | —H | —H | 2 | 1 | —C(═O)— |
| 542 | —CH₃ | —H | —H | —H | 2 | 1 | —C(═O)— |
| 543 | —CH₂CH₃ | —H | —H | —H | 2 | 1 | —C(═O)— |
| 544 | —CH(CH₃) | —H | —H | —H | 2 | 1 | —C(═O)— |
| 545 | —CH₂CH(CH₃) | —H | —H | —H | 2 | 1 | —C(═O)— |
| 546 | —C(═O)CH₃ | —H | —H | —H | 2 | 1 | —C(═O)— |
| 547 | —C(═O)CF₃ | —H | —H | —H | 2 | 1 | —C(═O)— |
| 548 | —C(═O)OCH₃ | —H | —H | —H | 2 | 1 | —C(═O)— |
| 549 | —C(═O)OC(CH₃)₃ | —H | —H | —H | 2 | 1 | —C(═O)— |
| 550 | —S(═O)₂CH₃ | —H | —H | —H | 2 | 1 | —C(═O)— |
| 551 | 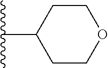 [SMILES: *C1CCOCC1] | —H | —H | —H | 2 | 1 | —C(═O)— |
| 552 | 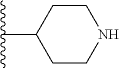 [SMILES: *C1CCNCC1] | —H | —H | —H | 2 | 1 | —C(═O)— |
| 553 | 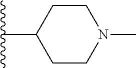 [SMILES: *C1CCN(C)CC1] | —H | —H | —H | 2 | 1 | —C(═O)— |
| 554 | 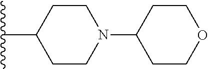 [SMILES: *C1CCN(C2CCOCC2)CC1] | —H | —H | —H | 2 | 1 | —C(═O)— |
| 555 | 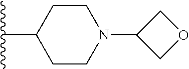 [SMILES: *C1CCN(C2COC2)CC1] | —H | —H | —H | 2 | 1 | —C(═O)— |
| 556 | —H | —H | —H | —F | 2 | 1 | —C(═O)— |
| 557 | —CH₃ | —H | —H | —F | 2 | 1 | —C(═O)— |
| 558 | —CH₂CH₃ | —H | —H | —F | 2 | 1 | —C(═O)— |
| 559 | —CH(CH₃) | —H | —H | —F | 2 | 1 | —C(═O)— |
| 560 | —CH₂CH(CH₃) | —H | —H | —F | 2 | 1 | —C(═O)— |
| 561 | —C(═O)CH₃ | —H | —H | —F | 2 | 1 | —C(═O)— |
| 562 | —C(═O)CF₃ | —H | —H | —F | 2 | 1 | —C(═O)— |
| 563 | —C(═O)OCH₃ | —H | —H | —F | 2 | 1 | —C(═O)— |
| 564 | —C(═O)OC(CH₃)₃ | —H | —H | —F | 2 | 1 | —C(═O)— |
| 565 | —S(═O)₂CH₃ | —H | —H | —F | 2 | 1 | —C(═O)— |
| 566 | 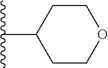 [SMILES: *C1CCOCC1] | —H | —H | —F | 2 | 1 | —C(═O)— |
| 567 | 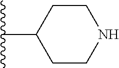 [SMILES: *C1CCNCC1] | —H | —H | —F | 2 | 1 | —C(═O)— |
| 568 | 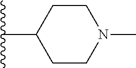 [SMILES: *C1CCN(C)CC1] | —H | —H | —F | 2 | 1 | —C(═O)— |
| 569 | 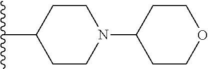 [SMILES: *C1CCN(C2CCOCC2)CC1] | —H | —H | —F | 2 | 1 | —C(═O)— |
| 570 | 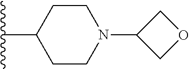 [SMILES: *C1CCN(C2COC2)CC1] | —H | —H | —F | 2 | 1 | —C(═O)— |
| 571 | —H | —H | —H | —CH₃ | 2 | 1 | —C(═O)— |
| 572 | —CH₃ | —H | —H | —CH₃ | 2 | 1 | —C(═O)— |
| 573 | —CH₂CH₃ | —H | —H | —CH₃ | 2 | 1 | —C(═O)— |
| 574 | —CH(CH₃) | —H | —H | —CH₃ | 2 | 1 | —C(═O)— |
| 575 | —CH₂CH(CH₃) | —H | —H | —CH₃ | 2 | 1 | —C(═O)— |
| 576 | —C(═O)CH₃ | —H | —H | —CH₃ | 2 | 1 | —C(═O)— |
| 577 | —C(═O)CF₃ | —H | —H | —CH₃ | 2 | 1 | —C(═O)— |
| 578 | —C(═O)OCH₃ | —H | —H | —CH₃ | 2 | 1 | —C(═O)— |
| 579 | —C(═O)OC(CH₃)₃ | —H | —H | —CH₃ | 2 | 1 | —C(═O)— |
| 580 | —S(═O)₂CH₃ | —H | —H | —CH₃ | 2 | 1 | —C(═O)— |
| 581 | 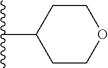 [SMILES: *C1CCOCC1] | —H | —H | —CH₃ | 2 | 1 | —C(═O)— |
| 582 | 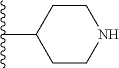 [SMILES: *C1CCNCC1] | —H | —H | —CH₃ | 2 | 1 | —C(═O)— |
| 583 | 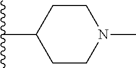 [SMILES: *C1CCN(C)CC1] | —H | —H | —CH₃ | 2 | 1 | —C(═O)— |
| 584 | 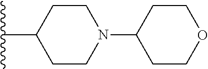 [SMILES: *C1CCN(C2CCOCC2)CC1] | —H | —H | —CH₃ | 2 | 1 | —C(═O)— |
| 585 | 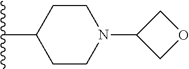 [SMILES: *C1CCN(C2COC2)CC1] | —H | —H | —CH₃ | 2 | 1 | —C(═O)— |
| 586 | —H | —H | —H | —D | 2 | 1 | —C(═O)— |
| 587 | —CH₃ | —H | —H | —D | 2 | 1 | —C(═O)— |
| 588 | —CH₂CH₃ | —H | —H | —D | 2 | 1 | —C(═O)— |
| 589 | —CH(CH₃) | —H | —H | —D | 2 | 1 | —C(═O)— |
| 590 | —CH₂CH(CH₃) | —H | —H | —D | 2 | 1 | —C(═O)— |
| 591 | —C(═O)CH₃ | —H | —H | —D | 2 | 1 | —C(═O)— |
| 592 | —C(═O)CF₃ | —H | —H | —D | 2 | 1 | —C(═O)— |
| 593 | —C(═O)OCH₃ | —H | —H | —D | 2 | 1 | —C(═O)— |
| 594 | —C(═O)OC(CH₃)₃ | —H | —H | —D | 2 | 1 | —C(═O)— |
| 595 | —S(═O)₂CH₃ | —H | —H | —D | 2 | 1 | —C(═O)— |
| 596 | 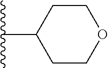 [SMILES: *C1CCOCC1] | —H | —H | —D | 2 | 1 | —C(═O)— |
| 597 | 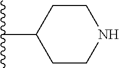 [SMILES: *C1CCNCC1] | —H | —H | —D | 2 | 1 | —C(═O)— |
| 598 | 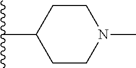 [SMILES: *C1CCN(C)CC1] | —H | —H | —D | 2 | 1 | —C(═O)— |
| 599 | 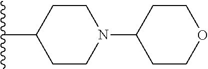 [SMILES: *C1CCN(C2CCOCC2)CC1] | —H | —H | —D | 2 | 1 | —C(═O)— |
| 600 | 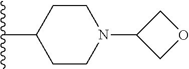 [SMILES: *C1CCN(C2COC2)CC1] | —H | —H | —D | 2 | 1 | —C(═O)— |
| 601 | —H | —H | —H | —H | 1 | 2 | —C(═O)— |
| 602 | —CH₃ | —H | —H | —H | 1 | 2 | —C(═O)— |
| 603 | —CH₂CH₃ | —H | —H | —H | 1 | 2 | —C(═O)— |
| 604 | —CH(CH₃) | —H | —H | —H | 1 | 2 | —C(═O)— |
| 605 | —CH₂CH(CH₃) | —H | —H | —H | 1 | 2 | —C(═O)— |
| 606 | —C(═O)CH₃ | —H | —H | —H | 1 | 2 | —C(═O)— |
| 607 | —C(═O)CF₃ | —H | —H | —H | 1 | 2 | —C(═O)— |
| 608 | —C(═O)OCH₃ | —H | —H | —H | 1 | 2 | —C(═O)— |
| 609 | —C(═O)OC(CH₃)₃ | —H | —H | —H | 1 | 2 | —C(═O)— |
| 610 | —S(═O)₂CH₃ | —H | —H | —H | 1 | 2 | —C(═O)— |
| 611 | 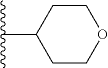 [SMILES: *C1CCOCC1] | —H | —H | —H | 1 | 2 | —C(═O)— |
| 612 | 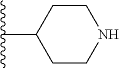 [SMILES: *C1CCNCC1] | —H | —H | —H | 1 | 2 | —C(═O)— |
| 613 | 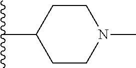 [SMILES: *C1CCN(C)CC1] | —H | —H | —H | 1 | 2 | —C(═O)— |
| 614 | 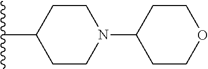 [SMILES: *C1CCN(C2CCOCC2)CC1] | —H | —H | —H | 1 | 2 | —C(═O)— |
| 615 | 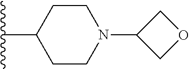 [SMILES: *C1CCN(C2COC2)CC1] | —H | —H | —H | 1 | 2 | —C(═O)— |
| 616 | —H | —H | —H | —F | 1 | 2 | —C(═O)— |
| 617 | —CH₃ | —H | —H | —F | 1 | 2 | —C(═O)— |
| 618 | —CH₂CH₃ | —H | —H | —F | 1 | 2 | —C(═O)— |
| 619 | —CH(CH₃) | —H | —H | —F | 1 | 2 | —C(═O)— |
| 620 | —CH₂CH(CH₃) | —H | —H | —F | 1 | 2 | —C(═O)— |
| 621 | —C(═O)CH₃ | —H | —H | —F | 1 | 2 | —C(═O)— |
| 622 | —C(═O)CF₃ | —H | —H | —F | 1 | 2 | —C(═O)— |
| 623 | —C(═O)OCH₃ | —H | —H | —F | 1 | 2 | —C(═O)— |
| 624 | —C(═O)OC(CH₃)₃ | —H | —H | —F | 1 | 2 | —C(═O)— |
| 625 | —S(═O)₂CH₃ | —H | —H | —F | 1 | 2 | —C(═O)— |
| 626 | 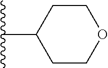 [SMILES: *C1CCOCC1] | —H | —H | —F | 1 | 2 | —C(═O)— |
| 627 | 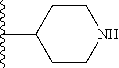 [SMILES: *C1CCNCC1] | —H | —H | —F | 1 | 2 | —C(═O)— |
| 628 | 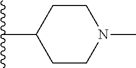 [SMILES: *C1CCN(C)CC1] | —H | —H | —F | 1 | 2 | —C(═O)— |
| 629 | 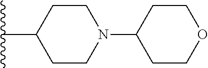 [SMILES: *C1CCN(C2CCOCC2)CC1] | —H | —H | —F | 1 | 2 | —C(═O)— |
| 630 | 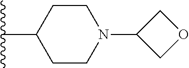 [SMILES: *C1CCN(C2COC2)CC1] | —H | —H | —F | 1 | 2 | —C(═O)— |
| 631 | —H | —H | —H | —CH₃ | 1 | 2 | —C(═O)— |
| 632 | —CH₃ | —H | —H | —CH₃ | 1 | 2 | —C(═O)— |
| 633 | —CH₂CH₃ | —H | —H | —CH₃ | 1 | 2 | —C(═O)— |
| 634 | —CH(CH₃) | —H | —H | —CH₃ | 1 | 2 | —C(═O)— |
| 635 | —CH₂CH(CH₃) | —H | —H | —CH₃ | 1 | 2 | —C(═O)— |
| 636 | —C(═O)CH₃ | —H | —H | —CH₃ | 1 | 2 | —C(═O)— |
| 637 | —C(═O)CF₃ | —H | —H | —CH₃ | 1 | 2 | —C(═O)— |
| 638 | —C(═O)OCH₃ | —H | —H | —CH₃ | 1 | 2 | —C(═O)— |
| 639 | —C(═O)OC(CH₃)₃ | —H | —H | —CH₃ | 1 | 2 | —C(═O)— |
| 640 | —S(═O)₂CH₃ | —H | —H | —CH₃ | 1 | 2 | —C(═O)— |
| 641 | 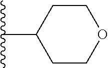 [SMILES: *C1CCOCC1] | —H | —H | —CH₃ | 1 | 2 | —C(═O)— |
| 642 | 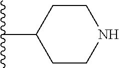 [SMILES: *C1CCNCC1] | —H | —H | —CH₃ | 1 | 2 | —C(═O)— |
| 643 | 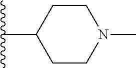 [SMILES: *C1CCN(C)CC1] | —H | —H | —CH₃ | 1 | 2 | —C(═O)— |
| 644 | 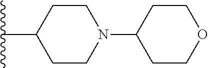 [SMILES: *C1CCN(C2CCOCC2)CC1] | —H | —H | —CH₃ | 1 | 2 | —C(═O)— |
| 645 | 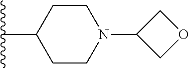 [SMILES: *C1CCN(C2COC2)CC1] | —H | —H | —CH₃ | 1 | 2 | —C(═O)— |
| 631 | —H | —H | —H | —D | 1 | 2 | —C(═O)— |
| 632 | —CH₃ | —H | —H | —D | 1 | 2 | —C(═O)— |
| 633 | —CH₂CH₃ | —H | —H | —D | 1 | 2 | —C(═O)— |
| 634 | —CH(CH₃) | —H | —H | —D | 1 | 2 | —C(═O)— |
| 635 | —CH₂CH(CH₃) | —H | —H | —D | 1 | 2 | —C(═O)— |
| 636 | —C(═O)CH₃ | —H | —H | —D | 1 | 2 | —C(═O)— |
| 637 | —C(═O)CF₃ | —H | —H | —D | 1 | 2 | —C(═O)— |
| 638 | —C(═O)OCH₃ | —H | —H | —D | 1 | 2 | —C(═O)— |
| 639 | —C(═O)OC(CH₃)₃ | —H | —H | —D | 1 | 2 | —C(═O)— |
| 640 | —S(═O)₂CH₃ | —H | —H | —D | 1 | 2 | —C(═O)— |
| 641 | 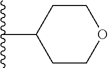 [SMILES: *C1CCOCC1] | —H | —H | —D | 1 | 2 | —C(═O)— |
| 642 | 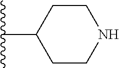 [SMILES: *C1CCNCC1] | —H | —H | —D | 1 | 2 | —C(═O)— |
| 643 | 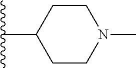 [SMILES: *C1CCN(C)CC1] | —H | —H | —D | 1 | 2 | —C(═O)— |
| 644 | 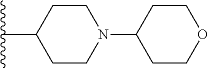 [SMILES: *C1CCN(C2CCOCC2)CC1] | —H | —H | —D | 1 | 2 | —C(═O)— |
| 645 | 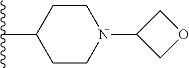 [SMILES: *C1CCN(C2COC2)CC1] | —H | —H | —D | 1 | 2 | —C(═O)— |
| 858 | —H | —H | —H | —H | 3 | 1 | —C(═O)— |
| 859 | —H | —H | —H | —H | 2 | 2 | —C(═O)— |

In another embodiment, Compounds of the Disclosure are compounds of Formula IV, wherein R¹, R^{2a}, R^{2b}, R³, m, n, and Z are as defined in Table 3, or a pharmaceutically acceptable salt or solvate thereof.

TABLE 3
IV

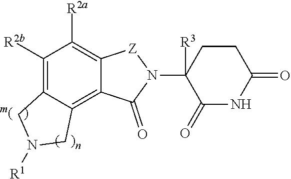

| Cpd. No. | R¹ | R^{2a} | R^{2d} | R³ | m | n | Z |
|---|---|---|---|---|---|---|---|
| 646 | —H | —H | —H | —H | 1 | 1 | —CH₂— |
| 647 | —CH₃ | —H | —H | —H | 1 | 1 | —CH₂— |
| 648 | —CH₂CH₃ | —H | —H | —H | 1 | 1 | —CH₂— |
| 649 | —CH(CH₃) | —H | —H | —H | 1 | 1 | —CH₂— |
| 650 | —CH₂CH(CH₃) | —H | —H | —H | 1 | 1 | —CH₂— |
| 651 | —C(═O)CH₃ | —H | —H | —H | 1 | 1 | —CH₂— |
| 652 | —C(═O)CF₃ | —H | —H | —H | 1 | 1 | —CH₂— |
| 653 | —C(═O)OCH₃ | —H | —H | —H | 1 | 1 | —CH₂— |
| 654 | —C(═O)OC(CH₃)₃ | —H | —H | —H | 1 | 1 | —CH₂— |
| 655 | —S(═O)₂CH₃ | —H | —H | —H | 1 | 1 | —CH₂— |
| 656 | 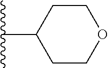 [SMILES: *C1CCOCC1] | —H | —H | —H | 1 | 1 | —CH₂— |
| 657 | 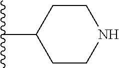 [SMILES: *C1CCNCC1] | —H | —H | —H | 1 | 1 | —CH₂— |
| 658 | 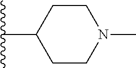 [SMILES: *C1CCN(C)CC1] | —H | —H | —H | 1 | 1 | —CH₂— |
| 659 | 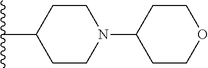 [SMILES: *C1CCN(C2CCOCC2)CC1] | —H | —H | —H | 1 | 1 | —CH₂— |
| 660 | 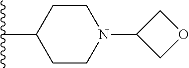 [SMILES: *C1CCN(C2COC2)CC1] | —H | —H | —H | 1 | 1 | —CH₂— |
| 661 | —H | —H | —H | —F | 1 | 1 | —CH₂— |
| 662 | —CH₃ | —H | —H | —F | 1 | 1 | —CH₂— |
| 663 | —CH₂CH₃ | —H | —H | —F | 1 | 1 | —CH₂— |
| 664 | —CH(CH₃) | —H | —H | —F | 1 | 1 | —CH₂— |
| 665 | —CH₂CH(CH₃) | —H | —H | —F | 1 | 1 | —CH₂— |
| 666 | —C(═O)CH₃ | —H | —H | —F | 1 | 1 | —CH₂— |
| 667 | —C(═O)CF₃ | —H | —H | —F | 1 | 1 | —CH₂— |
| 668 | —C(═O)OCH₃ | —H | —H | —F | 1 | 1 | —CH₂— |
| 669 | —C(═O)OC(CH₃)₃ | —H | —H | —F | 1 | 1 | —CH₂— |
| 670 | —S(═O)₂CH₃ | —H | —H | —F | 1 | 1 | —CH₂— |
| 671 | 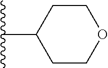 [SMILES: *C1CCOCC1] | —H | —H | —F | 1 | 1 | —CH₂— |
| 672 | 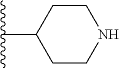 [SMILES: *C1CCNCC1] | —H | —H | —F | 1 | 1 | —CH₂— |
| 673 | 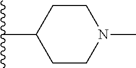 [SMILES: *C1CCN(C)CC1] | —H | —H | —F | 1 | 1 | —CH₂— |
| 674 | 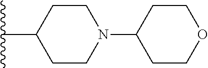 [SMILES: *C1CCN(C2CCOCC2)CC1] | —H | —H | —F | 1 | 1 | —CH₂— |
| 675 | 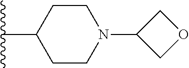 [SMILES: *C1CCN(C2COC2)CC1] | —H | —H | —F | 1 | 1 | —CH₂— |
| 676 | —H | —H | —H | —CH₃ | 1 | 1 | —CH₂— |
| 677 | —CH₃ | —H | —H | —CH₃ | 1 | 1 | —CH₂— |
| 678 | —CH₂CH₃ | —H | —H | —CH₃ | 1 | 1 | —CH₂— |
| 679 | —CH(CH₃) | —H | —H | —CH₃ | 1 | 1 | —CH₂— |
| 680 | —CH₂CH(CH₃) | —H | —H | —CH₃ | 1 | 1 | —CH₂— |
| 681 | —C(═O)CH₃ | —H | —H | —CH₃ | 1 | 1 | —CH₂— |
| 682 | —C(═O)CF₃ | —H | —H | —CH₃ | 1 | 1 | —CH₂— |
| 683 | —C(═O)OCH₃ | —H | —H | —CH₃ | 1 | 1 | —CH₂— |
| 684 | —C(═O)OC(CH₃)₃ | —H | —H | —CH₃ | 1 | 1 | —CH₂— |
| 685 | —S(═O)₂CH₃ | —H | —H | —CH₃ | 1 | 1 | —CH₂— |
| 686 | 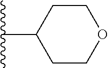 [SMILES: *C1CCOCC1] | —H | —H | —CH₃ | 1 | 1 | —CH₂— |
| 687 | 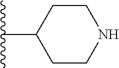 [SMILES: *C1CCNCC1] | —H | —H | —CH₃ | 1 | 1 | —CH₂— |
| 688 | 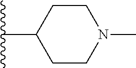 [SMILES: *C1CCN(C)CC1] | —H | —H | —CH₃ | 1 | 1 | —CH₂— |
| 689 | 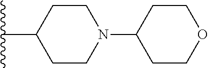 [SMILES: *C1CCN(C2CCOCC2)CC1] | —H | —H | —CH₃ | 1 | 1 | —CH₂— |
| 690 | 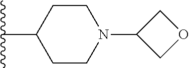 [SMILES: *C1CCN(C2COC2)CC1] | —H | —H | —CH₃ | 1 | 1 | —CH₂— |
| 691 | —H | —H | —H | —D | 1 | 1 | —CH₂— |
| 692 | —CH₃ | —H | —H | —D | 1 | 1 | —CH₂— |
| 693 | —CH₂CH₃ | —H | —H | —D | 1 | 1 | —CH₂— |
| 694 | —CH(CH₃) | —H | —H | —D | 1 | 1 | —CH₂— |
| 695 | —CH₂CH(CH₃) | —H | —H | —D | 1 | 1 | —CH₂— |
| 696 | —C(═O)CH₃ | —H | —H | —D | 1 | 1 | —CH₂— |
| 697 | —C(═O)CF₃ | —H | —H | —D | 1 | 1 | —CH₂— |
| 698 | —C(═O)OCH₃ | —H | —H | —D | 1 | 1 | —CH₂— |
| 699 | —C(═O)OC(CH₃)₃ | —H | —H | —D | 1 | 1 | —CH₂— |
| 700 | —S(═O)₂CH₃ | —H | —H | —D | 1 | 1 | —CH₂— |
| 701 | 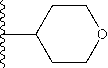 [SMILES: *C1CCOCC1] | —H | —H | —D | 1 | 1 | —CH₂— |
| 702 | 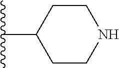 [SMILES: *C1CCNCC1] | —H | —H | —D | 1 | 1 | —CH₂— |
| 703 | 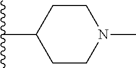 [SMILES: *C1CCN(C)CC1] | —H | —H | —D | 1 | 1 | —CH₂— |
| 704 | 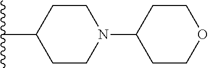 [SMILES: *C1CCN(C2CCOCC2)CC1] | —H | —H | —D | 1 | 1 | —CH₂— |
| 705 | 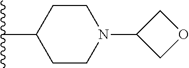 [SMILES: *C1CCN(C2COC2)CC1] | —H | —H | —D | 1 | 1 | —CH₂— |
| 706 | —H | —H | —H | —H | 2 | 1 | —CH₂— |
| 707 | —CH₃ | —H | —H | —H | 2 | 1 | —CH₂— |
| 708 | —CH₂CH₃ | —H | —H | —H | 2 | 1 | —CH₂— |
| 709 | —CH(CH₃) | —H | —H | —H | 2 | 1 | —CH₂— |
| 710 | —CH₂CH(CH₃) | —H | —H | —H | 2 | 1 | —CH₂— |
| 711 | —C(═O)CH₃ | —H | —H | —H | 2 | 1 | —CH₂— |
| 712 | —C(═O)CF₃ | —H | —H | —H | 2 | 1 | —CH₂— |
| 713 | —C(═O)OCH₃ | —H | —H | —H | 2 | 1 | —CH₂— |
| 714 | —C(═O)OC(CH₃)₃ | —H | —H | —H | 2 | 1 | —CH₂— |
| 715 | —S(═O)₂CH₃ | —H | —H | —H | 2 | 1 | —CH₂— |
| 716 | 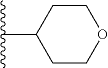 [SMILES: *C1CCOCC1] | —H | —H | —H | 2 | 1 | —CH₂— |
| 717 | 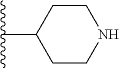 [SMILES: *C1CCNCC1] | —H | —H | —H | 2 | 1 | —CH₂— |
| 718 | 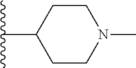 [SMILES: *C1CCN(C)CC1] | —H | —H | —H | 2 | 1 | —CH₂— |
| 719 | 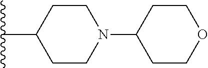 [SMILES: *C1CCN(C2CCOCC2)CC1] | —H | —H | —H | 2 | 1 | —CH₂— |
| 720 | 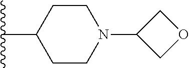 [SMILES: *C1CCN(C2COC2)CC1] | —H | —H | —H | 2 | 1 | —CH₂— |
| 721 | —H | —H | —H | —F | 2 | 1 | —CH₂— |
| 722 | —CH₃ | —H | —H | —F | 2 | 1 | —CH₂— |
| 723 | —CH₂CH₃ | —H | —H | —F | 2 | 1 | —CH₂— |
| 724 | —CH(CH₃) | —H | —H | —F | 2 | 1 | —CH₂— |
| 725 | —CH₂CH(CH₃) | —H | —H | —F | 2 | 1 | —CH₂— |
| 726 | —C(═O)CH₃ | —H | —H | —F | 2 | 1 | —CH₂— |
| 727 | —C(═O)CF₃ | —H | —H | —F | 2 | 1 | —CH₂— |
| 728 | —C(═O)OCH₃ | —H | —H | —F | 2 | 1 | —CH₂— |
| 729 | —C(═O)OC(CH₃)₃ | —H | —H | —F | 2 | 1 | —CH₂— |
| 730 | —S(═O)₂CH₃ | —H | —H | —F | 2 | 1 | —CH₂— |
| 731 | 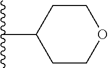 [SMILES: *C1CCOCC1] | —H | —H | —F | 2 | 1 | —CH₂— |
| 732 | 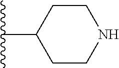 [SMILES: *C1CCNCC1] | —H | —H | —F | 2 | 1 | —CH₂— |
| 733 | 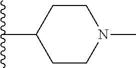 [SMILES: *C1CCN(C)CC1] | —H | —H | —F | 2 | 1 | —CH₂— |
| 734 | 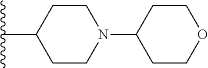 [SMILES: *C1CCN(C2CCOCC2)CC1] | —H | —H | —F | 2 | 1 | —CH₂— |
| 735 | 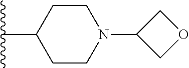 [SMILES: *C1CCN(C2COC2)CC1] | —H | —H | —F | 2 | 1 | —CH₂— |
| 736 | —H | —H | —H | —CH₃ | 2 | 1 | —CH₂— |
| 737 | —CH₃ | —H | —H | —CH₃ | 2 | 1 | —CH₂— |
| 738 | —CH₂CH₃ | —H | —H | —CH₃ | 2 | 1 | —CH₂— |
| 739 | —CH(CH₃) | —H | —H | —CH₃ | 2 | 1 | —CH₂— |
| 740 | —CH₂CH(CH₃) | —H | —H | —CH₃ | 2 | 1 | —CH₂— |
| 741 | —C(═O)CH₃ | —H | —H | —CH₃ | 2 | 1 | —CH₂— |
| 742 | —C(═O)CF₃ | —H | —H | —CH₃ | 2 | 1 | —CH₂— |
| 743 | —C(═O)OCH₃ | —H | —H | —CH₃ | 2 | 1 | —CH₂— |
| 744 | —C(═O)OC(CH₃)₃ | —H | —H | —CH₃ | 2 | 1 | —CH₂— |
| 745 | —S(═O)₂CH₃ | —H | —H | —CH₃ | 2 | 1 | —CH₂— |
| 746 | 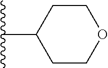 [SMILES: *C1CCOCC1] | —H | —H | —CH₃ | 2 | 1 | —CH₂— |
| 747 | 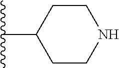 [SMILES: *C1CCNCC1] | —H | —H | —CH₃ | 2 | 1 | —CH₂— |
| 748 | 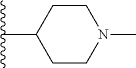 [SMILES: *C1CCN(C)CC1] | —H | —H | —CH₃ | 2 | 1 | —CH₂— |
| 749 | 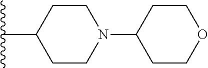 [SMILES: *C1CCN(C2CCOCC2)CC1] | —H | —H | —CH₃ | 2 | 1 | —CH₂— |
| 750 | 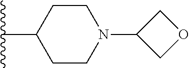 [SMILES: *C1CCN(C2COC2)CC1] | —H | —H | —CH₃ | 2 | 1 | —CH₂— |
| 751 | —H | —H | —H | —D | 2 | 1 | —CH₂— |
| 752 | —CH₃ | —H | —H | —D | 2 | 1 | —CH₂— |
| 753 | —CH₂CH₃ | —H | —H | —D | 2 | 1 | —CH₂— |
| 754 | —CH(CH₃) | —H | —H | —D | 2 | 1 | —CH₂— |
| 755 | —CH₂CH(CH₃) | —H | —H | —D | 2 | 1 | —CH₂— |
| 756 | —C(═O)CH₃ | —H | —H | —D | 2 | 1 | —CH₂— |
| 757 | —C(═O)CF₃ | —H | —H | —D | 2 | 1 | —CH₂— |
| 758 | —C(═O)OCH₃ | —H | —H | —D | 2 | 1 | —CH₂— |
| 759 | —C(═O)OC(CH₃)₃ | —H | —H | —D | 2 | 1 | —CH₂— |
| 760 | —S(═O)₂CH₃ | —H | —H | —D | 2 | 1 | —CH₂— |
| 761 | 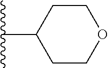 [SMILES: *C1CCOCC1] | —H | —H | —D | 2 | 1 | —CH₂— |
| 762 | 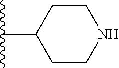 [SMILES: *C1CCNCC1] | —H | —H | —D | 2 | 1 | —CH₂— |
| 763 | 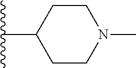 [SMILES: *C1CCN(C)CC1] | —H | —H | —D | 2 | 1 | —CH₂— |
| 764 | 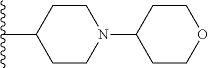 [SMILES: *C1CCN(C2CCOCC2)CC1] | —H | —H | —D | 2 | 1 | —CH₂— |
| 765 | 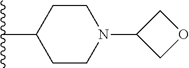 [SMILES: *C1CCN(C2COC2)CC1] | —H | —H | —D | 2 | 1 | —CH₂— |
| 766 | —H | —H | —H | —H | 1 | 2 | —CH₂— |
| 767 | —CH₃ | —H | —H | —H | 1 | 2 | —CH₂— |
| 768 | —CH₂CH₃ | —H | —H | —H | 1 | 2 | —CH₂— |
| 769 | —CH(CH₃) | —H | —H | —H | 1 | 2 | —CH₂— |
| 770 | —CH₂CH(CH₃) | —H | —H | —H | 1 | 2 | —CH₂— |
| 771 | —C(═O)CH₃ | —H | —H | —H | 1 | 2 | —CH₂— |
| 772 | —C(═O)CF₃ | —H | —H | —H | 1 | 2 | —CH₂— |
| 773 | —C(═O)OCH₃ | —H | —H | —H | 1 | 2 | —CH₂— |
| 774 | —C(═O)OC(CH₃)₃ | —H | —H | —H | 1 | 2 | —CH₂— |
| 775 | —S(═O)₂CH₃ | —H | —H | —H | 1 | 2 | —CH₂— |
| 776 | 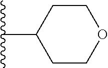 [SMILES: *C1CCOCC1] | —H | —H | —H | 1 | 2 | —CH₂— |
| 777 | 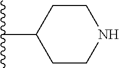 [SMILES: *C1CCNCC1] | —H | —H | —H | 1 | 2 | —CH₂— |
| 778 | 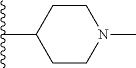 [SMILES: *C1CCN(C)CC1] | —H | —H | —H | 1 | 2 | —CH₂— |
| 779 | 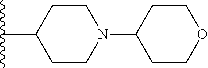 [SMILES: *C1CCN(C2CCOCC2)CC1] | —H | —H | —H | 1 | 2 | —CH₂— |
| 780 | 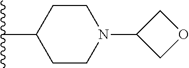 [SMILES: *C1CCN(C2COC2)CC1] | —H | —H | —H | 1 | 2 | —CH₂— |
| 781 | —H | —H | —H | —F | 1 | 2 | —CH₂— |
| 782 | —CH₃ | —H | —H | —F | 1 | 2 | —CH₂— |
| 783 | —CH₂CH₃ | —H | —H | —F | 1 | 2 | —CH₂— |
| 784 | —CH(CH₃) | —H | —H | —F | 1 | 2 | —CH₂— |
| 785 | —CH₂CH(CH₃) | —H | —H | —F | 1 | 2 | —CH₂— |
| 786 | —C(═O)CH₃ | —H | —H | —F | 1 | 2 | —CH₂— |
| 787 | —C(═O)CF₃ | —H | —H | —F | 1 | 2 | —CH₂— |
| 788 | —C(═O)OCH₃ | —H | —H | —F | 1 | 2 | —CH₂— |
| 789 | —C(═O)OC(CH₃)₃ | —H | —H | —F | 1 | 2 | —CH₂— |
| 790 | —S(═O)₂CH₃ | —H | —H | —F | 1 | 2 | —CH₂— |
| 791 | 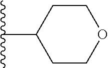 [SMILES: *C1CCOCC1] | —H | —H | —F | 1 | 2 | —CH₂— |
| 792 | 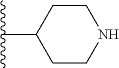 [SMILES: *C1CCNCC1] | —H | —H | —F | 1 | 2 | —CH₂— |
| 793 | 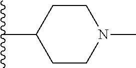 [SMILES: *C1CCN(C)CC1] | —H | —H | —F | 1 | 2 | —CH₂— |
| 794 | 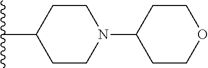 [SMILES: *C1CCN(C2CCOCC2)CC1] | —H | —H | —F | 1 | 2 | —CH₂— |
| 795 | 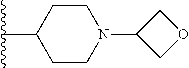 [SMILES: *C1CCN(C2COC2)CC1] | —H | —H | —F | 1 | 2 | —CH₂— |
| 796 | —H | —H | —H | —CH₃ | 1 | 2 | —CH₂— |
| 797 | —CH₃ | —H | —H | —CH₃ | 1 | 2 | —CH₂— |
| 798 | —CH₂CH₃ | —H | —H | —CH₃ | 1 | 2 | —CH₂— |
| 799 | —CH(CH₃) | —H | —H | —CH₃ | 1 | 2 | —CH₂— |
| 800 | —CH₂CH(CH₃) | —H | —H | —CH₃ | 1 | 2 | —CH₂— |
| 801 | —C(═O)CH₃ | —H | —H | —CH₃ | 1 | 2 | —CH₂— |
| 802 | —C(═O)CF₃ | —H | —H | —CH₃ | 1 | 2 | —CH₂— |
| 803 | —C(═O)OCH₃ | —H | —H | —CH₃ | 1 | 2 | —CH₂— |
| 804 | —C(═O)OC(CH₃)₃ | —H | —H | —CH₃ | 1 | 2 | —CH₂— |
| 805 | —S(═O)₂CH₃ | —H | —H | —CH₃ | 1 | 2 | —CH₂— |
| 806 | 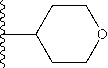 [SMILES: *C1CCOCC1] | —H | —H | —CH₃ | 1 | 2 | —CH₂— |
| 807 | 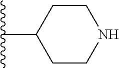 [SMILES: *C1CCNCC1] | —H | —H | —CH₃ | 1 | 2 | —CH₂— |
| 808 | 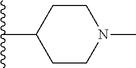 [SMILES: *C1CCN(C)CC1] | —H | —H | —CH₃ | 1 | 2 | —CH₂— |
| 809 | 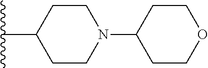 [SMILES: *C1CCN(C2CCOCC2)CC1] | —H | —H | —CH₃ | 1 | 2 | —CH₂— |
| 810 | 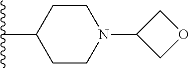 [SMILES: *C1CCN(C2COC2)CC1] | —H | —H | —CH₃ | 1 | 2 | —CH₂— |
| 811 | —H | —H | —H | —D | 1 | 2 | —CH₂— |
| 812 | —CH₃ | —H | —H | —D | 1 | 2 | —CH₂— |
| 813 | —CH₂CH₃ | —H | —H | —D | 1 | 2 | —CH₂— |
| 814 | —CH(CH₃) | —H | —H | —D | 1 | 2 | —CH₂— |
| 815 | —CH₂CH(CH₃) | —H | —H | —D | 1 | 2 | —CH₂— |
| 816 | —C(═O)CH₃ | —H | —H | —D | 1 | 2 | —CH₂— |
| 817 | —C(═O)CF₃ | —H | —H | —D | 1 | 2 | —CH₂— |
| 818 | —C(═O)OCH₃ | —H | —H | —D | 1 | 2 | —CH₂— |
| 819 | —C(═O)OC(CH₃)₃ | —H | —H | —D | 1 | 2 | —CH₂— |
| 820 | —S(═O)₂CH₃ | —H | —H | —D | 1 | 2 | —CH₂— |
| 821 | 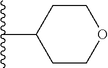 [SMILES: *C1CCOCC1] | —H | —H | —D | 1 | 2 | —CH₂— |
| 822 | 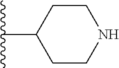 [SMILES: *C1CCNCC1] | —H | —H | —D | 1 | 2 | —CH₂— |
| 823 | 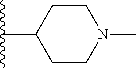 [SMILES: *C1CCN(C)CC1] | —H | —H | —D | 1 | 2 | —CH₂— |
| 824 | 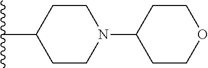 [SMILES: *C1CCN(C2CCOCC2)CC1] | —H | —H | —D | 1 | 2 | —CH₂— |
| 825 | 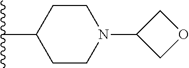 [SMILES: *C1CCN(C2COC2)CC1] | —H | —H | —D | 1 | 2 | —CH₂— |

In another embodiment, Compounds of the Disclosure are compounds of Formula IX, wherein R¹, R^{2a}, R^{2b}, R³, m, n, o, p, and Z are as defined in Table 7, or a pharmaceutically acceptable salt or solvate thereof.

TABLE 7
IX

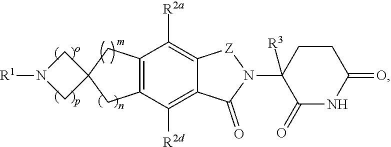

| Cpd. | | | | | | | | | |
|---|---|---|---|---|---|---|---|---|---|
| No. | R¹ | R^{2a} | R^{2d} | R³ | m | n | o | p | Z |
| 826 | —H | —H | —H | —H | 1 | 1 | 1 | 1 | —CH₂— |
| 827 | —H | —H | —H | —H | 1 | 1 | 1 | 1 | —C(═O)— |
| 826a | —H | —H | —H | —H | 1 | 1 | 2 | 2 | —CH₂— |
| 827a | —H | —H | —H | —H | 1 | 1 | 2 | 2 | —C(═O)— |
| 860 | —H | —H | —H | —H | 1 | 1 | 1 | 2 | —C(═O)— |
| 861 | —H | —H | —H | —H | 1 | 1 | 2 | 3 | —C(═O)— |

In another embodiment, Compounds of the Disclosure are compounds of Formula XIV, wherein R^{1a}, R^{1b}, R^{2a}, R^{2b}, R³, m, n, and Z are as defined in Table 8, or a pharmaceutically acceptable salt or solvate thereof.

TABLE 8
XIV

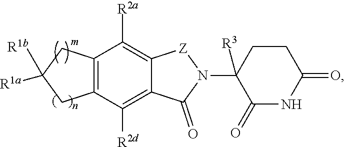

| Cpd. | | | | | | | | |
|---|---|---|---|---|---|---|---|---|
| No. | R^{1a} | R^{1b} | R^{2a} | R^{2d} | R³ | m | n | Z |
| 844 | —OH | —H | —H | —H | —H | 1 | 1 | —CH₂— |
| 845 | —CH₂OH | —H | —H | —H | —H | 1 | 1 | —CH₂— |
| 846 | —CHO | —H | —H | —H | —H | 1 | 1 | —CH₂— |
| 847 | —C(═O)OH | —H | —H | —H | —H | 1 | 1 | —CH₂— |
| 848 | —C(═O)— | | —H | —H | —H | 1 | 1 | —CH₂— |
| 849 | —OH | —H | —H | —H | —H | 1 | 1 | —C(═O)— |
| 850 | —CH₂OH | —H | —H | —H | —H | 1 | 1 | —C(═O)— |
| 851 | —CHO | —H | —H | —H | —H | 1 | 1 | —C(═O)— |
| 852 | —C(═O)OH | —H | —H | —H | —H | 1 | 1 | —C(═O)— |

| | | | | | | | |
|---|---|---|---|---|---|---|---|
| 853 | —C(═O)— | —H | —H | —H | 1 | 1 | —C(═O)— |

The present disclosure encompasses the preparation and use of salts of Compounds of the Disclosure. As used herein, the pharmaceutical “pharmaceutically acceptable salt” refers to salts or zwitterionic forms of Compounds of the Disclosure. Salts of Compounds of the Disclosure can be prepared during the final isolation and purification of the compounds or separately by reacting the compound with a suitable acid. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E.C. van Tonder et al., AAPS Pharm. Sci. Tech., 5(1):Article 12 (2004), and A.L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

### II. Intermediates of the Disclosure

The disclosure also provides synthetic intermediates, collectively referred to as

“Intermediates of the Disclosure,” that can be used to prepare Compounds of the Disclosure.

In one embodiment, Intermediates of the Disclosure are compounds of Formula VI:

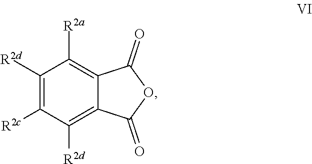

wherein R¹ is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷; and R^{2a}, R^{2b}, R^{2c}, R^{2d}, R⁴, R⁵, R⁶, and R⁷ are as defined in connection with Formula I.

In another embodiment, Intermediates of the Disclosure are compounds of Formula VII:

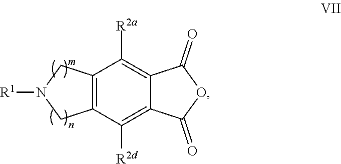

wherein R¹ is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷; and R^{2a} ,R^{2d}, m, n, R⁴, R⁵, R⁶, and R⁷ are as defined in connection with Formula II.

In another embodiment, Intermediates of the Disclosure are compounds of of Formula VIII:

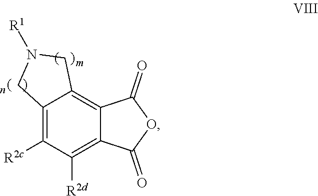

wherein R¹ is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷; and R^{2c}, R^{2a}, R^{2d}, m, n, R⁴, R⁵, R⁶, and R⁷ are as defined in connection with Formula III.

In another embodiment, Intermediates of the Disclosure are compounds of Formula XVII:

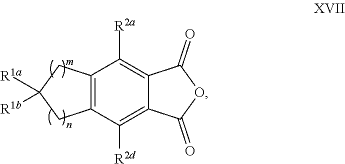

wherein R^{1a}, R^{1b}, R^{2a}, R^{2d}, R³, m, and n are as defined in connection with Formula XIV.

### III. Methods of Preparing Compounds of the Disclosure

The disclosure also provides methods of preparing Compounds of the Disclosure.

In one embodiment, the disclosure provides a method of making a compound of Formula I, wherein Z is —C(═O)—, the method comprising:

(i) reacting a compound of Formula V:

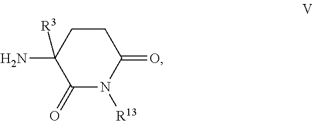

or a salt, e.g., HCl salt, thereof, wherein R³ is as defined in connection with Formula I;

with compound of Formula VI:

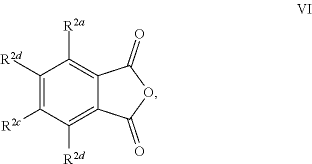

in a solvent, wherein R¹ is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷; and R^{2a}, R^{2b}, R^{2c}, R^{2d} , R⁴, R⁵, R⁶, and R⁷ are as defined in connection with Formula I.

In another embodiment, the disclosure provides a method of making a compound of Formula II, wherein Z is —C(═O)—, the method comprising:

(i) reacting a compound of Formula V:

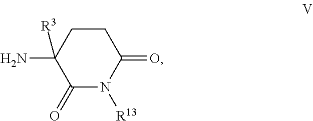

or a salt, e.g., HCl salt, thereof, wherein R³ is as defined in connection with Formula I;

with compound of Formula VII:

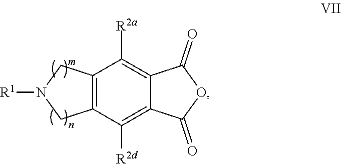

in a solvent, wherein R¹ is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy) alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷; and R^{2a}, R^{2d}, m, n, R⁴, R⁵, R⁶, and R⁷ are as defined in connection with Formula II.

In another embodiment, the disclosure provides a method of making a compound of Formula III, wherein Z is —C(═O)—, the method comprising:

(i) reacting a compound of Formula V:

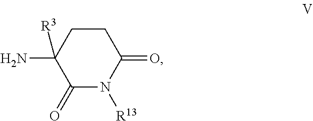

or a salt, e.g., HCl salt, thereof, wherein R³ is as defined in connection with Formula I;

with compound of Formula VIII:

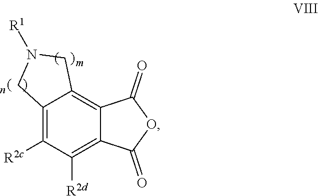

in a solvent, wherein R¹ is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy) alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷; and R^{2c}, R^{2a}, R^{2d}, m, n, R⁴, R⁵, R⁶, and R⁷ are as defined in connection with Formula III.

In another embodiment, R¹ is —C(═O)R⁴ and R⁴ is —OR^{4b}. In another embodiment, R^{4b} is C₁-C₄ alkyl.

In another embodiment, the solvent is selected from the group consisting of toluene, benzene, xylene, tetrahydrofuran (THF), dioxane, dimethylformamide (DMF), dimethylacetamide (DMA), N-methyl-2-pyrrolidone (NMP), dimethylsulfoxide (DMSO), acetic acid, and acetonitrile.

In another embodiment, Formula V is reacted with Formula VI at a temperature of about 40° C. to about 150° C. In another embodiment, Formula V is reacted with Formula VI at about 40° C. In another embodiment, Formula V is reacted with Formula VI at about 50° C. In another embodiment, Formula V is reacted with Formula VI at about 60° C. In another embodiment, Formula V is reacted with Formula VI at about 70° C. In another embodiment, Formula V is reacted with Formula VI at about 80° C. In another embodiment, Formula V is reacted with Formula VI at about 90° C. In another embodiment, Formula V is reacted with Formula VI at about 100° C. In another embodiment, Formula V is reacted with Formula VI at about 110° C. In another embodiment, Formula V is reacted with Formula VI at about 120° C. In another embodiment, Formula V is reacted with Formula VI at about 130° C. In another embodiment, Formula V is reacted with Formula VI at about 140° C. In another embodiment, Formula V is reacted with Formula VI at about 150° C.

### IV. Methods of Treating Disease with Compounds of the Disclosure and PROTAC Molecules

Compounds of the Disclosure inhibit CRBN ubiquitination and are thus useful in the treatment or prevention of a variety of diseases and conditions. In particular, Compounds of the Disclosure are useful in methods of treating or preventing a disease or condition wherein inhibition of CRBN ubiquitination provides a benefit. Foremost among these diseases and conditions are cancers and proliferative diseases. In one embodiment, such a cancer is referred to as a “CRBN-mediated cancer.” CRBN-mediated cancers are known in the art. The therapeutic methods of this disclosure comprise administering a therapeutically effective amount of a Compound of the Disclosure to a subject, e.g., human, in need thereof. The present methods also encompass optionally administering an optional therapeutic agent to the subject in addition to the Compound of the Disclosure. The optional therapeutic agent is selected from drugs known as useful in treating the disease or condition afflicting the subject in need thereof, e.g., a chemotherapeutic agent and/or radiation known as useful in treating a particular cancer.

In another embodiment, the present disclosure relates to a method of treating an individual suffering from a disease or condition wherein inhibition of CRBN ubiquitination provides a benefit, the method comprising administering a therapeutically effective amount of a Compound of the Disclosure.

Since Compounds of the Disclosure inhibit CRBN ubiquitination, a number of diseases and conditions mediated by CRBN ubiquitination can be treated by employing these compounds. The present disclosure is thus directed generally to a method for treating a condition or disorder responsive to inhibition of CRBN ubiquitination in a subject, e.g., a human subject, suffering from, or at risk of suffering from, a condition or disorder, e.g., cancer or inflammatory disease, the method comprising administering to the subject an effective amount of one or more Compounds of the Disclosure.

In another embodiment, the present disclosure is directed to a method of inhibiting CRBN ubiquitination in a subject in need thereof, said method comprising administering to the subject an effective amount of at least one Compound of the Disclosure.

The methods of the present disclosure can be accomplished by administering a Compound of the Disclosure or PROTAC Molecule as the neat compound or as a pharmaceutical composition. Administration of a pharmaceutical composition, or neat compound of a Compound of the Disclosure or PROTAC Molecule, can be performed during or after the onset of the disease or condition of interest. Typically, the pharmaceutical compositions are sterile, and contain no toxic, carcinogenic, or mutagenic compounds that would cause an adverse reaction when administered. Further provided are kits comprising a Compound of the Disclosure and, optionally, an optional therapeutic agent, packaged separately or together, and an insert having instructions for using these active agents.

In one embodiment, a Compound of the Disclosure is administered in conjunction with an optional therapeutic agent useful in the treatment of a disease or condition wherein inhibition of CRBN ubiquitination provides a benefit. The optional therapeutic agent is different from the Compound of the Disclosure. A Compound of the Disclosure and the optional therapeutic agent can be administered simultaneously or sequentially to achieve the desired effect. In addition, the Compound of the Disclosure and optional therapeutic agent can be administered from a single composition or two separate compositions. Likewise, in another embodiment, a PROTAC Molecule is administered in conjunction with an optional therapeutic agent.

The optional therapeutic agent is administered in an amount to provide its desired therapeutic effect. The effective dosage range for each optional therapeutic agent is known in the art, and the optional therapeutic agent is administered to an individual in need thereof within such established ranges.

A Compound of the Disclosure or PROTAC Molecule and the optional therapeutic agent can be administered together as a single-unit dose or separately as multi-unit doses, wherein the Compound of the Disclosure or PROTAC Molecule is administered before the optional therapeutic agent or vice versa. One or more doses of the Compound of the Disclosure and/or one or more dose of the optional therapeutic agent can be administered. The Compound of the Disclosure or PROTAC Molecule therefore can be used in conjunction with one or more optional therapeutic agents, for example, but not limited to, anticancer agents.

Diseases and conditions treatable by the methods of the present disclosure include, but are not limited to, cancer and other proliferative disorders, or an inflammatory disease. In one embodiment, a human subject is treated with a Compound of the Disclosure, or a pharmaceutical composition comprising a Compound of the Disclosure, wherein the compound is administered in an amount sufficient to inhibit CRBN ubiquitination in the subject.

In another aspect, the present disclosure provides a method of treating cancer in a subject comprising administering a therapeutically effective amount of a Compound of the Disclosure. While not being limited to a specific mechanism, in some embodiments, Compounds of the Disclosure treat cancer by inhibiting CRBN ubiquitination.

In another aspect, the present disclosure provides a method of treating cancer in a subject comprising administering a therapeutically effective amount of a PROTAC Molecule to the subject.

Examples of treatable cancers include, but are not limited to, any one or more of the cancers of Table 4.

TABLE 4

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic |
| lymphoma | rhabdomyosarcoma | sarcoma | fibroma |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial | |
| | | tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular | follicular thyroid | ganglioneuroma | gastrointestinal |
| lymphoma | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological | hepatoblastoma |
| cancer | | cancer | |
| hepatosplenic T-cell | Hodgkin's | non-Hodgkin's | invasive lobular |
| lymphoma | lymphoma | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic |
| | leukemia | leukemia | lymphocytic |
| | | | leukemia |
| liver cancer | small cell lung | non-small cell lung | MALT lymphoma |
| | cancer | cancer | |
| malignant fibrous | malignant peripheral | malignant triton | mantle cell |
| histiocytoma | nerve sheath tumor | tumor | lymphoma |
| marginal zone B- | mast cell leukemia | mediastinal germ | medullary |
| cell lymphoma | | cell tumor | carcinoma of the |
| | | | breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid | myxoma | myxosarcoma | nasopharyngeal |
| liposarcoma | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell |
| carcinoma | | tumors | carcinoma |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial |
| | | | carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In another embodiment, the cancer is a solid tumor. In another embodiment, the cancer a hematological cancer. Exemplary hematological cancers include, but are not limited to, the cancers listed in Table 5. In another embodiment, the hematological cancer is acute lymphocytic leukemia, chronic lymphocytic leukemia (including B-cell chronic lymphocytic leukemia), or acute myeloid leukemia. In another embodiment, the hematological cancer is multiple myeloma.

TABLE 5

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In another embodiment, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In another embodiment, the cancer is selected from the group consisting of acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

In another embodiment, the present disclosure provides a method of treating a benign proliferative disorder, such as, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

In another embodiment, the present disclosure provides a method of treating an inflammatory disease. For example, Compounds of the Disclosure can be used to treat infectious and noninfectious inflammatory events and autoimmune and other inflammatory diseases by administration of a therapeutically effective amount to a subject, in particular a human in need of such treatment. Examples of autoimmune and inflammatory diseases, disorders, and syndromes treated using the compounds and methods described herein include inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, Type I diabetes, septic shock, lupus, e.g., cutaneous lupus, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituatarism, Guillain-Barre syndrome, Behcet's disease, scleracierma, mycosis fungoides, acute inflammatory responses (such as acute respiratory distress syndrome and ischemia/reperfusion injury), and Graves' disease.

In another embodiment, the present disclosure provides a therapeutic method of modulating CRBN ubiquitination in vivo in diseases mentioned above, in particular cancer, by administering a therapeutically effective amount of a Compound of the Disclosure to a subject in need of such therapy.

In methods of the present disclosure, a therapeutically effective amount of a Compound of the Disclosure or PROTAC Molecule, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure or PROTAC Molecule can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure or PROTAC Molecule is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure or PROTAC Molecule that is sufficient to maintain therapeutic effects.

Toxicity and therapeutic efficacy of the Compounds of the Disclosure or the PROTAC Molecules can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure or PROTAC Molecule required for use in therapy varies with the nature of the condition being treated, the length of time that activity is desired, and the age and the condition of the subject, and ultimately is determined by the attendant physician. Dosage amounts and intervals can be adjusted individually to provide plasma levels of the Compound of the Disclosure that are sufficient to maintain the desired therapeutic effects. The desired dose can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure or PROTAC Molecule used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure or PROTAC Molecule can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

The dosage of a composition containing a Compound of the Disclosure or PROTAC Molecule, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual subject, which can vary with the age, weight, and response of the particular subject.

Compounds of the Disclosure and PROTAC Molecules typically are administered in admixture with a pharmaceutical carrier to give a pharmaceutical composition selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure or PROTAC Molecule.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure or PROTAC Molecule. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure or PROTAC Molecule.

When a therapeutically effective amount of a Compound of the Disclosure or PROTAC Molecule is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure or PROTAC Molecules can be readily combined with pharmaceutically acceptable carriers well-known in the art. Standard pharmaceutical carriers are described in Remington's Pharmaceutical Sciences, Mack Publishing Co., Easton, Pa., 19th ed. 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Compounds of the Disclosure or PROTAC Molecules can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure or PROTAC Molecule can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure or PROTAC Molecules also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure or PROTAC Molecules can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compounds of the Disclosure or PROTAC Molecules also can be injected parenterally, for example, intravenously, intramuscularly, subcutaneously, or intracoronarily. For parenteral administration, the Compounds of the Disclosure or PROTAC Molecules are typically used in the form of a sterile aqueous solution which can contain other substances, for example, salts or monosaccharides, such as mannitol or glucose, to make the solution isotonic with blood.

### V. Optional Therapeutic Agents

In some therapeutic methods and uses of the disclosure, a Compound of the Disclosure or PROTAC Molecule is administered to a subject having a disease, disorder, or condition, e.g., cancer, as a single agent. In other therapeutic methods and uses of the disclosure, a Compound of the Disclosure or PROTAC Molecule is administered to a subject having a disease, disorder, or condition, e.g., cancer, in combination with one or more optional therapeutic agents. In one embodiment, a Compound of the Disclosure or PROTAC Molecule is administered in combination with one optional therapeutic agent. In another embodiment, a Compound of the Disclosure or PROTAC Molecule is administered in combination with two optional therapeutic agents. In another embodiment, a Compound of the Disclosure or PROTAC Molecule is administered in combination with three optional therapeutic agents. Optional therapeutic agents useful in treating cancer patients include those known in the art as well as those developed in the future.

Optional therapeutic agents are administered in an amount to provide their desired therapeutic effect. The effective dosage range for each optional therapeutic agent is known in the art, and the optional therapeutic agent is administered to an individual in need thereof within such established ranges.

A Compound of the Disclosure or PROTAC Molecule and the optional therapeutic agent(s) can be administered together as a single-unit dose or separately as multi-unit doses, and in any order, e.g., wherein a Compound of the Disclosure is administered before the optional therapeutic agent(s), or vice versa. One or more doses of a Compound of the Disclosure or PROTAC Molecule and the optional therapeutic agent(s) can be administered to the subject.

In one embodiment, the optional therapeutic agent is an immune checkpoint inhibitor. Immune checkpoint inhibitors are therapies that blockade immune system inhibitor checkpoints. Immune checkpoints can be stimulatory or inhibitory. Blockade of inhibitory immune checkpoint activates immune system function and can be used for cancer immunotherapy. Pardoll, Nature Reviews. Cancer 12:252-64 (2012). Tumor cells turn off activated T cells when they attach to specific T-cell receptors. Immune checkpoint inhibitors prevent tumor cells from attaching to T cells, which results in T cells remaining activated. In effect, the coordinated action by cellular and soluble components combats pathogens and injuries by cancers. The modulation of immune system pathways may involve changing the expression or the functional activity of at least one component of the pathway to then modulate the response by the immune system. U.S. 2015/0250853. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in one embodiment, the immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In another embodiment, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, pidilzumab, and cemiplimab-rwlc. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1 . Particular anti-PD-L1 antibodies include, but are not limited to, avelumab, atezolizumab, durvalumab, and BMS-936559. For a general discussion of the availability, methods of production, mechanism of action, and clinical studies, see U.S. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{c}1 T-cell responses. The TIM3 pathway is considered a target for anticancer immunotherapy due to its expression on dysfunctional CD8⁺T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See Unanue, E.R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053 and have the structure:

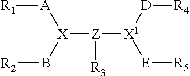

or a pharmaceutically acceptable salt thereof, wherein the compound comprises at least 5 amino acids useful as therapeutic agents capable of inhibiting the PD-1 signaling pathway.

Another class of immune checkpoint inhibitors include inhibitors of certain metabolic enzymes, such as indoleamine 2,3 dioxygenase (IDO), which is expressed by infiltrating myeloid cells and tumor cells, and isocitrate dehydrogenase (IDH), which is mutated in leukemia cells. Mutants of the IDH enzyme lead to increased levels of 2-hydroxyglutarate (2-HG), which prevent myeloid differentiation. Stein et al.,Blood 130:722-31 (2017); Wouters, Blood 130:693-94 (2017). Particular mutant IDH blocking agents include, but are not limited to, ivosidenib and enasidenib mesylate. Dalle and DiNardo, Ther Adv Hematol 9(7):163-73 (2018); Nassereddine et al., Onco Targets Ther 12:303-08 (2018). The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to, levo-1-methyl typtophan (L-1MT) and 1-methyl-tryptophan (1MT). Qian et al., Cancer Res 69:5498-504 (2009); and Löb et al., Cancer Immunol Immunother 58:153-7 (2009).

In one embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

In another embodiment, the optional therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat and panobinostat lactate.

In another embodiment, the optional therapeutic agent is a chemotherapeutic agent or other anti-proliferative agent that can be administered in combination with a Compound of the Disclosure to treat cancer. Examples of conventional therapies and anticancer agents that can be used in combination with a Compound of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved biologic therapy or chemotherapy, e.g., a treatment regimen that uses drugs to stop the growth of cancer cells, either by killing the cells or by stopping them from dividing. Chemotherapy may be given by mouth, injection, or infusion, or on the skin, depending on the type and stage of the cancer being treated.

Nonlimiting exemplary antiproliferative compounds include an aromatase inhibitor; an anti-estrogen; an anti-androgen; a gonadorelin agonist; a topoisomerase I inhibitor; a topoisomerase II inhibitor; a microtubule active agent; an alkylating agent, e.g., temozolomide; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include tamoxifen, fulvestrant, raloxifene, and raloxifene hydrochloride. Anti-androgens include, but are not limited to, bicalutamide and apalutamide. Gonadorelin agonists include, but are not limited to, abarelix, goserelin, and goserelin acetate.

Nonlimiting exemplary topoisomerase I inhibitors include topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etopo side and tenipo side.

Microtubule active agents include microtubule stabilizing, microtubule destabilizing compounds, and microtubulin polymerization inhibitors including, but not limited to, taxanes, such as paclitaxel and docetaxel; discodermolides; cochicine and epothilones and derivatives thereof.

Nonlimiting exemplary alkylating agents include cyclophosphamide, ifosfamide, melphalan, trabectedin, and nitrosoureas, such as carmustine and lomustine.

Nonlimiting exemplary matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Nonlimiting exemplary mTOR inhibitors include compounds that inhibit the mammalian target of rapamycin (mTOR) and possess antiproliferative activity such as sirolimus, everolimus, CCI-779, and ABT578.

Nonlimiting exemplary antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Nonlimiting exemplary platin compounds include carboplatin, cis-platin, cisplatinum, and oxaliplatin.

Nonlimiting exemplary methionine aminopeptidase inhibitors include bengamide or a derivative thereof and PPI-2458.

Nonlimiting exemplary bisphosphonates include etridonic acid, clodronic acid, tiludronic acid, pamidronic acid, alendronic acid, ibandronic acid, risedronic acid, and zoledronic acid.

Nonlimiting exemplary heparanase inhibitors include compounds that target, decrease, or inhibit heparin sulfate degradation, such as PI-88 and OGT2115.

Nonlimiting exemplary compounds which target, decrease, or inhibit the oncogenic activity of Ras include farnesyl transferase inhibitors, such as L-744832, DK8G557, tipifarnib, and lonafarnib.

Nonlimiting exemplary telomerase inhibitors include compounds that target, decrease, or inhibit the activity of telomerase, such as compounds that inhibit the telomerase receptor, such as telomestatin.

Nonlimiting exemplary proteasome inhibitors include compounds that target, decrease, or inhibit the activity of the proteasome including, but not limited to, bortezomib. In some embodiments, the proteasome inhibitor is carfilzomib or ixazomib.

Nonlimiting exemplary FMS-like tyrosine kinase inhibitors, which are compounds targeting, decreasing or inhibiting the activity of FMS-like tyrosine kinase receptors (Flt-3R), include gilteritinib, interferon, I-β-D-arabinofuransylcytosine (ara-c), and bisulfan; and ALK inhibitors, which are compounds that target, decrease, or inhibit anaplastic lymphoma kinase, include alectinib, brigatinib, and lorlatinib.

Nonlimiting exemplary Flt-3 inhibitors include PKC412, midostaurin, a staurosporine derivative, SU11248, MLN518, and gilteritinib.

Nonlimiting exemplary HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino,17-demethoxygeldanamycin (17AAG), a geldanamycin derivative; other geldanamycin related compounds; radicicol and HDAC inhibitors.

Nonlimiting exemplary protein tyrosine kinase and/or serine and/or threonine kinase inhibitors or lipid kinase inhibitors, include a) a compound targeting, decreasing, or inhibiting the activity of the platelet-derived growth factor-receptors (PDGFR), such as a compound that targets, decreases, or inhibits the activity of PDGFR, including olaratumab and N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR), such as erdafitinib and lenvatinib; c) a compound targeting, decreasing, or inhibiting the activity of the insulin-like growth factor receptor I (IGF-IR), such as brigatinib; d) a compound targeting, decreasing, or inhibiting the activity of the vascular endothelial growth factor-receptors (VEGFR), such as lenvatinib; e) a compound targeting, decreasing, or inhibiting the activity of the Trk receptor tyrosine kinase family, or ephrin B4 inhibitors, such as larotrectinib; f) a compound targeting, decreasing, or inhibiting the activity of the Axl receptor tyrosine kinase family; g) a compound targeting, decreasing, or inhibiting the activity of the Ret receptor tyrosine kinase, such as alectinib; h) a compound targeting, decreasing, or inhibiting the activity of the Kit/SCFR receptor tyrosine kinase, such as imatinib; i) a compound targeting, decreasing, or inhibiting the activity of the c-Kit receptor tyrosine kinases, such as imatinib; j) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; k) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; abemaciclib; binimetinib; cobimetinib; encorafenib; neratinib; palbociclib; ribociclib; 1) a compound targeting, decreasing or inhibiting the activity of a protein-tyrosine kinase, such as acalabrutinib, imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); m) a compound targeting, decreasing, or inhibiting the activity of the epidermal growth factor family of receptor tyrosine kinases (EGFR, ErbB2, ErbB3, ErbB4 as homo- or heterodimers) and their mutants, such as brigatinib, CP 358774, ZD 1839, ZM 105180; trastuzumab, cetuximab, gefitinib, erlotinib, osimertinib, dacomitinib, necitumumab, neratinib, OSI-774, Cl-1033, EKB-569, GW-2016, antibodies El.l, E2.4, E2.5, E6.2, E6.4, E2.11, E6.3 and E7.6.3, and 7H-pyrrolo-[2,3-d]pyrimidine derivatives; n) a compound targeting, decreasing or inhibiting the activity of a phosphatidylinositol 3-kinase (PI3K), such as alpelisib, copanlisib, and duvelisib; and o) a compound targeting, decreasing, or inhibiting the activity of the c-Met receptor.

Nonlimiting exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a Compound of the Disclosure include: avastin, daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

A number of suitable optional therapeutic, e.g., anticancer, agents are contemplated for use in the therapeutic methods provided herein. Indeed, the methods provided herein can include, but are not limited to, administration of numerous optional therapeutic agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of optional therapeutic agents such as chemotherapeutic compounds and anticancer therapies suitable for co-administration with the disclosed compounds are known to those skilled in the art.

In certain embodiments, anticancer agents comprise agents that induce or stimulate apoptosis. Agents that induce or stimulate apoptosis include, for example, agents that interact with or modify DNA, such as by intercalating, cross-linking, alkylating, or otherwise damaging or chemically modifying DNA. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor). Additional anticancer agents include: vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, apalutamide, bicalutamide, finasteride, aminoglutethamide, ketoconazole, and corticosteroids); BCL-2 inhibitors (e.g., venetoclax); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTAS ONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the therapeutic methods provided herein include administering to a subject having cancer (a cancer patient) therapeutically effective amounts of a Compound of the Disclosure, an immune checkpoint inhibitor, and at least one additional optional therapeutic agent, e.g., an anti-hyperproliferative or antineoplastic agent selected from alkylating agents, antimetabolites, and natural products (e.g., herbs and other plant and/or animal derived compounds).

Alkylating agents suitable for use in the present methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the therapeutic methods of the present disclosure. For example, the U.S. Food and Drug Administration (FDA) maintains a formulary of oncolytic agents approved for use in the United States. International counterpart agencies to the FDA maintain similar formularies. Those skilled in the art will appreciate that the “product labels” required on all U.S. approved chemotherapeutics describe approved indications, dosing information, toxicity data, and the like, for the exemplary agents.

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calaspargase pegol-mknl, calcitriol, CCI-779, CDB-2914, cefixime, cetuximab, CG0070, cilengitide, clofarabine, combretastatin A4 phosphate, CP-675,206, CP-724,714, CpG 7909, curcumin, daratumumab, decitabine, DENSPM, dinutuximab, doxercalciferol, E7070, E7389, ecteinascidin 743, efaproxiral, eflornithine, EKB-569, elotuzumab, enzastaurin, erlotinib, exisulind, fenretinide, flavopiridol, fludarabine, flutamide, fotemustine, FR901228, G17DT, galiximab, gefitinib, genistein, glasdegib, glufosfamide, GTI-2040, histrelin, HKI-272, homoharringtonine, HSPPC-96, hu14.18-interleukin-2 fusion protein, HuMax-CD4, iloprost, imiquimod, infliximab, inotuzumab ozogamicin, interleukin-12, IPI-504, irofulven, ixabepilone, lapatinib, lenalidomide, lestaurtinib, leuprolide, LMB -9 immunotoxin, lonafarnib, luniliximab, lutetium Lu 177 dotatate, mafosfamide, MB07133, MDX-010, MLN2704, mogamulizumab-kpkc, monoclonal antibody 3F8, monoclonal antibody J591, motexafin, moxetumomab pasudotox-tdfk, MS-275, MVA-MUC1-IL2, nilutamide, niraparib, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, polatuzumab vedotin-piiq, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, rucaparib, S-1, S-8184, satraplatin, SB-, 15992, SGN-0010, SGN-40, sonidegib, sorafenib, SR31747A, ST1571, SU011248, suberoylanilide hydroxamic acid, suramin, tagraxofusp-erzs, talabostat, talampanel, talazoparib, tariquidar, temsirolimus, TGFa-PE38 immunotoxin, thalidomide, thymalfasin, tipifarnib, tirapazamine, TLK286, trabectedin, trifluridine and tipiracil hydrochloride, trimetrexate glucuronate, TroVax, UCN-1, valproic acid, vinflunine, VNP40101 M , volociximab, vorinostat, VX-680, ZD1839, ZD6474, zileuton, and zosuquidar trihydrochloride.

In one embodiment, the optional therapeutic agent comprises one of the anti-cancer drugs or anti-cancer drug combinations listed in Table 6.

TABLE 6

| | | | |
|---|---|---|---|
| Abemaciclib | Abiraterone | Abraxane (Paclitaxel | ABVD |
| | Acetate | Albumin-stabilized | |
| | | Nanoparticle | |
| | | Formulation) | |
| ABVE | ABVE-PC | AC | Acalabrutinib |
| AC-T | Actemra | Adcetris (Brentuximab | ADE |
| | (Tocilizumab) | Vedotin) | |
| Ado-Trastuzumab | Adriamycin | Afatinib Dimaleate | Afinitor |
| Emtansine | (Doxorubicin | | (Everolimus) |
| | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta (Pemetrexed | Aliqopa |
| | | Disodium) | (Copanlisib |
| | | | Hydrochloride) |
| Alkeran for | Alkeran Tablets | Aloxi (Palonosetron | Alunbrig |
| Injection | (Melphalan) | Hydrochloride) | (Brigatinib) |
| (Melphalan | | | |
| Hydrochloride) | | | |
| Ameluz | Amifostine | Aminolevulinic Acid | Anastrozole |
| (Aminolevulinic | | | |
| Acid) | | | |
| Apalutamide | Aprepitant | Aranesp (Darbepoetin | Aredia |
| | | Alfa) | (Pamidronate |
| | | | Disodium) |
| Arimidex | Aromasin | Arranon (Nelarabine) | Arsenic Trioxide |
| (Anastrozole) | (Exemestane) | | |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (Iobenguane I 131) | (Avelumab) | | (Belinostat) |
| Belinostat | Bendamustine | Bendeka (Bendamustine | BEP |
| | Hydrochloride | Hydrochloride) | |
| Besponsa | Bevacizumab | Bexarotene | Bicalutamide |
| (Inotuzumab | | | |
| Ozogamicin) | | | |
| BiCNU | Binimetinib | Bleomycin | Blinatumomab |
| (Carmustine) | | | |
| Blincyto | Bortezomib | Bosulif (Bosutinib) | Bosutinib |
| (Blinatumomab) | | | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib-S- |
| | | | Malate) |
| Cabozantinib-S- | CAF | Calquence | Campath |
| Malate | | (Acalabrutinib) | (Alemtuzumab) |
| Camptosar | Capecitabine | CAPOX | Carac |
| (Irinotecan | | | (Fluorouracil-- |
| Hydrochloride) | | | Topical) |
| Carboplatin | CARBOPLATIN- | Carfilzomib | Carmustine |
| | TAXOL | | |
| Carmustine | Casodex | CEM | Cemiplimab-rwlc |
| Implant | (Bicalutamide) | | |
| Ceritinib | Cerubidine | Cervarix (Recombinant | Cetuximab |
| | (Daunorubicin | HPV Bivalent Vaccine) | |
| | Hydrochloride) | | |
| CEV | Chlorambucil | CHLORAMBUCIL- | CHOP |
| | | PREDNISONE | |
| Cisplatin | Cladribine | Clofarabine | Clolar |
| | | | (Clofarabine) |
| CMF | Cobimetinib | Cometriq (Cabozantinib- | Copanlisib |
| | | S-Malate) | Hydrochloride |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclophosphamide | Cyramza | Cytarabine | Cytarabine |
| | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin Alfa |
| Darzalex | Dasatinib | Daunorubicin | Daunorubicin |
| (Daratumumab) | | Hydrochloride | Hydrochloride |
| | | | and Cytarabine |
| | | | Liposome |
| Decitabine | Defibrotide | Defitelio (Defibrotide | Degarelix |
| | Sodium | Sodium) | |
| Denileukin | Denosumab | DepoCyt (Cytarabine | Dexamethasone |
| Diftitox | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL (Doxorubicin | Durvalumab |
| Hydrochloride | Hydrochloride | Hydrochloride | |
| | Liposome | Liposome) | |
| Duvelisib | Efudex | Eligard (Leuprolide | Elitek |
| | (Fluorouracil-- | Acetate) | (Rasburicase) |
| | Topical) | | |
| Ellence | Elotuzumab | Eloxatin (Oxaliplatin) | Eltrombopag |
| (Epirubicin | | | Olamine |
| Hydrochloride) | | | |
| Emend | Empliciti | Enasidenib Mesylate | Encorafenib |
| (Aprepitant) | (Elotuzumab) | | |
| Enzalutamide | Epirubicin | EPOCH | Epoetin Alfa |
| | Hydrochloride | | |
| Epogen (Epoetin | Erbitux | Eribulin Mesylate | Erivedge |
| Alfa) | (Cetuximab) | | (Vismodegib) |
| Erleada | Erlotinib | Erwinaze (Asparaginase | Ethyol |
| (Apalutamide) | Hydrochloride | Erwinia chrysanthemi) | (Amifostine) |
| Etopophos | Etoposide | Etoposide Phosphate | Evacet |
| (Etoposide | | | (Doxorubicin |
| Phosphate) | | | Hydrochloride |
| | | | Liposome) |
| Everolimus | Evista (Raloxifene | Evomela (Melphalan | Exemestane |
| | Hydrochloride) | Hydrochloride) | |
| 5-FU | 5-FU | Fareston (Toremifene) | Farydak |
| (Fluorouracil | (Fluorouracil-- | | (Panobinostat |
| Injection) | Topical) | | lactate) |
| Faslodex | FEC | Femara (Letrozole) | Filgrastim |
| (Fulvestrant) | | | |
| Firmagon | Fludarabine | Fluoroplex (Fluorouracil-- | Fluorouracil |
| (Degarelix) | Phosphate | Topical) | Injection |
| Fluorouracil-- | Flutamide | FOLFIRI | FOLFIRI- |
| Topical | | | BEVACIZUMAB |
| FOLFIRI- | FOEFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva (Obinutuzumab) | Gefitinib |
| (Recombinant | (Recombinant | | |
| HPV Quadrivalent | HPV Nonavalent | | |
| Vaccine) | Vaccine) | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif (Afatinib | Gleevec (Imatinib | Gliadel Wafer |
| (Gemcitabine | Dimaleate) | Mesylate) | (Carmustine |
| Hydrochloride) | | | Implant) |
| Glucarpidase | Goserelin Acetate | Granisetron | Granisetron |
| | | | Hydrochloride |
| Granix | Halaven (Eribulin | Hemangeol (Propranolol | Herceptin |
| (Filgrastim) | Mesylate) | Hydrochloride) | (Trastuzumab) |
| HPV Bivalent | HPV Nonavalent | HPV Quadrivalent | Hycamtin |
| Vaccine, | Vaccine, | Vaccine, Recombinant | (Topotecan |
| Recombinant | Recombinant | | Hydrochloride) |
| Hydrea | Hydroxyurea | Hyper-CVAD | Ibrance |
| (Hydroxyurea) | | | (Palbociclib) |
| Ibritumomab | Ibrutinib | ICE | Iclusig (Ponatinib |
| Tiuxetan | | | Hydrochloride) |
| Idarubicin | Idelalisib | Idhifa (Enasidenib | Ifex (Ifosfamide) |
| Hydrochloride | | Mesylate) | |
| Ifosfamide | IL-2 | Imatinib Mesylate | Imbruvica |
| | (Aldesleukin) | | (Ibrutinib) |
| Imfinzi | Imiquimod | Imlygic (Talimogene | Inlyta (Axitinib) |
| (Durvalumab) | | Laherparepvec) | |
| Inotuzumab | Interferon Alfa- | Interleukin-2 | Intron A |
| Ozogamicin | 2b, Recombinant | (Aldesleukin) | (Recombinant |
| | | | Interferon Alfa- |
| | | | 2b) |
| Iobenguane I 131 | Ipilimumab | Iressa (Gefitinib) | Irinotecan |
| | | | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi (Ruxolitinib | JEB |
| | (Ixabepilone) | Phosphate) | |
| Jevtana | Kadcyla (Ado- | Kepivance (Palifermin) | Keytruda |
| (Cabazitaxel) | Trastuzumab | | (Pembrolizumab) |
| | Emtansine) | | |
| Kisqali | Kymriah | Kyprolis (Carfilzomib) | Lanreotide |
| (Ribociclib) | (Tisagenlecleucel) | | Acetate |
| Lapatinib | Larotrectinib | Lartruvo (Olaratumab) | Lenalidomide |
| Ditosylate | Sulfate | | |
| Lenvatinib | Lenvima | Letrozole | Leucovorin |
| Mesylate | (Lenvatinib | | Calcium |
| | Mesylate) | | |
| Leukeran | Leuprolide | Levulan | Libtayo |
| (Chlorambucil) | Acetate | Kerastik (Aminolevulinic | (Cemiplimab- |
| | | Acid) | rwlc) |
| LipoDox | Lomustine | Lonsurf (Trifluridine and | Lorbrena |
| (Doxorubicin | | Tipiracil Hydrochloride) | (Lorlatinib) |
| Hydrochloride | | | |
| Liposome) | | | |
| Lorlatinib | Lumoxiti | Lupron (Leuprolide | Lupron Depot |
| | (Moxetumomab | Acetate) | (Leuprolide |
| | Pasudotox-tdfk) | | Acetate) |
| Lutathera | Lutetium (Lu 177- | Lynparza (Olaparib) | Marqibo |
| (Lutetium Lu 177- | Dotatate) | | (Vincristine |
| Dotatate) | | | Sulfate |
| | | | Liposome) |
| Matulane | Mechlorethamine | Megestrol Acetate | Mekinist |
| (Procarbazine | Hydrochloride | | (Trametinib) |
| Hydrochloride) | | | |
| Mektovi | Melphalan | Melphalan | Mercaptopurine |
| (Binimetinib) | | Hydrochloride | |
| Mesna | Mesnex (Mesna) | Methotrexate | Methylnaltrexone |
| | | | Bromide |
| Midostaurin | Mitomycin C | Mitoxantrone | Mogamulizumab- |
| | | Hydrochloride | kpkc |
| Moxetumomab | Mozobil | Mustargen | MVAC |
| Pasudotox-tdfk | (Plerixafor) | (Mechlorethamine | |
| | | Hydrochloride) | |
| Myleran | Mylotarg | Nanoparticle Paclitaxel | Navelbine |
| (Busulfan) | (Gemtuzumab | (Paclitaxel Albumin- | (Vinorelbine |
| | Ozogamicin) | stabilized Nanoparticle | Tartrate) |
| | | Formulation) | |
| Necitumumab | Nelarabine | Neratinib Maleate | Nerlynx |
| | | | (Neratinib |
| | | | Maleate) |
| Netupitant and | Neulasta | Neupogen (Filgrastim) | Nexavar |
| Palonosetron | (Pegfilgrastim) | | (Sorafenib |
| Hydrochloride | | | Tosylate) |
| Nilandron | Nilotinib | Nilutamide | Ninlaro |
| (Nilutamide) | | | (Ixazomib |
| | | | Citrate) |
| Niraparib Tosylate | Nivolumab | Nplate (Romiplostim) | Obinutuzumab |
| Monohydrate | | | |
| Odomzo | OEPA | Ofatumumab | OFF |
| (Sonidegib) | | | |
| Olaparib | Olaratumab | Omacetaxine | Oncaspar |
| | | Mepesuccinate | (Pegaspargase) |
| Ondansetron | Onivyde | Ontak (Denileukin | Opdivo |
| Hydrochloride | (Irinotecan | Diftitox) | (Nivolumab) |
| | Hydrochloride | | |
| | Liposome) | | |
| OPPA | Osimertinib | Oxaliplatin | Paclitaxel |
| Paclitaxel | PAD | Palbociclib | Palifermin |
| Albumin-stabilized | | | |
| Nanoparticle | | | |
| Formulation | | | |
| Palonosetron | Palonosetron | Pamidronate Disodium | Panitumumab |
| Hydrochloride | Hydrochloride | | |
| | and Netupitant | | |
| Panobinostat | Pazopanib | PCV | PEB |
| Lactate | Hydrochloride | | |
| Pegaspargase | Pegfilgrastim | Peginterferon Alfa-2b | PEG-Intron |
| | | | (Peginterferon |
| | | | Alfa-2b) |
| Pembrolizumab | Pemetrexed | Perjeta (Pertuzumab) | Pertuzumab |
| | Disodium | | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab- | | |
| | kpkc) | | |
| Procarbazine | Procrit (Epoetin | Proleukin (Aldesleukin) | Prolia |
| Hydrochloride | Alfa) | | (Denosumab) |
| Promacta | Propranolol | Provenge (Sipuleucel-T) | Purinethol |
| (Eltrombopag | Hydrochloride | | (Mercaptopurine) |
| Olamine) | | | |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant | Recombinant | Recombinant Interferon | Regorafenib |
| Human | Human | Alfa-2b | |
| Papillomavirus | Papillomavirus | | |
| (HPV) Nonavalent | (HPV) | | |
| Vaccine | Quadrivalent | | |
| | Vaccine | | |
| Relistor | R-EPOCH | Retacrit (Epoetin Alfa) | Revlimid |
| (Methylnaltrexone | | | (Lenalidomide) |
| Bromide) | | | |
| Rheumatrex | Ribociclib | R-ICE | Rituxan |
| (Methotrexate) | | | (Rituximab) |
| Rituxan Hycela | Rituximab | Rituximab and | Rolapitant |
| (Rituximab and | | Hyaluronidase Human | Hydrochloride |
| Hyaluronidase | | | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt (Midostaurin) | Sancuso |
| Camsylate | Phosphate | | (Granisetron) |
| Sclerosol | Siltuximab | Sipuleucel-T | Somatuline Depot |
| Intrapleural | | | (Lanreotide |
| Aerosol (Talc) | | | Acetate) |
| Sonidegib | Sorafenib | Sprycel (Dasatinib) | STANFORD V |
| | Tosylate | | |
| Sterile Talc | Steritalc (Talc) | Stivarga (Regorafenib) | Sunitinib Malate |
| Powder (Talc) | | | |
| Sustol | Sutent (Sunitinib | Sylatron (Peginterferon | Sylvant |
| (Granisetron) | Malate) | Alfa-2b) | (Siltuximab) |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva (Erlotinib | Targretin (Bexarotene) | Tasigna |
| (Cytarabine) | Hydrochloride) | | (Nilotinib) |
| Tavalisse | Taxol (Paclitaxel) | Taxotere (Docetaxel) | Tecentriq |
| (Fostamatinib | | | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak (Fluorouracil-- | Topotecan |
| | | Topical) | Hydrochloride |
| Toremifene | Torisel | Totect (Dexrazoxane | TPF |
| | (Temsirolimus) | Hydrochloride) | |
| Trabectedin | Trametinib | Trastuzumab | Treanda |
| | | | (Bendamustine |
| | | | Hydrochloride) |
| Trexall | Trifluridine and | Trisenox (Arsenic | Tykerb (Lapatinib |
| (Methotrexate) | Tipiracil | Trioxide) | Ditosylate) |
| | Hydrochloride | | |
| Unituxin | Uridine Triacetate | VAC | Valrubicin |
| (Dinutuximab) | | | |
| Valstar | Vandetanib | VAMP | Varubi |
| (Valrubicin) | | | (Rolapitant |
| | | | Hydrochloride) |
| Vectibix | VeIP | Velcade (Bortezomib) | Vemurafenib |
| (Panitumumab) | | | |
| Venclexta | Venetoclax | Verzenio (Abemaciclib) | Vidaza |
| (Venetoclax) | | | (Azacitidine) |
| Vinblastine Sulfate | Vincristine | Vincristine Sulfate | Vinorelbine |
| | Sulfate | Liposome | Tartrate |
| VIP | Vismodegib | Vistogard (Uridine | Vitrakvi |
| | | Triacetate) | (Larotrectinib |
| | | | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda (Capecitabine) | XELIRI |
| (Daunorubicin | (Crizotinib) | | |
| Hydrochloride and | | | |
| Cytarabine | | | |
| Liposome) | | | |
| XELOX | Xgeva | Xofigo (Radium 223 | Xtandi |
| | (Denosumab) | Dichloride) | (Enzalutamide) |
| Yervoy | Yescarta | Yondelis (Trabectedin) | Zaltrap (Ziv- |
| (Ipilimumab) | (Axicabtagene | | Aflibercept) |
| | Ciloleucel) | | |
| Zarxio (Filgrastim) | Zejula (Niraparib | Zelboraf (Vemurafenib) | Zevalin |
| | Tosylate | | (Ibritumomab |
| | Monohydrate) | | Tiuxetan) |
| Zinecard | Ziv-Aflibercept | Zofran (Ondansetron | Zoladex |
| (Dexrazoxane | | Hydrochloride) | (Goserelin |
| Hydrochloride) | | | Acetate) |
| Zoledronic Acid | Zolinza | Zometa (Zoledronic | Zydelig |
| | (Vorinostat) | Acid) | (Idelalisib) |
| Zykadia | Zytiga | | |
| (Ceritinib) | (Abiraterone | | |
| | Acetate) | | |

The disclosure provides the following particular embodiments in connection with treating a disease in a subject.

Embodiment I. A method of treating a subject, the method comprising administering to the subject a therapeutically effective amount of a Compound of the Disclosure or PROTAC Molecule, wherein the subject has cancer or other proliferative disorder, or an inflammatory disease.

Embodiment II. The method Embodiment I, wherein the subject has cancer.

Embodiment III. The method of Embodiment II, wherein the cancer is any one or more of the cancers of Table 4.

Embodiment IV. The method of Embodiment II, wherein the cancer is selected from the group consisting of acute monocytic leukemia, acute myelogenous leukemia, chronic myelogenous leukemia, chronic lymphocytic leukemia mixed lineage leukemia, NUT midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment V. The method of Embodiment II, wherein the cancer is any one or more of the cancers of Table 5, e.g, multiple myeloma.

Embodiment VI. The method of any one of Embodiments I-V further comprising administering a therapeutically effective amount of an optional therapeutic agent useful in the treatment of the disease or condition, e.g., an immune checkpoint inhibitor or other anticancer agent.

Embodiment VII. The method of any one of Embodiments I-VI, wherein the Compound of the Disclosure is a compound of Formula I, or a pharmaceutically acceptable salt or solvate thereof.

Embodiment VIII. The method of any one of Embodiments I-VI, wherein the Compound of the Disclosure is a compound of any one of Formulae II-IV, or a pharmaceutically acceptable salt or solvate thereof.

Embodiment IX. A pharmaceutical composition comprising a Compound of the Disclosure or PROTAC Molecule, and a pharmaceutically acceptable excipient for use in treating cancer or other proliferative disorder, or an inflammatory disease.

Embodiment X. The pharmaceutical composition of Embodiment IX for use in treating cancer.

Embodiment XI. The pharmaceutical composition of Embodiment X, wherein the cancer is any one or more of the cancers of Table 4.

Embodiment XII. The pharmaceutical composition of Embodiment X, wherein the cancer is selected from the group consisting of acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment XIII The pharmaceutical composition of Embodiment X, wherein the cancer is any one or more of the cancers of Table 5.

Embodiment XIV. The pharmaceutical composition of any one of

Embodiments IX-XIII, wherein the Compound of the Disclosure is a compound of Formula I, or a pharmaceutically acceptable salt or solvate thereof.

Embodiment XV. The pharmaceutical composition of any one of

Embodiments IX-XIII, wherein the Compound of the Disclosure is a compound of any one of Formulae II-IV, or a pharmaceutically acceptable salt or solvate thereof.

Embodiment XVI. A Compound of the Disclosure or PROTAC Molecule for use in treatment of cancer or other proliferative disorder, or an inflammatory disease.

Embodiment XVII. The compound of Embodiment XVI for use in treating cancer.

Embodiment XVIII. The compound of Embodiment XVII, wherein the cancer is any one or more of the cancers of Table 4.

Embodiment XIX. The compound of Embodiment XVII, wherein the cancer is selected from the group consisting of acute monocytic leukemia, acute myelogenous leukemia, chronic myelogenous leukemia, chronic lymphocytic leukemia mixed lineage leukemia, NUT midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment XX. The compound of Embodiment XVII, wherein the cancer is any one or more of the cancers of Table 5.

Embodiment XXI. The compound of any one of Embodiments XVI-XX, wherein the Compound of the Disclosure is a compound of Formula I, or a pharmaceutically acceptable salt or solvate thereof.

Embodiment XXII. The compound of any one of Embodiments XVI-XX, wherein the Compound of the Disclosure is a compound of any one of Formulae II-IV, or a pharmaceutically acceptable salt or solvate thereof.

Embodiment XXIII. Use of a Compound of the Disclosure or PROTAC Molecule for the manufacture of a medicament for treatment of cancer or other proliferative disorder, or an inflammatory disease.

Embodiment XXIV. The use of Embodiment XXIII for the treatment of cancer.

Embodiment XXV. The use of Embodiment XXIV, wherein the cancer is any one or more of the cancers of Table 4.

Embodiment XXVI. The use of Embodiment XXIII, wherein the cancer is selected from the group consisting of acute monocytic leukemia, acute myelogenous leukemia, chronic myelogenous leukemia, chronic lymphocytic leukemia mixed lineage leukemia, NUT midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment XXVII. The use of Embodiment XXIV, wherein the cancer is any one or more of the cancers of Table 5.

Embodiment XXVIII. The use of any one of Embodiments XXIII-XXVII, wherein the Compound of the Disclosure is a compound of any one of Formula I, or a pharmaceutically acceptable salt or solvate thereof.

Embodiment XXIX. The use of any one of Embodiments XXIII-XXVII, wherein the Compound of the Disclosure is a compound of any one of Formulae II-IV, or a pharmaceutically acceptable salt or solvate thereof.

Embodiment XXX. A method of inhibiting CRBN ubiquitination within a cell of a subject in need thereof, the method comprising administering to the subject a compound of Formula I, or a pharmaceutically acceptable salt or solvate thereof.

Embodiment XXXI. A method of inhibiting CRBN ubiquitination within a cell of a subject in need thereof, the method comprising administering to the subject a compound of any one of Formulae II-IV, or a pharmaceutically acceptable salt or solvate thereof.

### V. Kits of the Disclosure

In another embodiment, the present disclosure provides kits which comprise a Compound of the Disclosure or PROTAC Molecule (or a composition comprising a Compound of the Disclosure or PROTAC Molecule) packaged in a manner that facilitates their use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure, e.g., the method of any one of Embodiments I-VI. In one embodiment, the compound or composition is packaged in a unit dosage form. The kit further can include a device suitable for administering the composition according to the intended route of administration.

### VI. PROTAC Molecules

Proteolysis-targeting chimera (PROTAC) is a useful technology for targeted protein degradation. A bifunctional PROTAC molecule consists of a ligand (usually a small-molecule inhibitor) of the protein of interest and a covalently linked ligand of an E3 ubiquitin ligase. Upon binding to the protein of interest, the PROTAC can recruit E3 ubiquitin ligase for ubiquitination of the protein of interest, which is subjected to proteasome-mediated degradation. See, e.g., Bondeson and Crews, Annu Rev Pharmacol Toxicol. 57:107-123 (2017); Sun et al., Sig Transduct Target Ther 4:64 (2019) https://doi.org/10.1038/s41392-019-0101-6; Li and Song, J Hematol Oncol 13: 50 (2020) https://doi.org/10.1186/s13045-020-00885-3; Wang et al., Acta Pharmaceutica Sinica B 10:207-238 (2020). Compounds of the Disclosure can be tethered to a moiety of interest, e.g., ligand that binds to a protein, e.g., small molecule inhibitor of a protein, to give a PROTAC molecule.

In one embodiment, PROTAC Molecules are compounds of Formula XXIII:

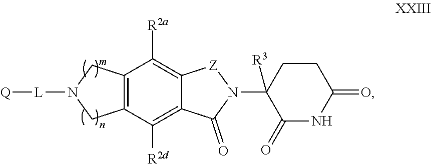

or a pharmaceutically acceptable salt or solvate thereof, wherein:

R^{2a}, R^{2d}, R³, m, n, and Z are as defined in connection with Formula I;

Q is a moiety of interest; and

L is -J¹-J²-J³-J⁴-J⁵-, wherein J¹ is attached to Q;

J¹ is selected from the group consisting of alkylenyl, cycloalkylenyl, and heterocyclenyl; or J¹ is absent;

J² is selected from the group consisting of —C(═O)—, —(CH₂)_{q}—, —CH═CH—, and —C≡C—;

q is 0, 1, 2, or 3;

J³ is selected from the group consisting of alkylenyl, heteroalkylenyl, cycloalkylenyl, heterocyclenyl, phenylenyl, and heteroarylenyl; or J³ is absent;

J⁴ is selected from the group consisting of alkylenyl, cycloalkylenyl, and heterocyclenyl; or J⁴ is absent;

J⁵ is selected from the group consisting of —(CH₂)ᵣ—and —C(═O)—; and

r is 0, 1, 2, or 3.

In another embodiment, PROTAC Molecules are compounds of Formula XXIII, wherein Z is —CH₂—, or a pharmaceutically acceptable salt or solvate thereof.

In another embodiment, PROTAC Molecules are compounds of Formula XXIII, wherein Z is —C(═O)—, or a pharmaceutically acceptable salt or solvate thereof.

In another embodiment, PROTAC Molecules are compounds of Formula XXIII, wherein R^{2a} and R^{2d} are independently selected from the group consisting of hydrogen, fluoro, and chloro, or a pharmaceutically acceptable salt or solvate thereof. In another embodiment, R^{2a} and R^{2d} are hydrogen.

In another embodiment, PROTAC Molecules are compounds of Formula XXIV:

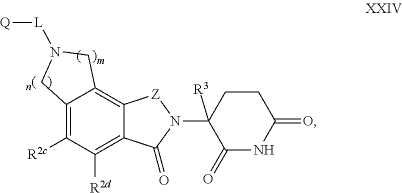

or a pharmaceutically acceptable salt or solvate thereof, wherein L and Q are as defined in connection with Formula XXIII; and R^{2c}, R^{2d}, R³, m, n, and Z are as defined in connection with Formula I.

In another embodiment, PROTAC Molecules are compounds of Formula XXIV, wherein Z is —CH₂—, or a pharmaceutically acceptable salt or solvate thereof.

In another embodiment, PROTAC Molecules are compounds of Formula XXIV, wherein Z is —C(═O)—, or a pharmaceutically acceptable salt or solvate thereof.

In another embodiment, PROTAC Molecules are compounds of Formula XXIV, wherein R^{2c} and R^{2d} are independently selected from the group consisting of hydrogen, fluoro, and chloro, or a pharmaceutically acceptable salt or solvate thereof. In another embodiment, R^{2c} and R^{2d} are hydrogen.

In another embodiment, PROTAC Molecules are compounds of Formula XXV:

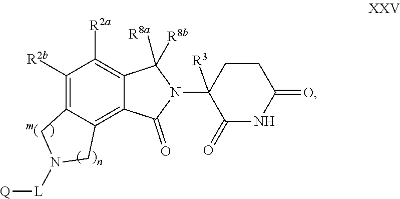

or a pharmaceutically acceptable salt or solvate thereof, wherein L and Q are as defined in connection with Formula XXIII; and R^{2a}, R^{2b}, R³, R^{8a}, R^{8b}, m, n, and Z are as defined in connection with Formula I.

In another embodiment, PROTAC Molecules are compounds of Formula XXV, wherein R^{8a} and R^{8b} are hydrogen, or a pharmaceutically acceptable salt or solvate thereof.

In another embodiment, PROTAC Molecules are compounds of Formula XXV, wherein R^{2a} and R^{2b} are independently selected from the group consisting of hydrogen, fluoro, and chloro, or a pharmaceutically acceptable salt or solvate thereof. In another embodiment, R^{2a} and R^{2b} are hydrogen.

In another embodiment, PROTAC Molecules are compounds of Formula XXVI:

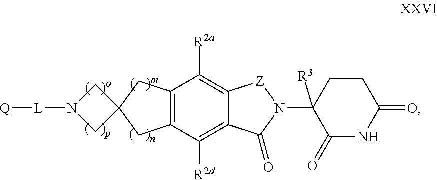

or a pharmaceutically acceptable salt or solvate thereof, wherein L and Q are as defined in connection with Formula XXIII; and R^{2a}, R^{2d}, R³, m, n, and Z are as defined in connection with Formula I.

In another embodiment, PROTAC Molecules are compounds of Formula XXVI, wherein Z is —CH₂—, or a pharmaceutically acceptable salt or solvate thereof.

In another embodiment, PROTAC Molecules are compounds of Formula XXVI, wherein Z is —C(═O)—, or a pharmaceutically acceptable salt or solvate thereof.

In another embodiment, PROTAC Molecules are compounds of Formula XXVI, wherein R^{2a} and R^{2d} are independently selected from the group consisting of hydrogen, fluoro, and chloro, or a pharmaceutically acceptable salt or solvate thereof. In another embodiment, R^{2a} and R^{2d} are hydrogen.

In another embodiment, PROTAC Molecules are compounds of Formula XXVII:

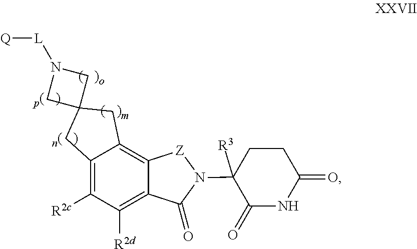

or a pharmaceutically acceptable salt or solvate thereof, wherein L and Q are as defined in connection with Formula XXIII; and R^{2c}, R^{2a}, R^{2d}, R³, m, n, o, p, and Z are as defined in connection with Formula I.

In another embodiment, PROTAC Molecules are compounds of Formula XXVII, wherein Z is —CH₂—, or a pharmaceutically acceptable salt or solvate thereof.

In another embodiment, PROTAC Molecules are compounds of Formula XXVII, wherein Z is —C(═O)—, or a pharmaceutically acceptable salt or solvate thereof.

In another embodiment, PROTAC Molecules are compounds of Formula XXVII, wherein R^{2c} and R^{2d} are independently selected from the group consisting of hydrogen, fluoro, and chloro, or a pharmaceutically acceptable salt or solvate thereof. In another embodiment, R^{2c} and R^{2d} are hydrogen.

In another embodiment, PROTAC Molecules are compounds of Formula XXVIII:

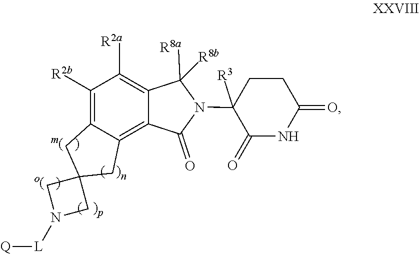

or a pharmaceutically acceptable salt or solvate thereof, wherein L and Q are as defined in connection with Formula XXIII; and R^{2a}, R^{2b}, R³, R^{8a}, R^{8b}, m, n, o, p, and Z are as defined in connection with Formula I.

In another embodiment, PROTAC Molecules are compounds of Formula XXVIII, wherein R^{8a} and R^{8b} are hydrogen, or a pharmaceutically acceptable salt or solvate thereof.

In another embodiment, PROTAC Molecules are compounds of Formula XXVIII, wherein R^{2a} and R^{2b} are independently selected from the group consisting of hydrogen, fluoro, and chloro, or a pharmaceutically acceptable salt or solvate thereof. In another embodiment, R^{2a} and R^{2b} are hydrogen.

In another embodiment, PROTAC Molecules are compounds of Formula XXIX:

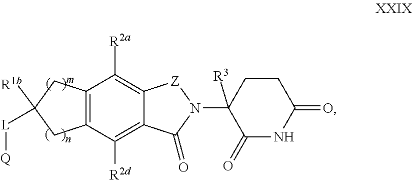

or a pharmaceutically acceptable salt or solvate thereof, wherein:

Q is as defined in connection with Formula XXIII;

R^{1b}, R^{2a}, R^{2d}, R³, m, n, and Z are as defined in connection with Formula I;

L is -J¹-J²-J³-J⁴-J⁵-, wherein J¹ is attached to Q;

J¹, J², j³, J⁴ are as defined in connection with Formula XXIII;

J⁵ is selected from the group consisting of —C≡C—, —(CH₂)ᵣ—, —O—, —N(R¹⁴)—, and —C(═O)—;

r is 0, 1, 2, or 3; and

R¹⁴ is selected from the group consisting of hydrogen and C₁-C₃ alkyl.

In another embodiment, PROTAC Molecules are compounds of Formula XXIX, wherein Z is —CH₂—, or a pharmaceutically acceptable salt or solvate thereof.

In another embodiment, PROTAC Molecules are compounds of Formula XXIX, wherein Z is —C(═O)—, or a pharmaceutically acceptable salt or solvate thereof.

In another embodiment, PROTAC Molecules are compounds of Formula XXIX, wherein R^{2a} and R^{2d} are independently selected from the group consisting of hydrogen, fluoro, and chloro, or a pharmaceutically acceptable salt or solvate thereof. In another embodiment, R^{2a} and R^{2d} are hydrogen.

In another embodiment, PROTAC Molecules are compounds of Formula XXX:

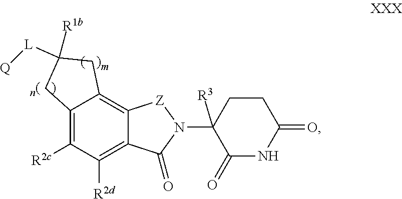

or a pharmaceutically acceptable salt or solvate thereof, wherein:

Q is as defined in connection with Formula XXIII;

R^{1b}, R^{2c}, R^{2d}, R³, m, n, and Z are as defined in connection with Formula I;

L is -J¹-J²-J³-J⁴-J⁵-, wherein J¹ is attached to Q;

J¹, J², J³, J⁴ are as defined in connection with Formula XXIII; and

J⁵ is as defined in connection with Formula XXIX.

In another embodiment, PROTAC Molecules are compounds of Formula XXX, wherein Z is —CH₂—, or a pharmaceutically acceptable salt or solvate thereof.

In another embodiment, PROTAC Molecules are compounds of Formula XXX, wherein Z is —C(═O)—, or a pharmaceutically acceptable salt or solvate thereof.

In another embodiment, PROTAC Molecules are compounds of Formula XXX, wherein R^{2c} and R^{2d} are independently selected from the group consisting of hydrogen, fluoro, and chloro, or a pharmaceutically acceptable salt or solvate thereof. In another embodiment, R^{2c} and R^{2d} are hydrogen.

In another embodiment, PROTAC Molecules are compounds of Formula XXXI:

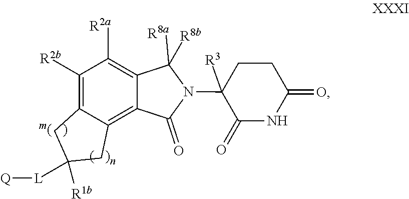

or a pharmaceutically acceptable salt or solvate thereof, wherein:

Q is as defined in connection with Formula XXIII;

R^{1b}, R^{2a}, R^{2b}, R³, R^{8a}, R^{8b}, m, n, and Zare as defined in connection with Formula I;

L is -J¹-J²-J³-J⁴-J⁵-, wherein J¹ is attached to Q;

J¹, J², J³, J⁴ are as defined in connection with Formula XXIII; and

J⁵ is as defined in connection with Formula XXIX.

In another embodiment, PROTAC Molecules are compounds of Formula XXXI, wherein R^{8a} and R^{8b} are hydrogen, or a pharmaceutically acceptable salt or solvate thereof.

In another embodiment, PROTAC Molecules are compounds of Formula XXXI, wherein R^{2a} and R^{2b} are independently selected from the group consisting of hydrogen, fluoro, and chloro, or a pharmaceutically acceptable salt or solvate thereof. In another embodiment, R^{2a} and R^{2b} are hydrogen.

In another embodiment, PROTAC Molecules are compounds of Formula XXXII:

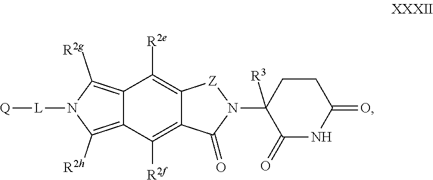

or a pharmaceutically acceptable salt or solvate thereof, wherein:

L and Q are as defined in connection with Formula XXIII;

R^{2e}, R^{2f}, R^{2g}, and R^{2h} are as defined in connection with Formula XVIII; and

R³ and Z as defined in connection with Formula I.

In another embodiment, PROTAC Molecules are compounds of Formula XXXII, wherein Z is —CH₂—, or a pharmaceutically acceptable salt or solvate thereof.

In another embodiment, PROTAC Molecules are compounds of Formula XXXII, wherein Z is —C(═O)—, or a pharmaceutically acceptable salt or solvate thereof.

In another embodiment, PROTAC Molecules are compounds of Formula XXXII, wherein R^{2e} and R^{2f} are independently selected from the group consisting of hydrogen and halo, or a pharmaceutically acceptable salt or solvate thereof. In another embodiment, R^{2e} and R^{2f} are hydrogen.

In another embodiment, PROTAC Molecules are compounds of Formula XXXII, wherein R^{2g} and R^{2h} are independently selected from the group consisting of hydrogen, halo, and C₁-C₃ alkyl, or a pharmaceutically acceptable salt or solvate thereof. In another embodiment, R^{2g} and R^{2h} are hydrogen.

In another embodiment, PROTAC Molecules are compounds of Formula XXXIII:

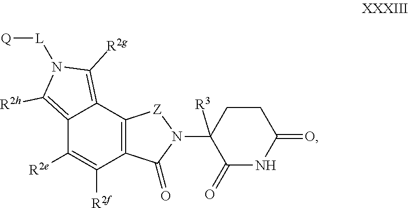

or a pharmaceutically acceptable salt or solvate thereof, wherein:

L and Q are as defined in connection with Formula XXIII;

R^{2e}, R^{2f}, R^{2g}, and R^{2h} are as defined in connection with Formula XVIII; and

R³ and Z as defined in connection with Formula I.

In another embodiment, PROTAC Molecules are compounds of Formula XXXIII, wherein Z is —CH₂—, or a pharmaceutically acceptable salt or solvate thereof.

In another embodiment, PROTAC Molecules are compounds of Formula XXXIII, wherein Z is —C(═O)—, or a pharmaceutically acceptable salt or solvate thereof.

In another embodiment, PROTAC Molecules are compounds of Formula XXXIII, wherein R^{2c} and R^{2f} are independently selected from the group consisting of hydrogen and halo, or a pharmaceutically acceptable salt or solvate thereof. In another embodiment, R^{2e} and R^{2f} are hydrogen.

In another embodiment, PROTAC Molecules are compounds of Formula XXXIII, wherein R^{2g} and R^{2h} are independently selected from the group consisting of hydrogen, halo, and C₁-C₃ alkyl, or a pharmaceutically acceptable salt or solvate thereof. In another embodiment, R^{2g} and R^{2h} are hydrogen

In another embodiment, PROTAC Molecules are compounds of Formula XXXIV:

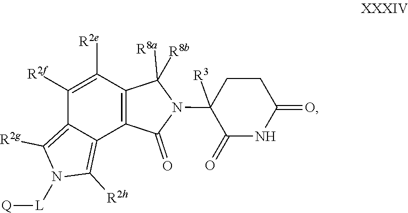

or a pharmaceutically acceptable salt or solvate thereof, wherein:

L and Q are as defined in connection with Formula XXIII;

R^{2e}, R^{2f}, R^{2g}, and R^{2h} are as defined in connection with Formula XVIII; and

R³, R^{8a}, R^{8b}, and Z as defined in connection with Formula I.

In another embodiment, PROTAC Molecules are compounds of Formula XXXIV, wherein R^{8a} and R^{8b} are hydrogen, or a pharmaceutically acceptable salt or solvate thereof.

In another embodiment, PROTAC Molecules are compounds of Formula XXXIV, wherein R^{2e} and R^{2f} are independently selected from the group consisting of hydrogen and halo, or a pharmaceutically acceptable salt or solvate thereof. In another embodiment, R^{2e} and R^{2f} are hydrogen.

In another embodiment, PROTAC Molecules are compounds of Formula XXXIV, wherein R^{2g} and R^{2h} are independently selected from the group consisting of hydrogen, halo, and C₁-C₃ alkyl, or a pharmaceutically acceptable salt or solvate thereof. In another embodiment, R^{2g} and R^{2h} are hydrogen.

In another embodiment, PROTAC Molecules are compounds of any one of Formulae XXIII-XXVIII or XXXII-XXXIV, or a pharmaceutically acceptable salt or solvate thereof, wherein L is any one or more of the -J¹-, -J¹-J²-, -J¹-J²-J³-, or J¹-J²-J³-J⁴-groups listed in Table 9.

In another embodiment, PROTAC Molecules are compounds of any one of Formulae XXIX-XXXI, or a pharmaceutically acceptable salt or solvate thereof, wherein L is any one or more of the-J¹-, -J¹-J²-, -J¹-J²-J³-, or J¹-J²-J³-J⁴- or , -J¹-J²-J³-J⁴-J⁵- groups listed in Table 9.

TABLE 9

| No. | J¹ | J² | J³ | J⁴ | J⁵ |
|---|---|---|---|---|---|
| 1 | alkylenyl | — | — | — | — |
| 2 | cycloalkylenyl | — | — | — | — |
| 3 | heterocyclenyl | — | — | — | — |
| 4 | — | —C(═O)— | — | — | — |
| 5 | alkylenyl | —C(═O)— | — | — | — |
| 6 | cycloalkylenyl | —C(═O)— | — | — | — |
| 7 | heterocyclenyl | —C(═O)— | — | — | — |
| 8 | — | —C(═O)NH— | — | — | — |
| 9 | alkylenyl | —C(═O)NH— | — | — | — |
| 10 | cycloalkylenyl | —C(═O)NH— | — | — | — |
| 11 | heterocyclenyl | —C(═O)NH— | — | — | — |
| 12 | — | —C≡C— | — | — | — |
| 13 | alkylenyl | —C≡C— | — | — | — |
| 14 | cycloalkylenyl | —C≡C— | — | — | — |
| 15 | heterocyclenyl | —C≡C— | — | — | — |
| 16 | alkylenyl | — | heterocyclenyl | — | — |
| 17 | cycloalkylenyl | — | heterocyclenyl | — | — |
| 18 | heterocyclenyl | — | heterocyclenyl | — | — |
| 19 | — | —C(═O)— | heterocyclenyl | — | — |
| 20 | alkylenyl | —C(═O)— | heterocyclenyl | — | — |
| 21 | cycloalkylenyl | —C(═O)— | heterocyclenyl | — | — |
| 22 | heterocyclenyl | —C(═O)— | heterocyclenyl | — | — |
| 23 | — | —C(═O)NH— | heterocyclenyl | — | — |
| 24 | alkylenyl | —C(═O)NH— | heterocyclenyl | — | — |
| 25 | cycloalkylenyl | —C(═O)NH— | heterocyclenyl | — | — |
| 26 | heterocyclenyl | —C(═O)NH— | heterocyclenyl | — | — |
| 27 | — | —C≡C— | heterocyclenyl | — | — |
| 28 | alkylenyl | —C≡C— | heterocyclenyl | — | — |
| 29 | cycloalkylenyl | —C≡C— | heterocyclenyl | — | — |
| 30 | heterocyclenyl | —C≡C— | heterocyclenyl | — | — |
| 31 | cycloalkylenyl | — | alkylenyl | heterocyclenyl | — |
| 32 | heterocyclenyl | — | alkylenyl | heterocyclenyl | — |
| 33 | — | —C(═O)— | alkylenyl | heterocyclenyl | — |
| 34 | alkylenyl | —C(═O)— | alkylenyl | heterocyclenyl | — |
| 35 | cycloalkylenyl | —C(═O)— | alkylenyl | heterocyclenyl | — |
| 36 | heterocyclenyl | —C(═O)— | alkylenyl | heterocyclenyl | — |
| 37 | — | —C(═O)NH— | alkylenyl | heterocyclenyl | — |
| 38 | alkylenyl | —C(═O)NH— | alkylenyl | heterocyclenyl | — |
| 39 | cycloalkylenyl | —C(═O)NH— | alkylenyl | heterocyclenyl | — |
| 40 | heterocyclenyl | —C(═O)NH— | alkylenyl | heterocyclenyl | — |
| 41 | — | —C≡C— | alkylenyl | heterocyclenyl | — |
| 42 | alkylenyl | —C≡C— | alkylenyl | heterocyclenyl | — |
| 43 | cycloalkylenyl | —C≡C— | alkylenyl | heterocyclenyl | — |
| 44 | heterocyclenyl | —C≡C— | alkylenyl | heterocyclenyl | — |
| 45 | alkylenyl | — | cycloalkylenyl | heterocyclenyl | — |
| 46 | cycloalkylenyl | — | cycloalkylenyl | heterocyclenyl | — |
| 47 | heterocyclenyl | — | cycloalkylenyl | heterocyclenyl | — |
| 48 | — | —C(═O)— | cycloalkylenyl | heterocyclenyl | — |
| 49 | alkylenyl | —C(═O)— | cycloalkylenyl | heterocyclenyl | — |
| 50 | cycloalkylenyl | —C(═O)— | cycloalkylenyl | heterocyclenyl | — |
| 51 | heterocyclenyl | —C(═O)— | cycloalkylenyl | heterocyclenyl | — |
| 52 | — | —C(═O)NH— | cycloalkylenyl | heterocyclenyl | — |
| 53 | alkylenyl | —C(═O)NH— | cycloalkylenyl | heterocyclenyl | — |
| 54 | cycloalkylenyl | —C(═O)NH— | cycloalkylenyl | heterocyclenyl | — |
| 55 | heterocyclenyl | —C(═O)NH— | cycloalkylenyl | heterocyclenyl | — |
| 56 | — | —C≡C— | cycloalkylenyl | heterocyclenyl | — |
| 57 | alkylenyl | —C≡C— | cycloalkylenyl | heterocyclenyl | — |
| 58 | cycloalkylenyl | —C≡C— | cycloalkylenyl | heterocyclenyl | — |
| 59 | heterocyclenyl | —C≡C— | cycloalkylenyl | heterocyclenyl | — |
| 60 | alkylenyl | — | phenylenyl | heterocyclenyl | — |
| 61 | cycloalkylenyl | — | phenylenyl | heterocyclenyl | — |
| 62 | heterocyclenyl | — | phenylenyl | heterocyclenyl | — |
| 63 | — | —C(═O)— | phenylenyl | heterocyclenyl | — |
| 64 | alkylenyl | —C(═O)— | phenylenyl | heterocyclenyl | — |
| 65 | cycloalkylenyl | —C(═O)— | phenylenyl | heterocyclenyl | — |
| 66 | heterocyclenyl | —C(═O)— | phenylenyl | heterocyclenyl | — |
| 67 | — | —C(═O)NH— | phenylenyl | heterocyclenyl | — |
| 68 | alkylenyl | —C(═O)NH— | phenylenyl | heterocyclenyl | — |
| 69 | cycloalkylenyl | —C(═O)NH— | phenylenyl | heterocyclenyl | — |
| 70 | heterocyclenyl | —C(═O)NH— | phenylenyl | heterocyclenyl | — |
| 71 | — | —C≡C— | phenylenyl | heterocyclenyl | — |
| 72 | alkylenyl | —C≡C— | phenylenyl | heterocyclenyl | — |
| 73 | cycloalkylenyl | —C≡C— | phenylenyl | heterocyclenyl | — |
| 74 | heterocyclenyl | —C≡C— | phenylenyl | heterocyclenyl | — |
| 75 | cycloalkylenyl | — | alkylenyl | — | —C≡C— |
| 76 | heterocyclenyl | — | alkylenyl | — | —C≡C— |
| 77 | — | —C(═O)— | alkylenyl | — | —C≡C— |
| 78 | alkylenyl | —C(═O)— | alkylenyl | — | —C≡C— |
| 79 | cycloalkylenyl | —C(═O)— | alkylenyl | — | —C≡C— |
| 80 | heterocyclenyl | —C(═O)— | alkylenyl | — | —C≡C— |
| 81 | — | —C(═O)NH— | alkylenyl | — | —C≡C— |
| 82 | alkylenyl | —C(═O)NH— | alkylenyl | — | —C≡C— |
| 83 | cycloalkylenyl | —C(═O)NH— | alkylenyl | — | —C≡C— |
| 84 | heterocyclenyl | —C(═O)NH— | alkylenyl | — | —C≡C— |
| 85 | — | —C≡C— | alkylenyl | — | —C≡C— |
| 86 | alkylenyl | —C≡C— | alkylenyl | — | —C≡C— |
| 87 | cycloalkylenyl | —C≡C— | alkylenyl | — | —C≡C— |
| 88 | heterocyclenyl | —C≡C— | alkylenyl | — | —C≡C— |
| 89 | — | — | heteroalkylenyl | — | —C≡C— |
| 90 | alkylenyl | — | heteroalkylenyl | — | —C≡C— |
| 91 | cycloalkylenyl | — | heteroalkylenyl | — | —C≡C— |
| 92 | heterocyclenyl | — | heteroalkylenyl | — | —C≡C— |
| 93 | — | —C(═O)— | heteroalkylenyl | — | —C≡C— |
| 94 | alkylenyl | —C(═O)— | heteroalkylenyl | — | —C≡C— |
| 95 | cycloalkylenyl | —C(═O)— | heteroalkylenyl | — | —C≡C— |
| 96 | heterocyclenyl | —C(═O)— | heteroalkylenyl | — | —C≡C— |
| 97 | — | —C(═O)NH— | heteroalkylenyl | — | —C≡C— |
| 98 | alkylenyl | —C(═O)NH— | heteroalkylenyl | — | —C≡C— |
| 99 | cycloalkylenyl | —C(═O)NH— | heteroalkylenyl | — | —C≡C— |
| 100 | heterocyclenyl | —C(═O)NH— | heteroalkylenyl | — | —C≡C— |
| 101 | — | —C≡C— | heteroalkylenyl | — | —C≡C— |
| 102 | alkylenyl | —C≡C— | heteroalkylenyl | — | —C≡C— |
| 103 | cycloalkylenyl | —C≡C— | heteroalkylenyl | — | —C≡C— |
| 104 | heterocyclenyl | —C≡C— | heteroalkylenyl | — | —C≡C— |
| 105 | alkylenyl | — | heterocyclenyl | — | —C≡C— |
| 106 | cycloalkylenyl | — | heterocyclenyl | — | —C≡C— |
| 107 | heterocyclenyl | — | heterocyclenyl | — | —C≡C— |
| 108 | — | —C(═O)— | heterocyclenyl | — | —C≡C— |
| 109 | alkylenyl | —C(═O)— | heterocyclenyl | — | —C≡C— |
| 110 | cycloalkylenyl | —C(═O)— | heterocyclenyl | — | —C≡C— |
| 111 | heterocyclenyl | —C(═O)— | heterocyclenyl | — | —C≡C— |
| 112 | — | —C(═O)NH— | heterocyclenyl | — | —C≡C— |
| 113 | alkylenyl | —C(═O)NH— | heterocyclenyl | — | —C≡C— |
| 114 | cycloalkylenyl | —C(═O)NH— | heterocyclenyl | — | —C≡C— |
| 115 | heterocyclenyl | —C(═O)NH— | heterocyclenyl | — | —C≡C— |
| 116 | — | —C≡C— | heterocyclenyl | — | —C≡C— |
| 117 | alkylenyl | —C≡C— | heterocyclenyl | — | —C≡C— |
| 118 | cycloalkylenyl | —C≡C— | heterocyclenyl | — | —C≡C— |
| 119 | heterocyclenyl | —C≡C— | heterocyclenyl | — | —C≡C— |
| 120 | alkylenyl | — | alkylenyl | heterocyclenyl | —C≡C— |
| 121 | cycloalkylenyl | — | alkylenyl | heterocyclenyl | —C≡C— |
| 122 | heterocyclenyl | — | alkylenyl | heterocyclenyl | —C≡C— |
| 123 | — | —C(═O)— | alkylenyl | heterocyclenyl | —C≡C— |
| 124 | alkylenyl | —C(═O)— | alkylenyl | heterocyclenyl | —C≡C— |
| 125 | cycloalkylenyl | —C(═O)— | alkylenyl | heterocyclenyl | —C≡C— |
| 126 | heterocyclenyl | —C(═O)— | alkylenyl | heterocyclenyl | —C≡C— |
| 127 | — | —C(═O)NH— | alkylenyl | heterocyclenyl | —C≡C— |
| 128 | alkylenyl | —C(═O)NH— | alkylenyl | heterocyclenyl | —C≡C— |
| 129 | cycloalkylenyl | —C(═O)NH— | alkylenyl | heterocyclenyl | —C≡C— |
| 130 | heterocyclenyl | —C(═O)NH— | alkylenyl | heterocyclenyl | —C≡C— |
| 131 | — | —C≡C— | alkylenyl | heterocyclenyl | —C≡C— |
| 132 | alkylenyl | —C≡C— | alkylenyl | heterocyclenyl | —C≡C— |
| 133 | cycloalkylenyl | —C≡C— | alkylenyl | heterocyclenyl | —C≡C— |
| 134 | heterocyclenyl | —C≡C— | alkylenyl | heterocyclenyl | —C≡C— |
| 135 | alkylenyl | — | heterocyclenyl | alkylenyl | —C≡C— |
| 136 | cycloalkylenyl | — | heterocyclenyl | alkylenyl | —C≡C— |
| 137 | heterocyclenyl | — | heterocyclenyl | alkylenyl | —C≡C— |
| 138 | — | —C(═O)— | heterocyclenyl | alkylenyl | —C≡C— |
| 139 | alkylenyl | —C(═O)— | heterocyclenyl | alkylenyl | —C≡C— |
| 140 | cycloalkylenyl | —C(═O)— | heterocyclenyl | alkylenyl | —C≡C— |
| 141 | heterocyclenyl | —C(═O)— | heterocyclenyl | alkylenyl | —C≡C— |
| 142 | — | —C(═O)NH— | heterocyclenyl | alkylenyl | —C≡C— |
| 143 | alkylenyl | —C(═O)NH— | heterocyclenyl | alkylenyl | —C≡C— |
| 144 | cycloalkylenyl | —C(═O)NH— | heterocyclenyl | alkylenyl | —C≡C— |
| 145 | heterocyclenyl | —C(═O)NH— | heterocyclenyl | alkylenyl | —C≡C— |
| 146 | — | —C≡C— | heterocyclenyl | alkylenyl | —C≡C— |
| 147 | alkylenyl | —C≡C— | heterocyclenyl | alkylenyl | —C≡C— |
| 148 | cycloalkylenyl | —C≡C— | heterocyclenyl | alkylenyl | —C≡C— |
| 149 | heterocyclenyl | —C≡C— | heterocyclenyl | alkylenyl | —C≡C— |
| 150 | alkylenyl | — | — | cycloalkylenyl | —C≡C— |
| 151 | cycloalkylenyl | — | — | cycloalkylenyl | —C≡C— |
| 152 | heterocyclenyl | — | — | cycloalkylenyl | —C≡C— |
| 153 | — | —C(═O)— | — | cycloalkylenyl | —C≡C— |
| 154 | alkylenyl | —C(═O)— | — | cycloalkylenyl | —C≡C— |
| 155 | cycloalkylenyl | —C(═O)— | — | cycloalkylenyl | —C≡C— |
| 156 | heterocyclenyl | —C(═O)— | — | cycloalkylenyl | —C≡C— |
| 157 | — | —C(═O)NH— | — | cycloalkylenyl | —C≡C— |
| 158 | alkylenyl | —C(═O)NH— | — | cycloalkylenyl | —C≡C— |
| 159 | cycloalkylenyl | —C(═O)NH— | — | cycloalkylenyl | —C≡C— |
| 160 | heterocyclenyl | —C(═O)NH— | — | cycloalkylenyl | —C≡C— |
| 161 | — | —C≡C— | — | cycloalkylenyl | —C≡C— |
| 162 | alkylenyl | —C≡C— | — | cycloalkylenyl | —C≡C— |
| 163 | cycloalkylenyl | —C≡C— | — | cycloalkylenyl | —C≡C— |
| 164 | heterocyclenyl | —C≡C— | — | cycloalkylenyl | —C≡C— |
| 165 | alkylenyl | — | alkylenyl | cycloalkylenyl | —C≡C— |
| 166 | cycloalkylenyl | — | alkylenyl | cycloalkylenyl | —C≡C— |
| 167 | heterocyclenyl | — | alkylenyl | cycloalkylenyl | —C≡C— |
| 168 | — | —C(═O)— | alkylenyl | cycloalkylenyl | —C≡C— |
| 169 | alkylenyl | —C(═O)— | alkylenyl | cycloalkylenyl | —C≡C— |
| 170 | cycloalkylenyl | —C(═O)— | alkylenyl | cycloalkylenyl | —C≡C— |
| 171 | heterocyclenyl | —C(═O)— | alkylenyl | cycloalkylenyl | —C≡C— |
| 172 | — | —C(═O)NH— | alkylenyl | cycloalkylenyl | —C≡C— |
| 173 | alkylenyl | —C(═O)NH— | alkylenyl | cycloalkylenyl | —C≡C— |
| 174 | cycloalkylenyl | —C(═O)NH— | alkylenyl | cycloalkylenyl | —C≡C— |
| 175 | heterocyclenyl | —C(═O)NH— | alkylenyl | cycloalkylenyl | —C≡C— |
| 176 | — | —C≡C— | alkylenyl | cycloalkylenyl | —C≡C— |
| 177 | alkylenyl | —C≡C— | alkylenyl | cycloalkylenyl | —C≡C— |
| 178 | cycloalkylenyl | —C≡C— | alkylenyl | cycloalkylenyl | —C≡C— |
| 179 | heterocyclenyl | —C≡C— | alkylenyl | cycloalkylenyl | —C≡C— |
| 180 | alkylenyl | — | heterocyclenyl | cycloalkylenyl | —C≡C— |
| 181 | cycloalkylenyl | — | heterocyclenyl | cycloalkylenyl | —C≡C— |
| 182 | heterocyclenyl | — | heterocyclenyl | cycloalkylenyl | —C≡C— |
| 183 | — | —C(═O)— | heterocyclenyl | cycloalkylenyl | —C≡C— |
| 184 | alkylenyl | —C(═O)— | heterocyclenyl | cycloalkylenyl | —C≡C— |
| 185 | cycloalkylenyl | —C(═O)— | heterocyclenyl | cycloalkylenyl | —C≡C— |
| 186 | heterocyclenyl | —C(═O)— | heterocyclenyl | cycloalkylenyl | —C≡C— |
| 187 | — | —C(═O)NH— | heterocyclenyl | cycloalkylenyl | —C≡C— |
| 188 | alkylenyl | —C(═O)NH— | heterocyclenyl | cycloalkylenyl | —C≡C— |
| 189 | cycloalkylenyl | —C(═O)NH— | heterocyclenyl | cycloalkylenyl | —C≡C— |
| 190 | heterocyclenyl | —C(═O)NH— | heterocyclenyl | cycloalkylenyl | —C≡C— |
| 191 | — | —C≡C— | heterocyclenyl | cycloalkylenyl | —C≡C— |
| 192 | alkylenyl | —C≡C— | heterocyclenyl | cycloalkylenyl | —C≡C— |
| 193 | cycloalkylenyl | —C≡C— | heterocyclenyl | cycloalkylenyl | —C≡C— |
| 194 | heterocyclenyl | —C≡C— | heterocyclenyl | cycloalkylenyl | —C≡C— |
| 195 | alkylenyl | — | — | — | —O— |
| 196 | cycloalkylenyl | — | — | — | —O— |
| 197 | heterocyclenyl | — | — | — | —O— |
| 198 | cycloalkylenyl | — | alkylenyl | — | —O— |
| 199 | heterocyclenyl | — | alkylenyl | — | —O— |
| 200 | — | —C(═O)— | alkylenyl | — | —O— |
| 201 | alkylenyl | —C(═O)— | alkylenyl | — | —O— |
| 202 | cycloalkylenyl | —C(═O)— | alkylenyl | — | —O— |
| 203 | heterocyclenyl | —C(═O)— | alkylenyl | — | —O— |
| 204 | — | —C(═O)NH— | alkylenyl | — | —O— |
| 205 | alkylenyl | —C(═O)NH— | alkylenyl | — | —O— |
| 206 | cycloalkylenyl | —C(═O)NH— | alkylenyl | — | —O— |
| 207 | heterocyclenyl | —C(═O)NH— | alkylenyl | — | —O— |
| 208 | — | —C≡C— | alkylenyl | — | —O— |
| 209 | alkylenyl | —C≡C— | alkylenyl | — | —O— |
| 210 | cycloalkylenyl | —C≡C— | alkylenyl | — | —O— |
| 211 | heterocyclenyl | —C≡C— | alkylenyl | — | —O— |
| 212 | alkylenyl | — | heterocyclenyl | — | —O— |
| 213 | cycloalkylenyl | — | heterocyclenyl | — | —O— |
| 214 | heterocyclenyl | — | heterocyclenyl | — | —O— |
| 215 | — | —C(═O)— | heterocyclenyl | — | —O— |
| 216 | alkylenyl | —C(═O)— | heterocyclenyl | — | —O— |
| 217 | cycloalkylenyl | —C(═O)— | heterocyclenyl | — | —O— |
| 218 | heterocyclenyl | —C(═O)— | heterocyclenyl | — | —O— |
| 219 | — | —C(═O)NH— | heterocyclenyl | — | —O— |
| 220 | alkylenyl | —C(═O)NH— | heterocyclenyl | — | —O— |
| 221 | cycloalkylenyl | —C(═O)NH— | heterocyclenyl | — | —O— |
| 222 | heterocyclenyl | —C(═O)NH— | heterocyclenyl | — | —O— |
| 223 | — | —C≡C— | heterocyclenyl | — | —O— |
| 224 | alkylenyl | —C≡C— | heterocyclenyl | — | —O— |
| 225 | cycloalkylenyl | —C≡C— | heterocyclenyl | — | —O— |
| 226 | heterocyclenyl | —C≡C— | heterocyclenyl | — | —O— |
| 227 | alkylenyl | — | alkylenyl | heterocyclenyl | —O— |
| 228 | cycloalkylenyl | — | alkylenyl | heterocyclenyl | —O— |
| 229 | heterocyclenyl | — | alkylenyl | heterocyclenyl | —O— |
| 230 | — | —C(═O)— | alkylenyl | heterocyclenyl | —O— |
| 231 | alkylenyl | —C(═O)— | alkylenyl | heterocyclenyl | —O— |
| 232 | cycloalkylenyl | —C(═O)— | alkylenyl | heterocyclenyl | —O— |
| 233 | heterocyclenyl | —C(═O)— | alkylenyl | heterocyclenyl | —O— |
| 234 | — | —C(═O)NH— | alkylenyl | heterocyclenyl | —O— |
| 235 | alkylenyl | —C(═O)NH— | alkylenyl | heterocyclenyl | —O— |
| 236 | cycloalkylenyl | —C(═O)NH— | alkylenyl | heterocyclenyl | —O— |
| 237 | heterocyclenyl | —C(═O)NH— | alkylenyl | heterocyclenyl | —O— |
| 238 | — | —C≡C— | alkylenyl | heterocyclenyl | —O— |
| 239 | alkylenyl | —C≡C— | alkylenyl | heterocyclenyl | —O— |
| 240 | cycloalkylenyl | —C≡C— | alkylenyl | heterocyclenyl | —O— |
| 241 | heterocyclenyl | —C≡C— | alkylenyl | heterocyclenyl | —O— |
| 242 | alkylenyl | — | heterocyclenyl | alkylenyl | —O— |
| 243 | cycloalkylenyl | — | heterocyclenyl | alkylenyl | —O— |
| 244 | heterocyclenyl | — | heterocyclenyl | alkylenyl | —O— |
| 245 | — | —C(═O)— | heterocyclenyl | alkylenyl | —O— |
| 246 | alkylenyl | —C(═O)— | heterocyclenyl | alkylenyl | —O— |
| 247 | cycloalkylenyl | —C(═O)— | heterocyclenyl | alkylenyl | —O— |
| 248 | heterocyclenyl | —C(═O)— | heterocyclenyl | alkylenyl | —O— |
| 249 | — | —C(═O)NH— | heterocyclenyl | alkylenyl | —O— |
| 250 | alkylenyl | —C(═O)NH— | heterocyclenyl | alkylenyl | —O— |
| 251 | cycloalkylenyl | —C(═O)NH— | heterocyclenyl | alkylenyl | —O— |
| 252 | heterocyclenyl | —C(═O)NH— | heterocyclenyl | alkylenyl | —O— |
| 253 | — | —C≡C— | heterocyclenyl | alkylenyl | —O— |
| 254 | alkylenyl | —C≡C— | heterocyclenyl | alkylenyl | —O— |
| 255 | cycloalkylenyl | —C≡C— | heterocyclenyl | alkylenyl | —O— |
| 256 | heterocyclenyl | —C≡C— | heterocyclenyl | alkylenyl | —O— |
| 257 | alkylenyl | — | — | cycloalkylenyl | —O— |
| 258 | cycloalkylenyl | — | — | cycloalkylenyl | —O— |
| 259 | heterocyclenyl | — | — | cycloalkylenyl | —O— |
| 260 | — | —C(═O)— | — | cycloalkylenyl | —O— |
| 261 | alkylenyl | —C(═O)— | — | cycloalkylenyl | —O— |
| 262 | cycloalkylenyl | —C(═O)— | — | cycloalkylenyl | —O— |
| 263 | heterocyclenyl | —C(═O)— | — | cycloalkylenyl | —O— |
| 264 | — | —C(═O)NH— | — | cycloalkylenyl | —O— |
| 265 | alkylenyl | —C(═O)NH— | — | cycloalkylenyl | —O— |
| 266 | cycloalkylenyl | —C(═O)NH— | — | cycloalkylenyl | —O— |
| 267 | heterocyclenyl | —C(═O)NH— | — | cycloalkylenyl | —O— |
| 268 | — | —C≡C— | — | cycloalkylenyl | —O— |
| 269 | alkylenyl | —C≡C— | — | cycloalkylenyl | —O— |
| 270 | cycloalkylenyl | —C≡C— | — | cycloalkylenyl | —O— |
| 271 | heterocyclenyl | —C≡C— | — | cycloalkylenyl | —O— |
| 272 | alkylenyl | — | alkylenyl | cycloalkylenyl | —O— |
| 273 | cycloalkylenyl | — | alkylenyl | cycloalkylenyl | —O— |
| 274 | heterocyclenyl | — | alkylenyl | cycloalkylenyl | —O— |
| 275 | — | —C(═O)— | alkylenyl | cycloalkylenyl | —O— |
| 276 | alkylenyl | —C(═O)— | alkylenyl | cycloalkylenyl | —O— |
| 277 | cycloalkylenyl | —C(═O)— | alkylenyl | cycloalkylenyl | —O— |
| 278 | heterocyclenyl | —C(═O)— | alkylenyl | cycloalkylenyl | —O— |
| 279 | — | —C(═O)NH— | alkylenyl | cycloalkylenyl | —O— |
| 280 | alkylenyl | —C(═O)NH— | alkylenyl | cycloalkylenyl | —O— |
| 281 | cycloalkylenyl | —C(═O)NH— | alkylenyl | cycloalkylenyl | —O— |
| 282 | heterocyclenyl | —C(═O)NH— | alkylenyl | cycloalkylenyl | —O— |
| 283 | — | —C≡C— | alkylenyl | cycloalkylenyl | —O— |
| 284 | alkylenyl | —C≡C— | alkylenyl | cycloalkylenyl | —O— |
| 285 | cycloalkylenyl | —C≡C— | alkylenyl | cycloalkylenyl | —O— |
| 286 | heterocyclenyl | —C≡C— | alkylenyl | cycloalkylenyl | —O— |
| 287 | alkylenyl | — | heterocyclenyl | cycloalkylenyl | —O— |
| 288 | cycloalkylenyl | — | heterocyclenyl | cycloalkylenyl | —O— |
| 289 | heterocyclenyl | — | heterocyclenyl | cycloalkylenyl | —O— |
| 290 | — | —C(═O)— | heterocyclenyl | cycloalkylenyl | —O— |
| 291 | alkylenyl | —C(═O)— | heterocyclenyl | cycloalkylenyl | —O— |
| 292 | cycloalkylenyl | —C(═O)— | heterocyclenyl | cycloalkylenyl | —O— |
| 293 | heterocyclenyl | —C(═O)— | heterocyclenyl | cycloalkylenyl | —O— |
| 294 | — | —C(═O)NH— | heterocyclenyl | cycloalkylenyl | —O— |
| 295 | alkylenyl | —C(═O)NH— | heterocyclenyl | cycloalkylenyl | —O— |
| 296 | cycloalkylenyl | —C(═O)NH— | heterocyclenyl | cycloalkylenyl | —O— |
| 297 | heterocyclenyl | —C(═O)NH— | heterocyclenyl | cycloalkylenyl | —O— |
| 298 | — | —C≡C— | heterocyclenyl | cycloalkylenyl | —O— |
| 299 | alkylenyl | —C≡C— | heterocyclenyl | cycloalkylenyl | —O— |
| 300 | cycloalkylenyl | —C≡C— | heterocyclenyl | cycloalkylenyl | —O— |
| 301 | heterocyclenyl | —C≡C— | heterocyclenyl | cycloalkylenyl | —O— |
| 302 | alkylenyl | — | — | — | —NH— |
| 303 | cycloalkylenyl | — | — | — | —NH— |
| 304 | heterocyclenyl | — | — | — | —NH— |
| 305 | cycloalkylenyl | — | alkylenyl | — | —NH— |
| 306 | heterocyclenyl | — | alkylenyl | — | —NH— |
| 307 | — | —C(═O)— | alkylenyl | — | —NH— |
| 308 | alkylenyl | —C(═O)— | alkylenyl | — | —NH— |
| 309 | cycloalkylenyl | —C(═O)— | alkylenyl | — | —NH— |
| 310 | heterocyclenyl | —C(═O)— | alkylenyl | — | —NH— |
| 311 | — | —C(═O)NH— | alkylenyl | — | —NH— |
| 312 | alkylenyl | —C(═O)NH— | alkylenyl | — | —NH— |
| 313 | cycloalkylenyl | —C(═O)NH— | alkylenyl | — | —NH— |
| 314 | heterocyclenyl | —C(═O)NH— | alkylenyl | — | —NH— |
| 315 | — | —C≡C— | alkylenyl | — | —NH— |
| 316 | alkylenyl | —C≡C— | alkylenyl | — | —NH— |
| 317 | cycloalkylenyl | —C≡C— | alkylenyl | — | —NH— |
| 318 | heterocyclenyl | —C≡C— | alkylenyl | — | —NH— |
| 319 | alkylenyl | — | heterocyclenyl | — | —NH— |
| 320 | cycloalkylenyl | — | heterocyclenyl | — | —NH— |
| 321 | heterocyclenyl | — | heterocyclenyl | — | —NH— |
| 322 | — | —C(═O)— | heterocyclenyl | — | —NH— |
| 323 | alkylenyl | —C(═O)— | heterocyclenyl | — | —NH— |
| 324 | cycloalkylenyl | —C(═O)— | heterocyclenyl | — | —NH— |
| 325 | heterocyclenyl | —C(═O)— | heterocyclenyl | — | —NH— |
| 326 | — | —C(═O)NH— | heterocyclenyl | — | —NH— |
| 327 | alkylenyl | —C(═O)NH— | heterocyclenyl | — | —NH— |
| 328 | cycloalkylenyl | —C(═O)NH— | heterocyclenyl | — | —NH— |
| 329 | heterocyclenyl | —C(═O)NH— | heterocyclenyl | — | —NH— |
| 330 | — | —C≡C— | heterocyclenyl | — | —NH— |
| 331 | alkylenyl | —C≡C— | heterocyclenyl | — | —NH— |
| 332 | cycloalkylenyl | —C≡C— | heterocyclenyl | — | —NH— |
| 333 | heterocyclenyl | —C≡C— | heterocyclenyl | — | —NH— |
| 334 | alkylenyl | — | alkylenyl | heterocyclenyl | —NH— |
| 335 | cycloalkylenyl | — | alkylenyl | heterocyclenyl | —NH— |
| 336 | heterocyclenyl | — | alkylenyl | heterocyclenyl | —NH— |
| 337 | — | —C(═O)— | alkylenyl | heterocyclenyl | —NH— |
| 338 | alkylenyl | —C(═O)— | alkylenyl | heterocyclenyl | —NH— |
| 339 | cycloalkylenyl | —C(═O)— | alkylenyl | heterocyclenyl | —NH— |
| 340 | heterocyclenyl | —C(═O)— | alkylenyl | heterocyclenyl | —NH— |
| 341 | — | —C(═O)NH— | alkylenyl | heterocyclenyl | —NH— |
| 342 | alkylenyl | —C(═O)NH— | alkylenyl | heterocyclenyl | —NH— |
| 343 | cycloalkylenyl | —C(═O)NH— | alkylenyl | heterocyclenyl | —NH— |
| 344 | heterocyclenyl | —C(═O)NH— | alkylenyl | heterocyclenyl | —NH— |
| 345 | — | —C≡C— | alkylenyl | heterocyclenyl | —NH— |
| 346 | alkylenyl | —C≡C— | alkylenyl | heterocyclenyl | —NH— |
| 347 | cycloalkylenyl | —C≡C— | alkylenyl | heterocyclenyl | —NH— |
| 348 | heterocyclenyl | —C≡C— | alkylenyl | heterocyclenyl | —NH— |
| 349 | alkylenyl | — | heterocyclenyl | alkylenyl | —NH— |
| 350 | cycloalkylenyl | — | heterocyclenyl | alkylenyl | —NH— |
| 351 | heterocyclenyl | — | heterocyclenyl | alkylenyl | —NH— |
| 352 | — | —C(═O)— | heterocyclenyl | alkylenyl | —NH— |
| 353 | alkylenyl | —C(═O)— | heterocyclenyl | alkylenyl | —NH— |
| 354 | cycloalkylenyl | —C(═O)— | heterocyclenyl | alkylenyl | —NH— |
| 355 | heterocyclenyl | —C(═O)— | heterocyclenyl | alkylenyl | —NH— |
| 356 | — | —C(═O)NH— | heterocyclenyl | alkylenyl | —NH— |
| 357 | alkylenyl | —C(═O)NH— | heterocyclenyl | alkylenyl | —NH— |
| 358 | cycloalkylenyl | —C(═O)NH— | heterocyclenyl | alkylenyl | —NH— |
| 359 | heterocyclenyl | —C(═O)NH— | heterocyclenyl | alkylenyl | —NH— |
| 360 | — | —C≡C— | heterocyclenyl | alkylenyl | —NH— |
| 361 | alkylenyl | —C≡C— | heterocyclenyl | alkylenyl | —NH— |
| 362 | cycloalkylenyl | —C≡C— | heterocyclenyl | alkylenyl | —NH— |
| 363 | heterocyclenyl | —C≡C— | heterocyclenyl | alkylenyl | —NH— |
| 364 | alkylenyl | — | — | cycloalkylenyl | —NH— |
| 365 | cycloalkylenyl | — | — | cycloalkylenyl | —NH— |
| 366 | heterocyclenyl | — | — | cycloalkylenyl | —NH— |
| 367 | — | —C(═O)— | — | cycloalkylenyl | —NH— |
| 368 | alkylenyl | —C(═O)— | — | cycloalkylenyl | —NH— |
| 369 | cycloalkylenyl | —C(═O)— | — | cycloalkylenyl | —NH— |
| 370 | heterocyclenyl | —C(═O)— | — | cycloalkylenyl | —NH— |
| 371 | — | —C(═O)NH— | — | cycloalkylenyl | —NH— |
| 372 | alkylenyl | —C(═O)NH— | — | cycloalkylenyl | —NH— |
| 373 | cycloalkylenyl | —C(═O)NH— | — | cycloalkylenyl | —NH— |
| 374 | heterocyclenyl | —C(═O)NH— | — | cycloalkylenyl | —NH— |
| 375 | — | —C≡C— | — | cycloalkylenyl | —NH— |
| 376 | alkylenyl | —C≡C— | — | cycloalkylenyl | —NH— |
| 377 | cycloalkylenyl | —C≡C— | — | cycloalkylenyl | —NH— |
| 378 | heterocyclenyl | —C≡C— | — | cycloalkylenyl | —NH— |
| 379 | alkylenyl | — | alkylenyl | cycloalkylenyl | —NH— |
| 380 | cycloalkylenyl | — | alkylenyl | cycloalkylenyl | —NH— |
| 381 | heterocyclenyl | — | alkylenyl | cycloalkylenyl | —NH— |
| 382 | — | —C(═O)— | alkylenyl | cycloalkylenyl | —NH— |
| 383 | alkylenyl | —C(═O)— | alkylenyl | cycloalkylenyl | —NH— |
| 384 | cycloalkylenyl | —C(═O)— | alkylenyl | cycloalkylenyl | —NH— |
| 385 | heterocyclenyl | —C(═O)— | alkylenyl | cycloalkylenyl | —NH— |
| 386 | — | —C(═O)NH— | alkylenyl | cycloalkylenyl | —NH— |
| 387 | alkylenyl | —C(═O)NH— | alkylenyl | cycloalkylenyl | —NH— |
| 388 | cycloalkylenyl | —C(═O)NH— | alkylenyl | cycloalkylenyl | —NH— |
| 389 | heterocyclenyl | —C(═O)NH— | alkylenyl | cycloalkylenyl | —NH— |
| 390 | — | —C≡C— | alkylenyl | cycloalkylenyl | —NH— |
| 391 | alkylenyl | —C≡C— | alkylenyl | cycloalkylenyl | —NH— |
| 392 | cycloalkylenyl | —C≡C— | alkylenyl | cycloalkylenyl | —NH— |
| 393 | heterocyclenyl | —C≡C— | alkylenyl | cycloalkylenyl | —NH— |
| 394 | alkylenyl | — | heterocyclenyl | cycloalkylenyl | —NH— |
| 395 | cycloalkylenyl | — | heterocyclenyl | cycloalkylenyl | —NH— |
| 396 | heterocyclenyl | — | heterocyclenyl | cycloalkylenyl | —NH— |
| 397 | — | —C(═O)— | heterocyclenyl | cycloalkylenyl | —NH— |
| 398 | alkylenyl | —C(═O)— | heterocyclenyl | cycloalkylenyl | —NH— |
| 399 | cycloalkylenyl | —C(═O)— | heterocyclenyl | cycloalkylenyl | —NH— |
| 400 | heterocyclenyl | —C(═O)— | heterocyclenyl | cycloalkylenyl | —NH— |
| 401 | — | —C(═O)NH— | heterocyclenyl | cycloalkylenyl | —NH— |
| 402 | alkylenyl | —C(═O)NH— | heterocyclenyl | cycloalkylenyl | —NH— |
| 403 | cycloalkylenyl | —C(═O)NH— | heterocyclenyl | cycloalkylenyl | —NH— |
| 404 | heterocyclenyl | —C(═O)NH— | heterocyclenyl | cycloalkylenyl | —NH— |
| 405 | — | —C≡C— | heterocyclenyl | cycloalkylenyl | —NH— |
| 406 | alkylenyl | —C≡C— | heterocyclenyl | cycloalkylenyl | —NH— |
| 407 | cycloalkylenyl | —C≡C— | heterocyclenyl | cycloalkylenyl | —NH— |
| 408 | heterocyclenyl | —C≡C— | heterocyclenyl | cycloalkylenyl | —NH— |

In another embodiment, PROTAC Molecules are compounds of any one of Formulae XXIII-XXXIV, or a pharmaceutically acceptable salt or solvate thereof, wherein Q is a small molecule that binds to a target protein of interest.

In another embodiment, PROTAC Molecules are compounds of any one of Formulae XXIII-XXXIV, or a pharmaceutically acceptable salt or solvate thereof, wherein Q is a Hsp90 inhibitor, a kinase inhibitor, a MDM2 inhibitor, a compound targeting cytosolic signaling protein, a HDAC inhibitor, a human lysine methyltransferase inhibitor, an angiogenesis inhibitor, an immunosuppressive compound, or compound a targeting the aryl hydrocarbon receptor (AHR).

In another embodiment, PROTAC Molecules are compounds of any one of Formulae XXIII-XXXIV, or a pharmaceutically acceptable salt or solvate thereof, wherein Q binds to a kinase, a cytosolic signaling protein, e.g., FKBP12, a nuclear protein, a histone deacetylase, a lysine methyltransferase, a protein regulating angiogenesis, a protein regulating immune response, an aryl hydrocarbon receptor (AHR), a glucocorticoid receptor, or a transcription factor, e.g., SMARCA4, SMARCA2, TRIM24.

In another embodiment, PROTAC Molecules are compounds of any one of Formulae XXIII-XXXIV, or a pharmaceutically acceptable salt or solvate thereof, wherein Q binds to a kinase, e.g., a tyrosine kinase, e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYRO3, YES1, or ZAP70; a aserine/threonine kinase, e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR,NEK2, NEK3, NEK6, NEK7, NEK9, NEK11, PAK1, PAK2, PAK3, PAK4, PAK5, PAK6, PIM1, PIM2, PLK1, RIP2, RIPS, RSK1, RSK2, SGK2, SGK3, SIK1, STK33, TAO1, TAO2, TGF-beta, TLK2, TSSK1, TSSK2, ULK1, or ULK2; a cyclin dependent kinase, e.g., Cdk1-Cdk11, or a leucine-rich repeat kinase, e.g., LRRK2.

In another embodiment, PROTAC Molecules are compounds of any one of Formulae XXIII-XXXIV, or a pharmaceutically acceptable salt or solvate thereof, wherein Q binds to antennapedia homeodomain protein, BRCA1, BRCA2, CCAAT-enhanced-binding proteins, histones, polycomb-group proteins, high mobility group proteins, telomere binding proteins, FANCA, FANCD2, FANCE, FANCF, hepatocyte nuclear factors, Mad2, NF-kappa B, nuclear receptor coactivators, CREB-binding protein, p55, p107, p130, Rb proteins, p53, c-fos, c-jun, c-mdm2, c-myc, or c-rel.

In another embodiment, PROTAC Molecules are compounds of any one of Formulae XXIII-XXXIV, or a pharmaceutically acceptable salt or solvate thereof, wherein Q is a half-life extending moiety, see, e.g., Bech et al., ASC Med. Chem. Lett. 9:577-580 (2018), a fluorophore, or a dye.

### VII. Definitions

The term “a disease or condition wherein inhibition of CRBN ubiquitination provides a benefit” and the like pertains to a disease or condition in which CRBN ubiquitination is important or necessary, e.g., for the onset, progress, expression of that disease or condition, or a disease or a condition which is known to be treated by an CRBN ubiquitination inhibitor, e.g., thalidomide, lenalidomide, pomalidomide, and related analogs. Examples of such conditions include, but are not limited, cancer. One of ordinary skill in the art is readily able to determine whether a compound treats a disease or condition mediated by a CRBN ubiquitination inhibitor for any particular cell type, for example, by assays which conveniently can be used to assess the activity of particular compounds.

The terms “cereblon” or “CRBN” refers to a protein that is encoded by the CRBN gene in humans. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Angers et al., Nature 443:590-593 (2006)

The term “optional therapeutic agent” refers to a therapeutic agent different from a Compound of the Disclosure and that is known to treat the disease or condition of interest. For example when a cancer is the disease or condition of interest, the optional therapeutic agent can be a known chemotherapeutic drug, like taxol, or radiation, for example.

The term “disease” or “condition” denotes disturbances and/or anomalies that as a rule are regarded as being pathological conditions or functions, and that can manifest themselves in the form of particular signs, symptoms, and/or malfunctions. Compounds of the Disclosure are inhibitors of CRBN ubiquitination and can be used in treating or preventing diseases and conditions wherein the inhibition of CRBN ubiquitination provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and subject to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to a subject in a sequence and sufficiently close in time so as to provide the desired therapeutic effect and can act in concert. For example, a Compound of the Disclosure can be administered at the same time or sequentially in any order at different points in time as an optional therapeutic agent. A Compound of the Disclosure and the optional therapeutic agent can be administered separately, in any appropriate form and by any suitable route. When a Compound of the Disclosure and the optional therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an optional therapeutic agent treatment modality (e.g., radiotherapy), to a subject in need thereof. In various embodiments, a Compound of the Disclosure and the optional therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at about 1 minute to about 24 hours apart.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-Cl₂ alkyl, or the number of carbon atoms designated, e.g., a C₁alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-Cl₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylC₆carbonyl, alkylsulfonyl, arylsulfonyl, ureido, guanidino, carbamate, carboxy, alkoxycarbonyl, carboxyalkyl,—N(R^{50a})C(═O)R^{50b}, —N(R^{50a})S(═O)R^{50c}, —C(═O)R⁵¹, —S (═O)R⁵², or —S(═O)₂R⁵³; wherein:

R^{50a} is hydrogen or alkyl;

R^{50b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁aryl, or optionally substituted heteroaryl;

R^{50c} is alkyl, haloalkyl, optionally substituted cycloalkyl, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁aryl, or optionally substituted heteroaryl;

R⁵¹ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl;

R⁵² is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁aryl, or optionally substituted heteroaryl; and

R⁵³ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In one embodiment, the alkynyl is a C₂-C₆ alkynyl. In another embodiment, the alkynyl is a C₂-C₄ alkynyl. In another embodiment, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)CCH.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group or alkenyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In one embodiment, the haloalkyl group is a C₁-C₆ haloalkyl. In another embodiment, the haloalkyl group is a C₁-C₄ haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In one embodiment, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In one embodiment, the alkoxy is a C₁-C₆ alkoxy. In another embodiment, the alkoxy is a C₁-C₄ alkoxy. In another embodiment, the alkyl is a C₁-C₆ alkyl. In another embodiment, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)—, or —S—. The —O—, —N(H)—, —N(C₁-C₄ alkyl)-, or —S—can independently be placed at any interior position of the aliphatic hydrocarbon chain so long as each —O—, —N(H)—, —N(C₁-C₄ alkyl)—, and —S—group is separated by at least two —CH₂— groups. In one embodiment, one —CH₂— group is replaced with one —O—group. In another embodiment, two —CH₂— groups are replaced with two —O—groups. In another embodiment, three —CH₂—groups are replaced with three —O— groups. In another embodiment, four —CH₂— groups are replaced with four —O— groups. Non-limiting exemplary heteroalkyl groups include —CH₂OCH₃, —CH₂OCH₂CH₂CH₃, —CH₂CH₂CH₂OCH₃, —CH₂CH₂OCH₂CH₂OCH₂CH₃, —CH₂CH₂OCH₂CH₂OCH₂CH₂OCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl,—N(R^{50a})C(═O)R^{50b},—N(R^{50a})S(═O)₂R^{50c}, —C(═O)R⁵¹, —S (═O)R⁵², —S(═O)₂R⁵³, or —OR⁵⁴, wherein R^{50a}, R^{50b}, R^{50c}, R⁵², R⁵¹, and R⁵³ are as defined in connection with the term “optionally substituted alkyl” and R⁵⁴ is (hydroxy)alkyl or (amino)alkyl. The term optionally substituted cycloalkyl also includes cycloalkyl groups having fused optionally substituted aryl or optionally substituted heteroaryl groups such as

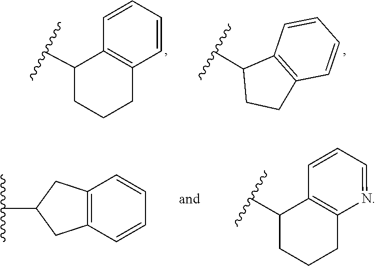 [SMILES: *C1CCCc2ccccc21 ; *C1CCc2ccccc21 ; *C1Cc2ccccc2C1 ; "and" ; *C1CCCc2ncccc21]

Non-limiting exemplary optionally substituted cycloalkyl groups include:

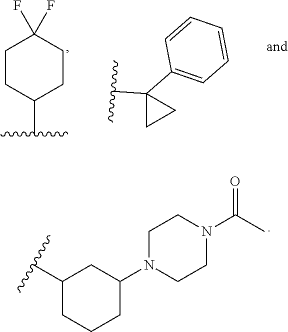 [SMILES: *C1CCC(F)(F)CC1 ; *C1(c2ccccc2)CC1 ; *C1CCCC(N2CCN(C(C)=O)CC2)C1]

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)—groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3 -dihydro- 1 H-pyrrolo [2,3 -c]pyridine, 2,3,4,5-tetrahydro-1 H-benzo [d] azepine, or 1,3,4,5-tetrahydro-2H-benzo [d] azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)—group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to12-membered cyclic group containing two rings and one or two nitrogen atoms. The heterocyclo can be linked to the rest of the molecule through any available carbon or nitrogen atom. Non-limiting exemplary heterocyclo groups include:

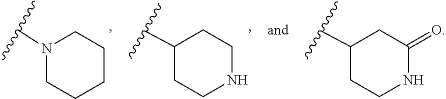

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, N(R^{50a})C(═O)R^{50b}, —N(R^{50a})S(═O)₂R^{50c}, —C(═O)R⁵¹, —S(═O)R⁵², —S(═O)₂R⁵³, or —OR⁵⁴, wherein R^{50a}, R^{50b}, R^{50c}, R⁵², R⁵¹, R⁵³, and R⁵⁴ are as defined in connection with the term “optionally substituted cycloalkyl.” Substitution may occur on any available carbon or nitrogen atom of the heterocyclo group. Non-limiting exemplary optionally substituted heterocyclo groups include:

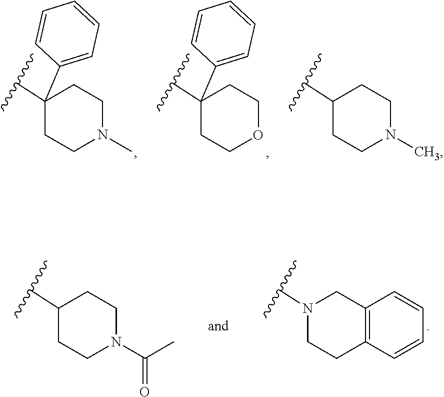

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{50a})C(═O)R^{50b}, —N(R^{50a})S(═O)R^{50c}, —C(═O)R⁵¹, —S(═O)R⁵², —S(═O)₂R⁵³, or —OR⁵⁴, wherein R^{50a}, R^{50b}, R^{50c}, R⁵², R⁵¹, R⁵³, and R⁵⁴ are as defined in connection with the term “optionally substituted cycloalkyl.”

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting xamples include: 2,3-dihydro-1H-inden-1-yl, 1,2,3,4-tetrahydronaphthalen-1-yl, 1,3,4,5-tetrahydro-2H-benzo [c] azepin-2-yl, 1,2,3,4-tetrahydroisoquinolin-1-yl, and 2-oxo-2,3,4,5-tetrahydro-1H-benzo [d] azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl,—N(R^{50a})C(═O)R^{50b}, —N(R^{50a})S(═O)₂R^{50c}, —C(═O)R⁵¹, —S(═O)R⁵², —S(═O)₂R⁵³, or —OR⁵⁴, wherein R^{50a}, R^{50b}, R^{50c}, R⁵², R⁵¹, R⁵³, and R⁵⁴ are as defined in connection with the term “optionally substituted cycloalkyl.”

In one embodiment, the optionally substituted heteroaryl has two substituents. In another embodiment, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “carboxyalkyl” as used herein by itself or as part of another group refers to an alkyl substituted with one carboxy group. In another embodiment, the alkyl is a C₁-C₄ alkyl. Non-limiting exemplary carboxyalkyl groups include —CH₂CO₂H and —CH₂CH₂CO₂H.

The term “(cyano)alkyl” as used herein by itself or as part of another group refers to an alkyl substituted with one, two, or three cyano groups. In one embodiment, the alkyl is substituted with one cyano group. In another embodiment, the alkyl is a C₁-C₆ alkyl. In another embodiment, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. In another embodiment, the alkyl is substituted with one optionally substituted cycloalkyl group. In another embodiment, the alkyl is substituted with two optionally substituted cycloalkyl groups. Non-limiting exemplary (cycloalkyl)alkyl groups include:

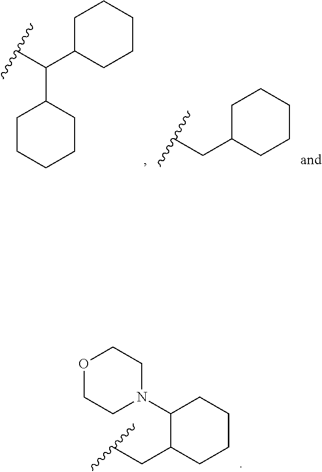 [SMILES: *C(C1CCCCC1)C1CCCCC1 ; *CC1CCCCC1 ; *CC1CCCCC1N1CCOCC1]

The term “sulfonamido” as used herein by itself or as part of another group refers to a radical of the formula —SO₂NR^{54a}R^{54b}, wherein R^{54a} and R^{54b} are each independently hydrogen, alkyl, optionally substituted cycloalkyl, optionally substituted heterocyclo, optionally substituted aryl, or optionally substituted heteroaryl; or R^{54a} and R^{54b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “ureido” as used herein by itself or as part of another group refers to a radical of the formula —NR^{51a}—C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R^{51c} taken together with the nitrogen to which they are attached form a 4- to 8-membered optionally substituted heterocyclo group. Non-limiting exemplary ureido groups include —NH—C(C═O)—NH2 and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R⁵³c are each independently hydrogen, alkyl, optionally substituted cycloalkyl, optionally substituted heterocyclo, optionally substituted aryl, or optionally substituted heteroaryl; or R^{52b} and R⁵²c taken together with the nitrogen to which they are attached form a 4- to 8-membered optionally substituted heterocyclo group; and R⁵³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom. Non-limiting exemplary (heterocyclo)alkyl groups include:

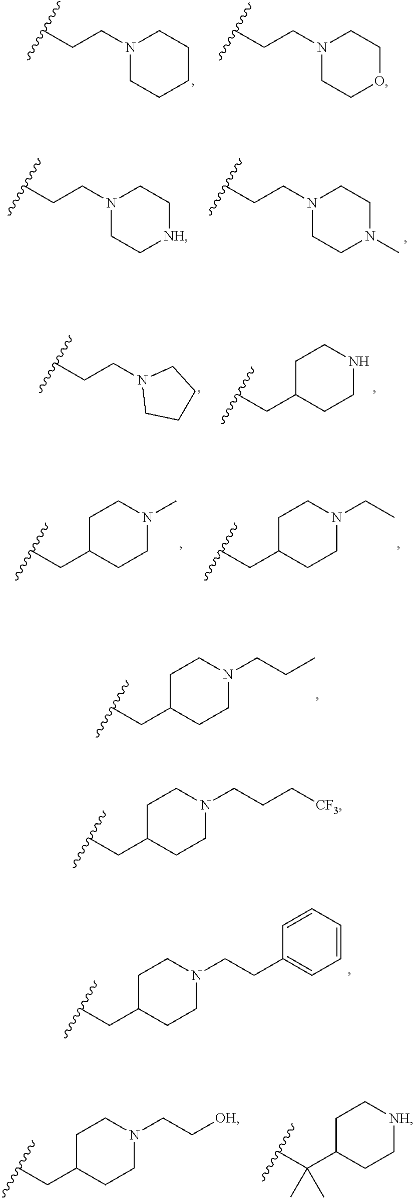

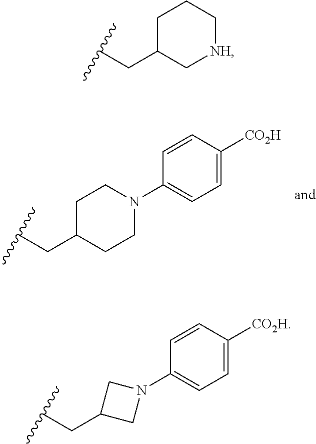 [SMILES: *CC1CCCNC1 ; *CC1CCN(c2ccc(C(=O)O)cc2)CC1 ; *CC1CN(c2ccc(C(=O)O)cc2)C1]

The term “carbamate” as used herein by itself or as part of another group refers to a radical of the formula —NR^{54a}—C(═O)—OR^{54b}, wherein R^{54a} is hydrogen or alkyl, and R^{54b} is hydrogen, alkyl, optionally substituted cycloalkyl, optionally substituted heterocyclo, optionally substituted aryl, or optionally substituted heteroaryl. A non-limiting exemplary carbamate group is —NH—(C═O)OtBu.

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl. Non-limiting exemplary (heteroaryl)alkyl groups include:

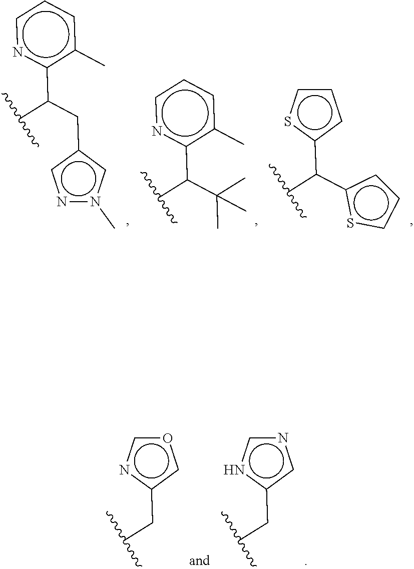

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “amido” as used herein by itself or as part of another group refers to a radical of formula —C(═O)NR^{60a}R^{60b}, wherein R^{60a} and R^{60b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{60a} and R^{60b} taken together with the nitrogen to which they are attached from a 4- to 8-membered optionally substituted heterocyclo group. In one embodiment, R^{60a} and R^{60b} are each independently hydrogen or C₁-C₆ alkyl.

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

In another embodiment, the amino is an “alkylamino,” i.e., an amino group wherein R^{55a} is C₁₋₆ alkyl and R^{55b} is hydrogen. In one embodiment, R⁵⁵a is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R^{55a} and R^{55b} are each independently C₁₋₆ alkyl. In one embodiment, R^{55a} and R^{55b} are each independently C₁-C₄ alkyl. Non-limiting exemplary dialkylamino groups include —N(CH₃)₂ and —N(CH₃)CH₂CH(CH₃)₂.

In another embodiment, the amino is a “hydroxyalkylamino,” i.e., an amino group wherein R^{55a} is (hydroxyl)alkyl and R^{55b} is hydrogen or C₁-C₄ alkyl.

In another embodiment, the amino is a “cycloalkylamino,” i.e., an amino group wherein R^{55a} is optionally substituted cycloalkyl and R^{55b} is hydrogen or C₁-C₄ alkyl.

In another embodiment, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂,and —N(CH₃)CH₂Ph.

In another embodiment, the amino is a “(cycloalkyl)alkylamino,” i.e., an amino group wherein R^{55a} is (cycloalkyl)alkyl and R^{55b} is hydrogen or C₁-C₄ alkyl. Non-limiting exemplary (cycloalkyl)alkylamino groups include:

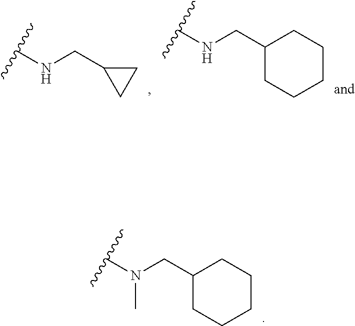

In another embodiment, the amino is a “(heterocyclo)alkylamino,” i.e., an amino group wherein R^{55a} is (heterocyclo)alkyl and R^{55b} is hydrogen or C₁-C₄ alkyl. Non-limiting exemplary (heterocyclo)alkylamino groups include:

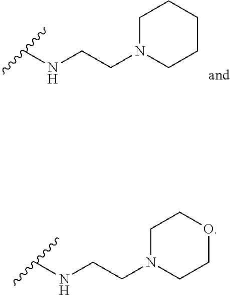 [SMILES: *NCCN1CCCCC1 ; *NCCN1CCOCC1]

The term “(amino)alkyl” as used herein by itself or as part of another group refers to an alkyl substituted with one amino group. In one embodiment, the amino group is —NH₂. In one embodiment, the amino group is an alkylamino. In another embodiment, the amino group is a dialkylamino. In another embodiment, the alkyl is a C₁-C₆ alkyl. In another embodiment, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

In the present disclosure, the term “alkylenyl” as used herein by itself or part of another group refers to a divalent form of an alkyl group, wherein the alkyl group is either unsubstituted or substituted with one or two groups independently selected from the group consisting of optionally substituted phenyl and optionally substituted 5- or 6-membered heteroaryl. In one embodiment, the alkylenyl is a divalent form of a C₁₋₁₂ alkyl, i.e., a C₁-Cl₂ alkylenyl. In one embodiment, the alkylenyl is a divalent form of a C₁₋₁₀ alkyl, i.e., a C₁-C₁₀ alkylenyl. In one embodiment, the alkylenyl is a divalent form of a C₁₋₈ alkyl, i.e., a C₁₋₈ alkylenyl. In one embodiment, the alkylenyl is a divalent form of an unsubstituted C₁₋₆ alkyl, i.e., a C₁-C₆ alkylenyl. In another embodiment, the alkylenyl is a divalent form of an unsubstituted C₁₋₄ alkyl, i.e., a C₁-C₄ alkylenyl. In another embodiment, the alkylenyl is a divalent form of a C₁₋₄ alkyl substituted with one or two optionally substituted phenyl groups. Non-limiting exemplary alkylenyl groups include —CH₂—, —CH₂CH₂—, —CH(Ph)-, —CH(Ph)CH₂—, —CH₂CH₂CH₂—, —CH(Ph)CH₂CH₂—, —CH₂(CH₂)₂CH₂—, —CH(CH₂)₃CH₂—, and —CH₂(CH₂)₄CH₂—.

The term “heteroalkylenyl” as used herein by itself or part of another group refers to a divalent form of a heteroalkyl group. In one embodiment, the heteroalkylenyl is a divalent form of a 3- to 20-membered heteroalkyl, i.e., a 3- to 20-membered heteroalkylenyl. In another embodiment, the heteroalkylenyl is a divalent form of a 3- to 10-membered heteroalkyl, i.e., a 3- to 10-membered heteroalkylenyl. In another embodiment, the heteroalkylenyl is a divalent form of a 3- to 8-membered heteroalkyl, i.e., a 3- to 8-membered heteroalkylenyl. In another embodiment, the heteroalkylenyl is a divalent form of a 3- to 6-membered heteroalkyl, i.e., a 3- to 6-membered heteroalkylenyl. In another embodiment, the heteroalkylenyl is a divalent form of a 3- or 4-membered heteroalkyl, i.e., a 3- or 4-membered heteroalkylenyl. In another embodiment, the heteroalkylenyl is a radical of the formula —(CH₂CH₂O)ᵤ₁— wherein u₁ is 1, 2, 3, 4, 5, or 6. Non-limiting exemplary heteroalkylenyl groups include —CH₂OCH₂—, —CH₂CH₂OCH₂CH₂O—, —CH₂OCH₂CH₂CH₂—, and —CH₂CH₂OCH₂CH₂OCH₂CH₂O—.

The term “heterocyclenyl” as used herein by itself or part of another group refers to a divalent form of an optionally substituted heterocyclo group. In another embodiment, the heterocyclenyl is a divalent form of a 4- to 14-membered heterocyclo group, i.e., a 4- to 14-membered heterocyclenyl. In another embodiment, the heterocyclenyl is a divalent form of a 4- to 10-membered heterocyclo group, i.e., a 4- to 10-membered heterocyclenyl. In another embodiment, the heterocyclenyl is a divalent form of a 4- to 8-membered heterocyclo group, i.e., a 4- to 8-membered heterocyclenyl. In one embodiment, the heterocyclenyl is a divalent form of an optionally substituted azetidine. In another embodiment, the heterocyclenyl is a divalent form of an optionally substituted piperidinyl. In another embodiment, the heterocyclenyl is a divalent form of an optionally substituted piperazinyl. Non-limiting exemplary heterocyclenyl groups include:

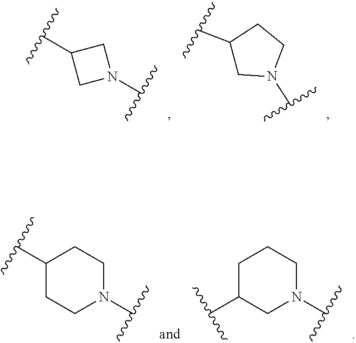 [SMILES: *C1CN(*)C1 ; *C1CCN(*)C1 ; *C1CCN(*)CC1 ; *C1CCCN(*)C1]

In another embodiment, the heterocyclenyl is a spiroheterocyclenyl.

The term “spiroheterocyclenyl” as used herein by itself or part of another group refers to a divalent form of a spiroheterocyclo. Non-limiting exemplary spiroheterocyclenyl groups include:

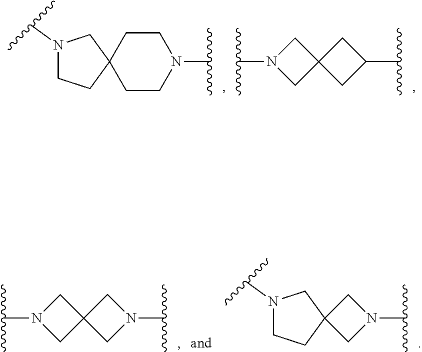

The term “cycloalkylenyl” as used herein by itself or part of another group refers to a divalent form of an optionally substituted C₄-C₆ cycloalkyl group. In one embodiment, the cycloalkylenyl is a 4-membered cycloalkylenyl. In another embodiment, the cycloalkylenyl is a 5-membered cycloalkylenyl. In another embodiment, the cycloalkylenyl is a 6-membered cycloalkylenyl. Non-limiting exemplary groups include:

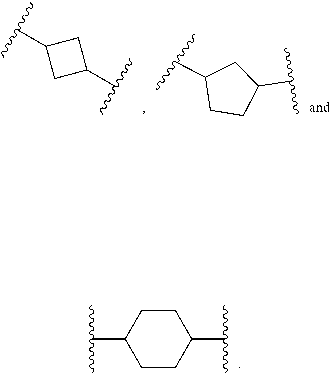 [SMILES: *C1CC(*)C1 ; *C1CCC(*)C1 ; *C1CCC(*)CC1]

The term “phenylenyl” as used herein by itself or part of another group refers to a divalent form of an optionally substituted phenyl group. Non-limiting examples include:

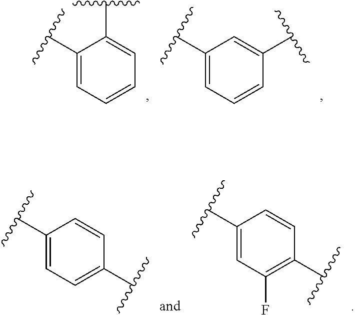

The term “heteroarylenyl” as used herein by itself or part of another group refers to a divalent form of an optionally substituted heteroaryl group, e.g., a 5- to 9-membered heteroarylenyl. In one embodiment, the heteroarylenyl is a 6-membered heteroarylenyl, e.g., heteroarylenyl derived from pyridine. In one embodiment, the heteroarylenyl is a bicyclic 9-membered heteroarylenyl. Exemplary non-limiting exemplary heteroarylenyl groups include:

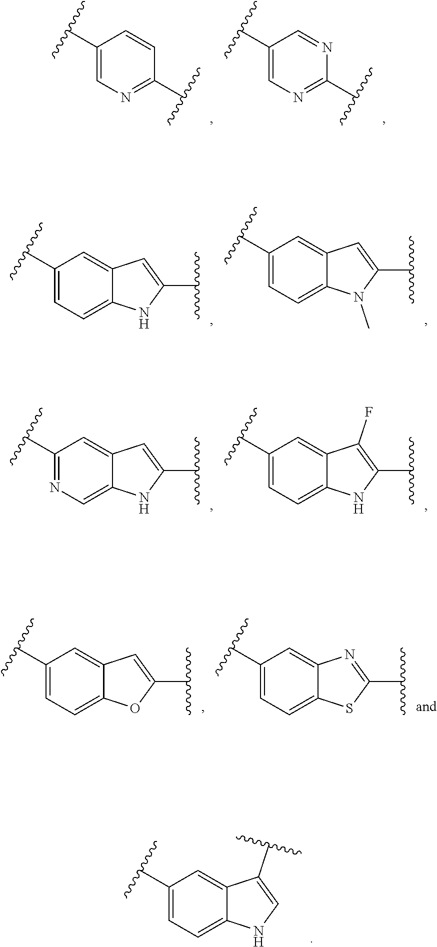

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, and ¹⁴C.

In one embodiment, provided is a composition wherein substantially all of the atoms at a position within the Compound of the Disclosure are replaced by an atom having a different atomic mass or mass number. In another embodiment, provided is a composition wherein a portion of the atoms at a position within the Compound of the disclosure are replaced, i.e., the Compound of the Disclosure is enriched at a position with an atom having a different atomic mass or mass number. For example, in some particular embodiments, the hydrogen atom at R³ in any one of Formulae I-IV can be replaced with a deuterium atom.

When a position of any one of Formulae I-IV, e.g., R³, is designated specifically as “H” or “hydrogen,” the position is understood to have hydrogen at its natural abundance isotopic composition.

When a position of any one of Formulae I-IV, e.g., R³, is designated specifically as “D” or “deuterium,” the position is understood to have deuterium at an abundance that is at least about 1000 times greater than the natural abundance of deuterium, which is about 0.015%.

Isotopically-labelled Compounds of the Disclosure can be prepared by methods known in the art.

Compounds of the Disclosure may contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S═1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number ±10%. Thus, “about 10” means 9 to 11.

### EXAMPLES

### Example 1

### Synthesis of tert-butyl 2-(2,6-dioxopiperidin-3-yl)-1,3-dioxo-1,2,3,5,7,8-hexahydro-6H-pyrrolo[3,4-g]isoquinoline-6-carboxylate (Cpd. No. 249) and

### 2-(2,6-dioxopiperidin-3-yl)-5,6,7,8-tetrahydro-1H-pyrrolo[3,4-g]isoquinoline-1,3(2H)-dione (Cpd. No. 241)

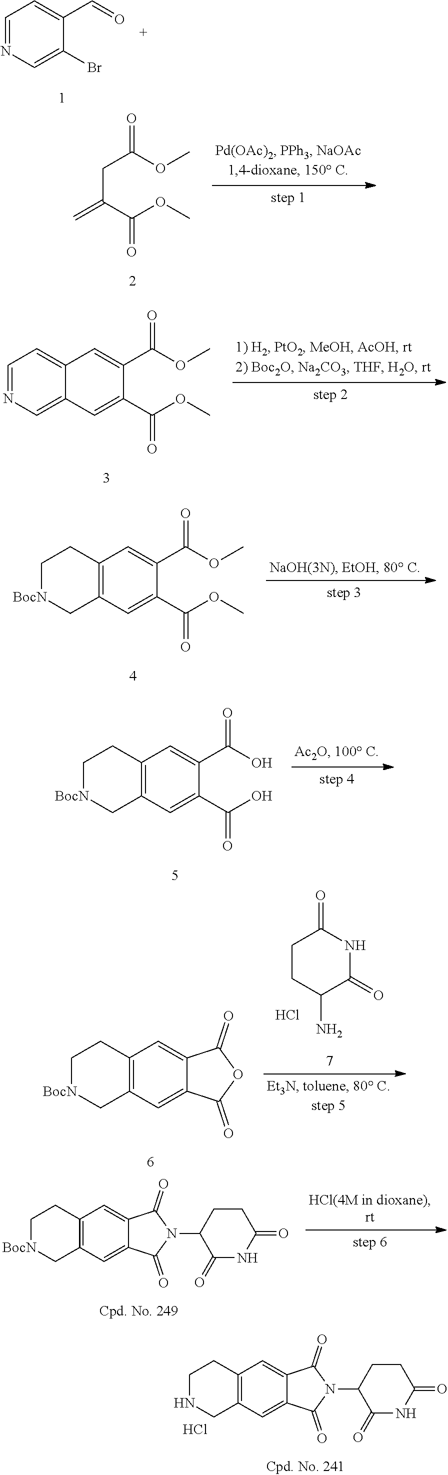

### Step 1: Synthesis of dimethyl isoquinoline-6,7-dicarboxylate (compound 3)

A mixture of 3-bromopyridine-4-carbaldehyde (1, 0.093 g, 0.5 mmol), dimethyl itaconate (2, 0.079 g, 0.5 mmol), Pd(OAc)₂ (0.0056 g, 0.025 mmol), PPh3 (0.013 g, 0.05 mmol) and NaOAc (0.123 g, 1.5 mmol) in dioxane (10mL) was placed in a 50 mL pressure vessel. After the system was flushed with argon, the reaction mixture was allowed to react at 150° C. for 24 h, and then the reaction mixture was cooled to room temperature. The reaction mixture was filtered through celite® to eliminate inorganic salts and washed by ethyl acetate. Removal of the solvent left a crude mixture which was purified by flash chromatography on silica gel (ethyl acetate-hexane) to give dimethyl isoquinoline-6,7-dicarboxylate (3, 0.082 g, 67%).

### Step 2: Synthesis of 2-(tert-butyl) 6,7-dimethyl 3,4-dihydroisoquinoline-2,6,7(1H)-tricarboxylate (compound 4)

Compound 3 (279.6 mg, 1.14 mmol) was dissolved in mixture solvent of methanol (4 mL) and acetic acid (0.2 mL). PtO₂ (30 mg) was added, and the reaction mixture was stirred under hydrogen at room temperature for 4h. The reaction mixture was filtered through celite®. The filtrate was collected and concentrated under reduced pressure to give the crude product.

The crude product was dissolved in mixture of THF (4 mL) and water (1 mL), and Na₂CO₃ (500 mg) and Boc₂O (500 mg, 2.28 mmol) were added to the mixture. The reaction mixture was stirred at room temperature for 2 h. The reaction mixture was concentrated under reduced pressure to remove the THF, and the crude mixture dissolved in water (5 mL) and ethyl acetate (10 mL). The organic layer was separated, washed with water and brine, dried (MgSO₄), concentrated under reduced pressure, and purified by flash chromatography on silica gel (ethyl acetate-hexane) to give compound 4 (130 mg).

### Step 3: Synthesis of 2-(tert-butoxycarbonyl)-1,2,3,4-tetrahydroisoquinoline-6,7-dicarboxylic acid (compound 5)

3N NaOH (0.37 mL, 1.12 mmol) was added to a solution of compound 4 (130 mg, 0.37 mmol) in EtOH (3.7 mL) and the resulting mixture heated at 80° C. for 2 h. The reaction was concentrated under reduced pressure and the crude mixture dissolved in water (5 mL) and ethyl acetate (10 mL) and then acidified using 1N HCl to pH ˜4 in an ice bath. The organic layer was separated and the aqueous layer was extracted with ethyl acetate two more times. The combined the organic layers were washed with brine (10 mL), dried (MgSO₄), and concentrated under reduced pressure. The crude product was used in the next step without further purification.

### Step 4: Synthesis of tert-butyl 1,3-dioxo-1,5,7,8-tetrahydrofuro[3,4-g]isoquinoline-6(3H)-carboxylate (compound 6)

Compound 5 (the crude product from step 3) was dissolved in acetic anyhydride (2 mL) and the reaction mixture was stirred at 100° C. for 3 h. The reaction mixture was cooled to room temperature, and 10 mL ethyl acetate was added. The reaction mixture waas washed with water and brine, dried (MgSO₄), concentrated under reduced pressure, and purified by flash chromatography on silica gel (ethyl acetate-hexane) to give compound 6 (123.1 mg).

### Step 5: Synthesis of tert-butyl 2-(2,6-dioxopiperidin-3-yl)-1,3-dioxo-1,2,3,5,7,8-hexahydro-6H-pyrrolo[3,4-g]isoquinoline-6-carboxylate (Cpd. No. 249)

Compound 6 (123.1 mg, 0.41 mmol), compound 7 (73.5 mg, 0.45 mmol) and Et₃N (0.17 mL, 1.23 mmol) were added to toluene (5 mL). The reaction mixture was stirred at 80° C. for 3 h and then cooled to room temperature. The reaction was concentrated under reduced pressure and the crude mixture dissolved in water (5 mL) and ethyl acetate (10 mL). The organic layer was separated, washed with water and brine, dried (MgSO₄), concentrated under reduced pressure, and purified by flash chromatography (ethyl acetate-hexane) to give Cpd. No. 249.

### Step 6: Synthesis of 2-(2,6-dioxopiperidin-3-yl)-5,6,7,8-tetrahydro-1H-pyrrolo[3,4-g]isoquinoline-1,3(2H)-dione (Cpd. No. 241)

Cpd. No. 249 (102.1 mg, 0.24 mmol) was added to 1 mL HCl (4M in 1,4-dioxane), and the mixture reaction mixture was stirred at room temperature for 2 h. The 1,4-dioxane was removed under reduced pressure to give Cpd. No. 241 as the HCl salt.

### Example 2

### Synthesis of tert-butyl 6-(2,6-dioxopiperidin-3-yl)-5,7-dioxo-3,5,6,7-tetrahydropyrrolo[3,4-f]-isoindole-2(1H)-carboxylate (Cpd. No. 189) and

### 2-(2,6-dioxopiperidin-3-yl)-6,7-dihydropyrrolo[3,4-f]isoindole-1,3 (2H,5H)-dione (Cpd. No. 181)

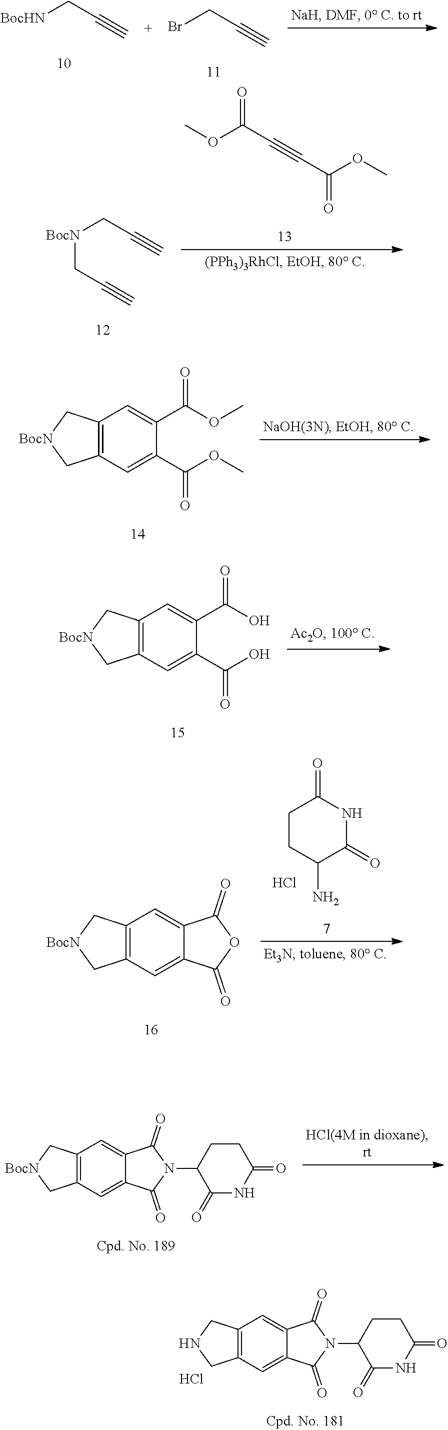

### Step 1: Synthesis of tert-butyl di(prop-2-yn-1-yl)carbamate (compound 12)

A solution of N-(tert-butyloxy)carbonyl propargylamine (compound 10; 33.36 g, 215 mmol) in 50 mL of DMF was treated portionwise (4 times) with 60% NaH (10.4 g) at 0° C. After stirring for 30 min at 25° C., 39 mL of an 80% solution of propargyl bromide (compound 11) in toluene was added. The reaction mixture was stirred for an additional 5 h at 25° C., and then quenched with the addition of ice-water. The mixture was extracted with Et₂O (×200mL), and the combined extracts were washed with saturated aqueous NaCl, dried (Na₂SO₄), concentrated in vacuo, and purified by flash chromatography on silica gel (ethyl acetate-hexane) to give compound 12.

### Step 2: Synthesis of 2-(tert-butyl) 5,6-dimethyl isoindoline-2,5,6-tricarboxylate (compound 14)

A solution of compound 12 (10.4 g, 53.9 mmol) and dimethyl acetylenedicarboxylate (compound 13, 30.7 g, 216 mmol) in 110 mL of absolute EtOH was degassed by bubbling N₂ through the solution for 10 min. To this solution was added 1.0 g (0.02 equiv) of Wilkinson's catalyst ((Ph₃P)₃RhCl) at 25° C. After being warmed at reflux for 18 h, the reaction mixture was cooled to 25° C. and concentrated in vacuo. The resulting brown residue was diluted in 200 mL of Et₂O, and the precipitate was removed by filtration over Celite®. The filtrate was concentrated and the crude product purified by column chromatography on silica gel (20% EtOAc/hexane) to give 4.60 g (26%) of compound 14.

The remaining steps for synthesizing Cpd. No. 181 (as the HCl salt) are essentially the same as Steps 3-6 described above in EXAMPLE 1.

### Example 3

### Synthesis of 4-(4-(6-(2,6-dioxopiperidin-3-yl)-5,7-dioxo-3,5,6,7-tetrahydropyrrolo[3,4-f]isoindol-2(1H)-yl)piperidin-1-yl)benzoic acid (Cpd. No. 828)

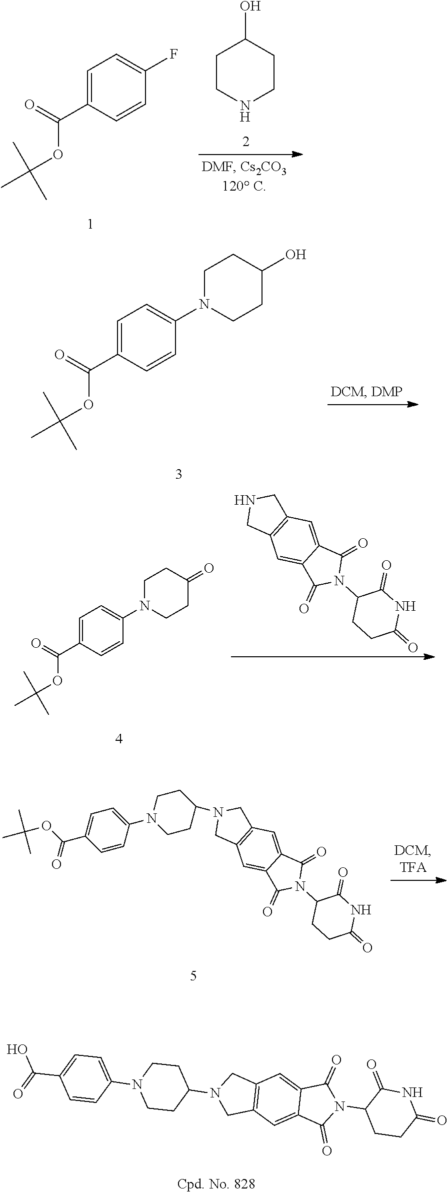

Compound 1 (1.0 eq) and compound 2 (1.5 eq) were dissolved in DMF, and Cs₂CO₃ (4 eq) was added. The reaction mixture was stirred overnight at 120° C. The reaction was partitioned between EtOAc and H₂O, and the organic layer was washed with brine. The concentrated crude product was purified on a Combiflash chromatography system using EtOAc/hexane as the eluent to give compound 3 in about 60% yield. Compound 3 demonstrated high UV absorption at 280 nm, but low absorption at 254 nm.

Compound 3 (1.0 eq) was dissolved in DCM, and Dess-Martin reagent (1.3 eq) was added. The reaction mixture was stirred at rt for 4 h. The reaction was partitioned between EtOAc and H₂O, and the organic layer was washed with brine. The concentrated crude product was purified on a Combiflash chromatography system using EtOAc/hexane as the eluent to give compound 4 in about 85% yield.

Cpd. No 181 (see Example 2) and TEA (1.5 eq) were dissolved in DCE. Compound 4 and AcOH (4 eq) were added. The mixture was stirred overnight. NaB(OAc)₃H (3 eq) was added and the reaction was complete in about 3 h. The reaction mixture was concentrated with silica gel and purified on a Combiflash chromatography

Compound 5 was dissolved in DCM and TFA (20×) was added. THe solvent and

TFA were removed to give Cpd. No. 828.

### Example 4

### Synthesis of 4-(4-((6-(2,6-dioxopiperidin-3-yl)-5,7-dioxo-3,5,6,7-tetrahydropyrrolo[3,4-f]isoindol-2(1H)-yl)methyl)piperidin-1-yl)benzoic acid (Cpd. No. 830)

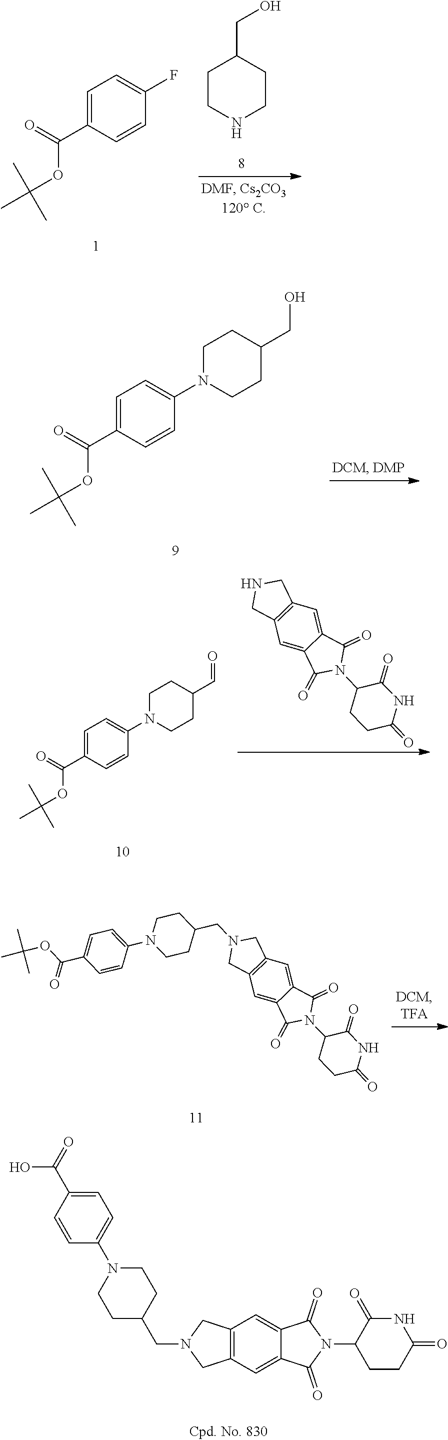

Compound 1 (1.0 eq) and compound 8 (1.5 eq) were dissolved in DMF, and Cs₂CO₃ (4 eq) was added. The reaction mixture was stirred overnight at 120° C. The reaction was partitioned between EtOAc and H₂O, and the organic layer was washed with brine. The concentrated crude product was purified on a Combiflash chromatography system using EtOAc/hexane as the eluent to give compound 9 in about 60% yield. Compound 9 demonstrated high UV absorption at 280 nm, but low absorption at 254 nm.

Compound 9 (1.0 eq) was dissolved in DCM, and Dess-Martin reagent (1.3 eq) was added. The reaction mixture was stirred at rt for 4 h. The reaction was partitioned between EtOAc and H₂O, and the organic layer was washed with brine. The concentrated crude product was purified on a Combiflash chromatography system using EtOAc/hexane as the eluent to give compound 10 in about 85% yield.

Compound 18 (see Example 21) and TEA (1.5 eq) were dissolved in DCE. Compound 10 and AcOH (4 eq) were added. The mixture was stirred overnight. NaB(OAc)₃H (3 eq) was added and the reaction was complete in about 3 h. The reaction mixture was concentrated with silica gel and purified on a Combiflash chromatography system using DCM/MeOH (5%) as the eluent to give compound 11.

Compound 11 was dissolved in DCM and TFA (20×) was added. The solvent and TFA were removed to give Cpd. No. 830.

### Example 5

### Synthesis of 2-(2,6-dioxopiperidin-3-yl)-6-(piperidin-4-yl)-6,7-dihydropyrrolo[3,4-f]isoindole-1,3(2H,5H)-dione (Cpd. No. 192)

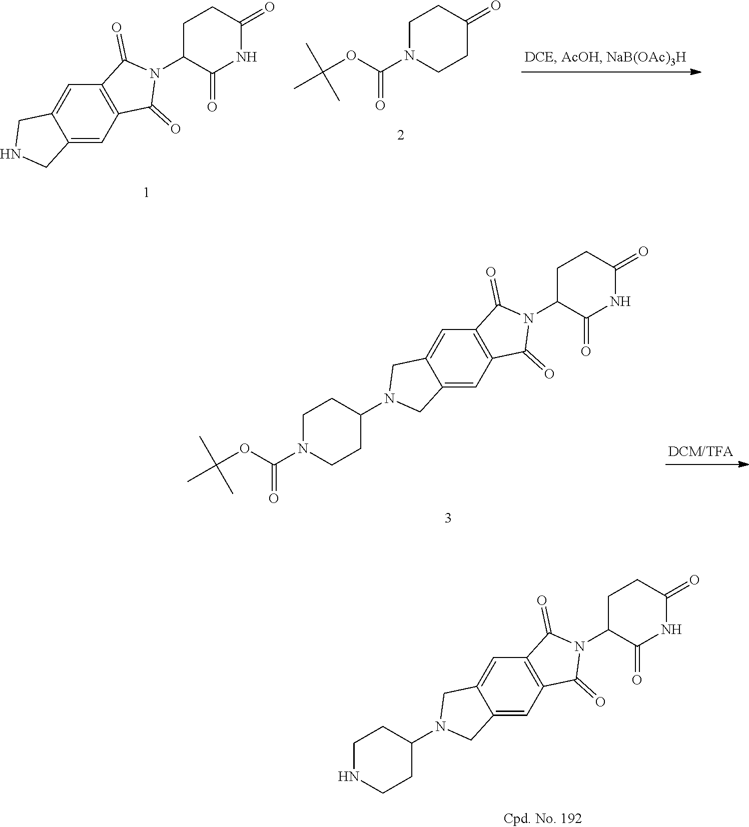 [SMILES: O=C1CCC(N2C(=O)c3cc4c(cc3C2=O)CNC4)C(=O)N1 ; CC(C)(C)OC(=O)N1CCC(=O)CC1 ; CC(C)(C)OC(=O)N1CCC(N2Cc3cc4c(cc3C2)C(=O)N(C2CCC(=O)NC2=O)C4=O)CC1 ; O=C1CCC(N2C(=O)c3cc4c(cc3C2=O)CN(C2CCNCC2)C4)C(=O)N1]

Compound 1 (1.0 eq) was dissolved in DCE (10×), and compound 2 (2.0 eq) and AcOH (3 eq.) were added. The mixture was stirred at rt for 2 h. Molecular sieves (4 angstrom) (3×) were added, and the mixture was stirred for 12 h. NaB(OAc)₃H (3.0 eq) was added, and the mixture was stirred at rt overnight. The reaction was concentrated and purified on a Combiflash chromatography system using MeOH/DCM as the eluent to provide compound 3 in 70% yield. Compound 3 was dissolved in 10X DCM, and TFA (2×) was added. The reaction mixture was stirred at rt for 2 h. The solvent was distilled and dried on the lyophilizer overnight to give Cpd. No. 192.

### Example 6

### Synthesis of 3 -(1-oxo-6-(piperidin-4-ylmethyl)-3,5,6,7-tetrahydropyrrolo[3,4-f]isoindol-2(1H)-yl)piperidine-2,6-dione (Cpd. No. 843)

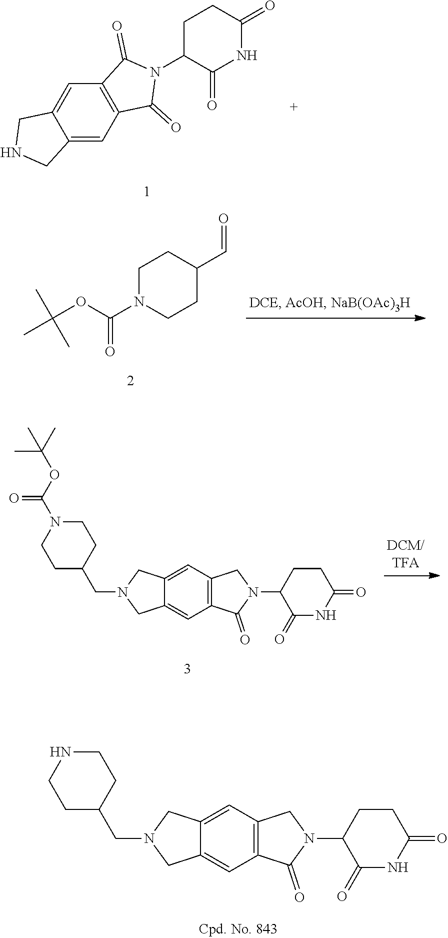 [SMILES: O=C1CCC(N2C(=O)c3cc4c(cc3C2=O)CNC4)C(=O)N1 ; CC(C)(C)OC(=O)N1CCC(C=O)CC1 ; CC(C)(C)OC(=O)N1CCC(CN2Cc3cc4c(cc3C2)C(=O)N(C2CCC(=O)NC2=O)C4)CC1 ; O=C1CCC(N2Cc3cc4c(cc3C2=O)CN(CC2CCNCC2)C4)C(=O)N1]

Compound 1 (1.0 eq) was dissolved in DCE (10×), and compound 2 (2.0 eq) and AcOH (3 eq.) were added. The mixture was stirred at rt for 2 h. Molecular sieves (4 angstrom) (3×) were added, and the mixture was stirred for 12 h. NaB(OAc)₃H (3.0 eq) was added, and the mixture was stirred at rt overnight. The reaction was concentrated and purified on a Combiflash chromatography system using MeOH/DCM as the eluent to give compound 3 in 90% yield. Compound 3 was dissolved in 10× DCM, and TFA (2×) was added. The reaction mixture was stirred at rt for 2 h. The solvent was removed and the product was dried on the lyophilizer overnight to give Cpd. No. 843.

### Example 7

### Synthesis of 2-(2,6-dioxopiperidin-3-yl)-1,3-dioxo-1,2,3,5,6,7-exahydrocyclopenta[f]isoindole-6-carbaldehyde (Cpd. No. 851)

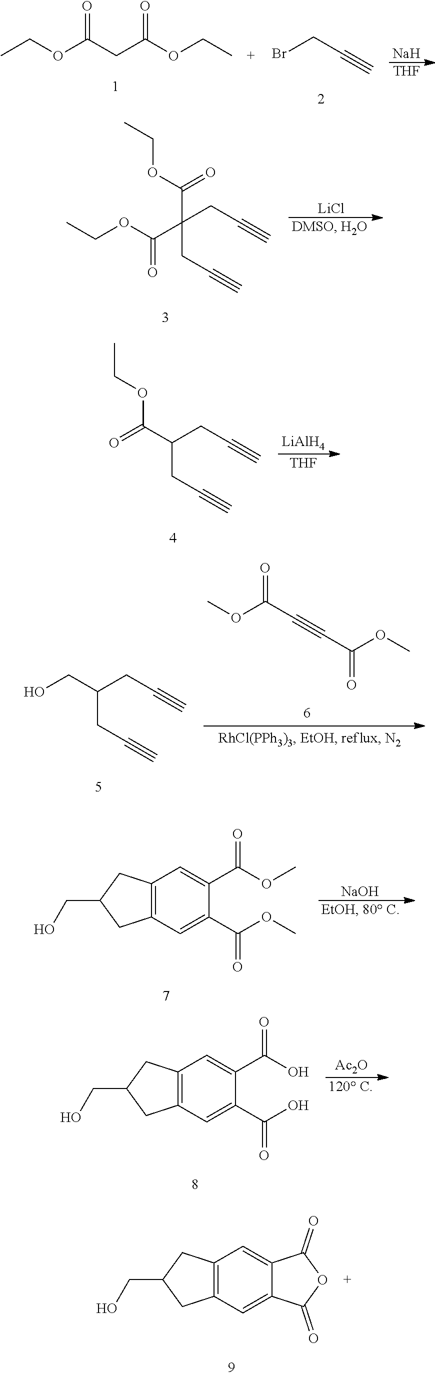

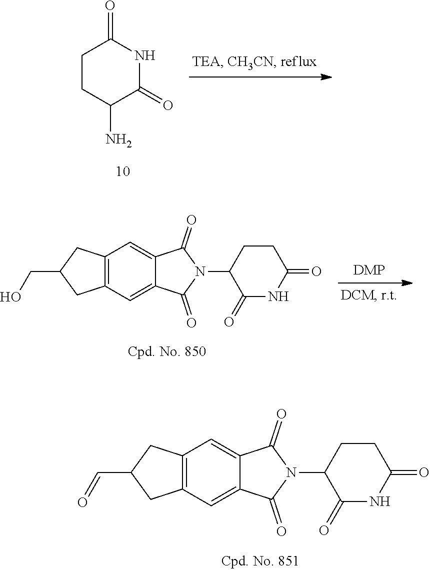 [SMILES: NC1CCC(=O)NC1=O ; O=C1CCC(N2C(=O)c3cc4c(cc3C2=O)CC(CO)C4)C(=O)N1 ; O=CC1Cc2cc3c(cc2C1)C(=O)N(C1CCC(=O)NC1=O)C3=O]

### Step 1: Synthesis of diethyl 2,2-di(prop-2-yn-1-yl)malonate

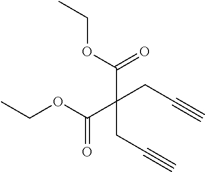 [SMILES: C#CCC(CC#C)(C(=O)OCC)C(=O)OCC]

To a suspension of sodium hydride (60% wt in mineral oil, 4.22 g, 105.5 mmol) in dry THF (100 mL) stirring at −10° C., dimethyl malonate (6.0 mL, 52.5 mmol) was added dropwise over 10 min. The reaction mixture was stirred at −10° C. for 5 min, and then propargyl bromide (80% wt. in toluene, 12.0 mL, 107.7 mmol) was added dropwise. The reaction mixture was warmed to 25° C. and stirred for 20 h. The reaction mixture was then poured into H₂O (50 mL) and Et₂O (50 mL), and the layers were separated. The aq layer was extracted with Et₂O (3×50 mL). The combined organic phases were washed with brine (50 mL), dried over MgSO₄, filtered, and concentrated on a rotary evaporator leaving a white solid. The solid was recrystallized from ethyl acetate and hexanes resulting in 9.44 g of a crystalline white solid (84% yield).

### Step 2: Synthesis of ethyl 2-(prop-2-yn-1-yl)pent-4-ynoate

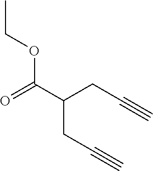

Dimethyl 2,2-di(2-propynyl)malonate (4.70 g, 22.6 mmol) and lithium chloride (2.95 g, 69.7 mmol) were dissolved in a solution of H₂O (1.0 mL, 55.5 mmol) and DMSO (40 mL). This solution was then heated to reflux for 1 h. After cooling, the reaction mixture was poured into CHCl₃ (40 mL) and H₂O (40 mL). The layers were separated and the aq layer was extracted with CHCl₃ (3×40 mL). The combined organic layers were washed with H₂O (50 mL) and brine (50 mL), dried, filtered through silica gel, and concentrated, leaving a yellow oil. The crude oil was purified by flash chromatography on a silica gel column using 20% EtOAc in hexanes as the eluent resulting in 3.06 g of a pale yellow oil (90% yield).

### Step 3: Synthesis of ethyl 2-(prop-2-yn-1-yl)pent-4-yn-1-ol

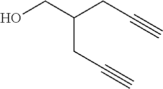

To a suspension of lithium aluminum hydride (1.25 g, 33.0 mmol) in dry THF (40 mL) stirring at -10° C. was added a solution of methyl 2-(2-propynyl)-4-pentynoate (3.06 g, 20.4 mmol) in dry THF (10 mL). The reaction mixture was allowed to warm to 25° C. and stirred for 12 h. The reaction mixture was then quenched through the dropwise addition of H₂O (1.25 mL), an aq 10% NaOH solution (1.25 mL), and then additional H₂O (3.75 mL). The reaction mixture was then stirred for 30 min until the suspended solids turned white. The mixture was then filtered, and the solids were washed with diethyl ether (100 mL). The resulting solution was concentrated on a rotary evaporator yielding a pale yellow oil. The crude oil was purified by flash chromatography on a silica gel column using 10% EtOAc in hexanes as the eluent, resulting in 1.95 g of a clear oil (78% yield).

### Step 4: Synthesis of dimethyl 2-(hydroxymethyl)-2,3-dihydro-1H-indene-5,6-dicarboxylate

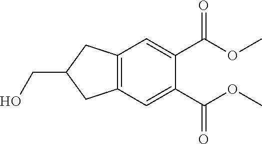 [SMILES: COC(=O)c1cc2c(cc1C(=O)OC)CC(CO)C2]

A solution of 5 and dimethyl acetylenedicarboxylate (6, 30.7 g, 216 mmol) in 110 mL of absolute EtOH was degassed by bubbling N₂ through the solution for 10 min. To this was added 1.0 g (0.02 equiv) of Wilkinson's catalyst ((Ph₃P)₃RhCl) at 25° C. After being warmed at reflux for 18 h, the reaction mixture was cooled to 25° C. and then concentrated in vacuo. The resulting brown residue was diluted in 200 mL of Et₂O, and the precipitate was removed by filtration over Celite®. The filtrate was concentrated and the crude product purified by column chromatography (20% EtOAc/hexane) to give 4.60g (26%) of compound 7.

### Step 5: Synthesis of 2-(hydroxymethyl)-2,3-dihydro-1H-indene-5,6-dicarboxylic acid

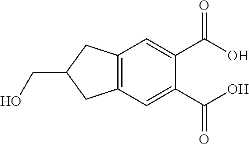 [SMILES: O=C(O)c1cc2c(cc1C(=O)O)CC(CO)C2]

NaOH (3N) was added to a solution of 7 in EtOH and stirred at 80° C. for 4 h. Then the EtOH was removed under reduced pressure, the pH was adjusted to acidity with 2 M HCl and the mixture was extracted with EtOAc. The solvent was removed to afford the product 8 which was used without further purification.

### Step 6: Synthesis of 6-(hydroxymethyl)-6,7-dihydro-1H-indeno[5,6-c]furan-1,3(5H)-dione

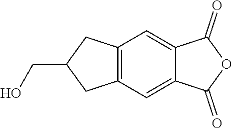 [SMILES: O=C1OC(=O)c2cc3c(cc21)CC(CO)C3]

The mixture of 8 in Ac₂O was stirred at 120° C. for 6 hours. All volatiles were removed and the residue was chromatographed on silica gel to afford 9.

### Step 7: Synthesis of 2-(2,6-dioxopiperidin-3-yl)-6-(hydroxymethyl)-6,7-5,7[f]isoindole-1,3(2H,5H)-dione (Cpd. No. 850)

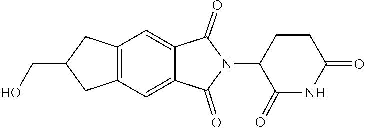 [SMILES: O=C1CCC(N2C(=O)c3cc4c(cc3C2=O)CC(CO)C4)C(=O)N1]

To a solution of 9 and 10 in toluene was added TEA (3 eq.). The mixture was stirred at reflux for 8 hours. All volatiles were removed and the residue was chromatographed on silica gel to afford Cpd. No. 850.

### Step 8: Synthesis of 2-(2,6-dioxopiperidin-3-yl)-1,3-dioxo-1,2,3,5,6,7-hexahydrocyclopenta[f]isoindole-6-carbaldehyde (Cpd. No. 851)

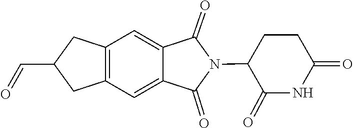 [SMILES: O=CC1Cc2cc3c(cc2C1)C(=O)N(C1CCC(=O)NC1=O)C3=O]

To a solution of Cpd. No. 850 in DCM was added DMP (1.2 eq.). The reaction mixture was stirred at reflux for 4 hours. All volatiles were removed and the residue was chromatographed on silica gel to afford 2-(2,6-dioxopiperidin-3-yl)-1,3-dioxo-1,2,3 ,5,6,7-hexahydrocyclopenta[f]isoindole-6-carbaldehyde (Cpd. No. 851). ESI-MS: 326.09.

### Example 8

### Synthesis of 2-(2,6-dioxopiperidin-3-yl)-5,7-dihydrocyclopenta[f]isoindole- 1,3 ,6(2H)-trione (Cpd. No. 853)

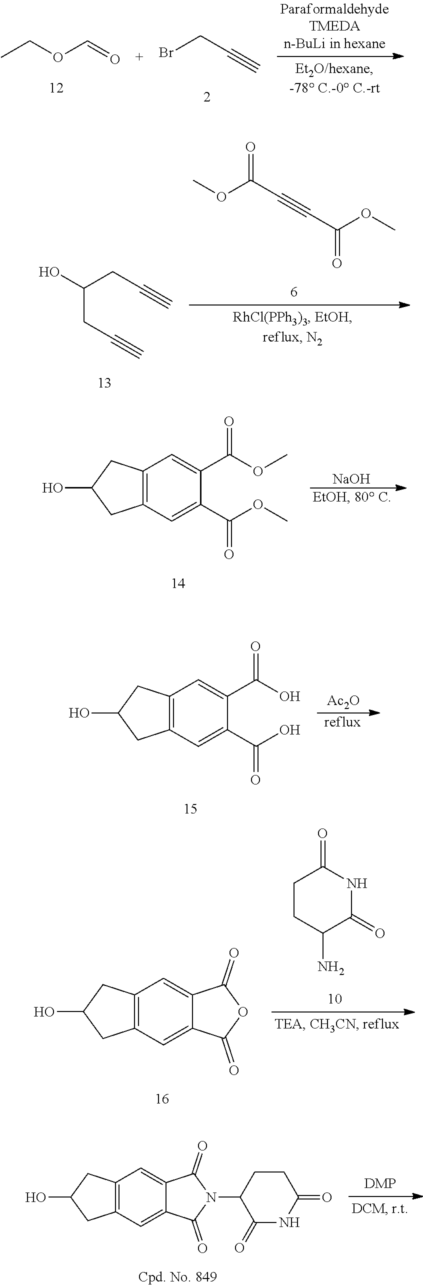

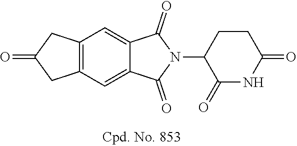 [SMILES: O=C1Cc2cc3c(cc2C1)C(=O)N(C1CCC(=O)NC1=O)C3=O]

### Step 1: Synthesis of hepta-1,6-diyn-4-ol

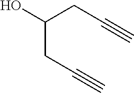

To a solution of n-BuLi in hexane (6.2 eq., 75 mL) in Et₂O/hexane (100 mL) was added TMEDA (7.5 mL) and 2 (3.1 eq.) by dropwise at −78° C. The reaction mixture was stirred at −78° C. for 40 min, and then 12 in THF (20 mL) was added dropwise with 10 min. The reaction mixture was warmed to 25° C. and stirred for 2 h. The reaction mixture was then cooled to −78° C. and added 20 mL THF and Paraformaldehyde (13.5 g) in one portion. Then, the mixture was stirred at r.t. overnight. The mixture was added ice-cold NH₄Cl solution and extracted with Et₂O (3×50 mL). The combined organic phases were washed with brine (50 mL), dried over MgSO₄, filtered, and concentrated on a rotary evaporator leaving a white solid. The solid was recrystallized from ethyl acetate and hexanes resulting in 13.

### Step 2: Synthesis of dimethyl 2-hydroxy-2,3-dihydro-1H-indene-5,6-dicarboxylate

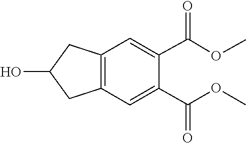 [SMILES: COC(=O)c1cc2c(cc1C(=O)OC)CC(O)C2]

A solution of 13 and dimethyl acetylenedicarboxylate (6, 30.7 g, 216 mmol) in 110 mL of absolute EtOH was degas sed by bubbling N₂ through the solution for 10 min. To this was added 1.0 g (0.02 equiv) of Wilkinson's catalyst ((Ph₃P)₃RhCl) at 25° C. After being warmed at reflux for 18 h, the reaction mixture was cooled to 25° C. and then concentrated in vacuo. The resulting brown residue was diluted in 200 mL of Et₂O, and the precipitate was removed by filtration over Celite®. The filtrate was concentrated and the crude product purified by column chromatography (20% EtOAc/hexane) to give 4.60g (26%) of compound 14.

### Step 3: Synthesis of 2-hydroxy-2,3-dihydro-1H-indene-5,6-dicarboxylic acid

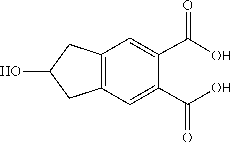 [SMILES: O=C(O)c1cc2c(cc1C(=O)O)CC(O)C2]

NaOH (3N) was added to a solution of 14 in EtOH and stirred at 80° C. for 4 h. Then the EtOH was removed under reduced pressure, the pH was adjusted to acidity with 2 M HCl and the mixture was extracted with EtOAc. The solvent was removed to afford the product 15 which was used without further purification.

### Step 4: Synthesis of 6-hydroxy-6,7-dihydro-1H-indeno[5,6-c]furan-1,3(5H)-dione

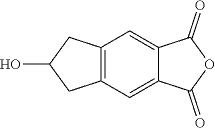 [SMILES: O=C1OC(=O)c2cc3c(cc21)CC(O)C3]

The mixture of 15 in Ac₂O was stirred at 120° C. for 6 hours. All volatiles were removed and the residue was chromatographed on silica gel to afford 16.

### Step 5: Synthesis of 2-(2,6-dioxopiperidin-3-yl)-6-hydroxy-6,7-dihydrocyclopenta[f]isoindole-1,3(2H,5H)-dione (Cpd. No. 849)

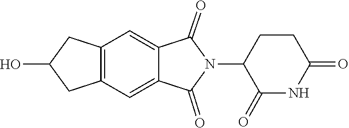 [SMILES: O=C1CCC(N2C(=O)c3cc4c(cc3C2=O)CC(O)C4)C(=O)N1]

To a solution of 16 and 10 in toluene was added TEA (3 eq.). The mixture was stirred at reflux for 8 hours. All volatiles were removed and the residue was chromatographed on silica gel to afford Cpd. No. 849.

### Step 6: Synthesis of 2-(2,6-dioxopiperidin-3-yl)-5,7-dihydrocyclopenta[f]isoindole-1,3,6(2H)-trione

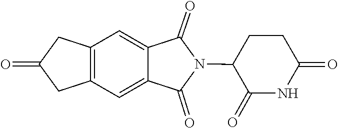 [SMILES: O=C1Cc2cc3c(cc2C1)C(=O)N(C1CCC(=O)NC1=O)C3=O]

To a solution of Cpd. No. 849 in DCM was added DMP (1.2 eq.). The reaction mixture was stirred at reflux for 4 hours. All volatiles were removed and the residue was chromatographed on silica gel to afford intermediate 2-(2,6-dioxopiperidin-3-yl)-5,7-dihydrocyclopenta[f]isoindole-1,3,6(2H)-trione (Cpd. No. 853). ESI-MS: 312.07.

### Example 9

### Synthesis of 2-(2,6-dioxopiperidin-3-yl)-6,7,8,9-tetrahydroazepino [4,5-f]isoindole-1,3(2H,5H)-dione (Cpd. No. 855)

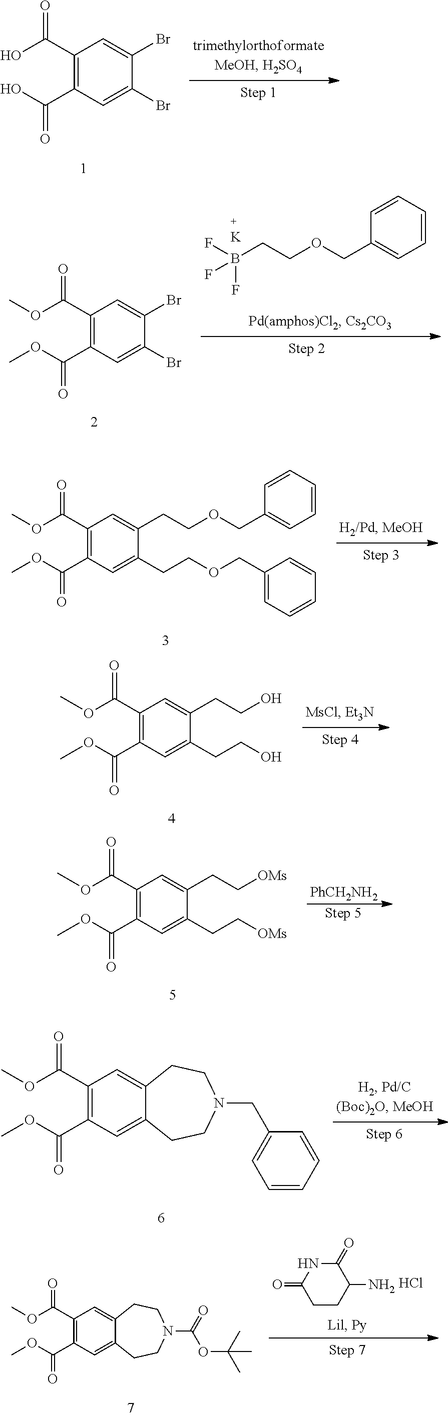

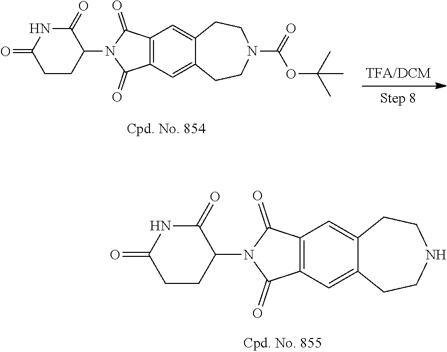 [SMILES: CC(C)(C)OC(=O)N1CCc2cc3c(cc2CC1)C(=O)N(C1CCC(=O)NC1=O)C3=O ; O=C1CCC(N2C(=O)c3cc4c(cc3C2=O)CCNCC4)C(=O)N1]

### Step 1: Synthesis of Dimethyl 4,5-dibromophthalate (Compound 2)

To a solution of 4,5-dibromophthalic acid (5 g) in MeOH (25 mL) and trimethyl orthoformate (25 mL) was added conc. H₂SO₄ (2.20 mL) at room temperature, and the reaction was refluxed overnight (about 12 h), solvent was removed under vacuum, EtOAc (100 mL) and sat. aq. NaHCO₃ (100 mL) was added. The products were extracted with EtOAc (50 mL6×3), and the combined organic extracts were washed with brine, dried (Na₂SO₄), and concentrated in vacuo. The residue was used in the next step without further purification. ¹H NMR (400 MHz, CDCl₃) δ 3.91 (m, 6H), 7.97 (s, 2H).

### Step 2: Synthesis of dimethyl 4,5-bis(2-(benzyloxy)ethyl)phthalate (Compound 3)

Dimethyl 4,5-dibromophthalate (1.1 g, 3.13 mmol, 1.0 equiv), potassium (2-(benzyloxy) ethyl)trifluoroborate (1.66 g, 6.88 mmol, 2.2 equiv) and Cs₂CO₃ (4.58 g, 14.1 mmol, 4.5 equiv) was dissolved in toluene (25 mL)/water (12.5 mL). Pd(amphos)Cl₂ (325 mg, 0.46 mmol, 0.15 equiv) was added and the reaction mixture was stirred overnight (12 h) at 100° C. under N₂. After cooling to room temperature, the reaction mixture was extracted with EtOAc (20 mL×3), washed with brine, dried (Na₂SO₄), and concentrated in vacuo. The resulting crude product was purified by flash column chromatography on silica gel (petroleum ether/EtOAc=20:1 to 1:1) to give dimethyl 4,5-bis(2-(benzyloxy)ethyl)phthalate as colorless oil (910 mg, 62%). ¹H NMR (400 MHz, CDCl₃) δ 7.61 (s, 2H), 7.37-7.29 (m, 10H), 4.50 (s, 4H), 3.92 (s, 6H), 3.69 (t, J=7.6 Hz, 4H), 3.04 (t, J=7.6 Hz, 4H).

### Step 3: Synthesis of dimethyl 4,5-bis(2-hydroxyethyl)phthalate (Compound 4)

Dimethyl 4,5-bis(2-(benzyloxy)ethyl)phthalate (900 mg) was dissolved in MeOH. Pd/C (150 mg, 10%) was added and the reaction mixture was stirred overnight under H2. The mixture was filtered and concentrated to give crude dimethyl 4,5-bis(2-hydroxyethyl)phthalate (510 mg, 93% yield) as a white solid. ¹H NMR (400 MHz, CDCl₃) 6 7.58 (s, 2H), 3.90 (t, J=6.4 Hz, 4H), 3.89 (s, 6H), 2.99 (t, J=6.6 Hz, 4H), 1.80 (brs, 2H).

### Step 4: Synthesis of dimethyl 4,5-bis(2-((methylsulfonyl)oxy)ethyl)phthalate (Compound 5)

Dimethyl 4,5-bis(2-hydroxyethyl)phthalate (282 mg, 1.0 mmol) and Et₃N (303 mg, 3.0 mmol, 3.0 equiv) was dissolved in DCM (8 mL) and MsCl (286 mg, 2.5 mmol, 2.5 equiv) was added at 0° C. in one portion, then stirred at rt for 45 mins. TLC showed the reaction was complete. DCM was added and the reaction mixture was washed with water, aq. NaHCO₃, brine, dried (Na₂SO₄), and concentrated to give dimethyl 4,5-bis(2-((methylsulfonyl)oxy)ethyl)phthalate (430 mg) that was used in the next step without further purification. ¹H NMR (400 MHz, CDCl₃) δ 7.61 (s, 2H), 4.30 (t, J=7.2 Hz, 4H), 3.91 (s, 6H), 3.18 (t, J=7.2 Hz, 4H), 2.96 (s, 6H).

### Step 5: Synthesis of dimethyl 3 -benzyl-2,3,4,5-tetrahydro-1H-benzo [d]azepine-7,8-dicarboxylate (Compound 6)

Dimethyl 4,5-bis(2-((methylsulfonyl)oxy)ethyl)phthalate (430 mg) was dissolved in 1,2-dichloroethane (10 mL) and benzylamine (1.3 mL, 12 eqiv) was added. The reaction was stirred at 50° C. for 24 h. TLC showes the reaction was complete. DCM was added and the reaction mixture was washed with water, brine, and dried. The resulting crude product was purified by flash column chromatography on silica gel (petroleum ether/EtOAc=10:1 to 1:1) to give dimethyl 3-benzyl-2,3,4,5-tetrahydro-1H-benzo[d]azepine-7,8-dicarboxylate (196 mg). ¹H NMR (400 MHz, CDCl₃) δ 7.44 (s, 2H), 7.36-7.27 (m, 5H), 3.88 (s, 6H), 3.62 (s, 2H), 2.98-2.95 (m, 4H), 2.63-2.61 (m, 4H); LC−MS: [M+H]⁺=354.21

### Step 6: Synthesis of 3-(tert-butyl) 7,8-dimethyl 1,2,4,5-tetrahydro-3H-benzo[d]azepine-3,7,8-tricarboxylatedimethyl (Compound 7)

Dimethyl 3 -benzyl-2,3,4,5-tetrahydro-1H-benzo[d]azepine-7,8-dicarboxylate (190 mg) was dissolved in MeOH, and (Boc)₂O (1.1 equiv) and Pd/C (80 mg, 10% by wt) were added. The reaction mixture was stirred overnight under H₂, and the mixture was filtered and concentrated to give crude 3-(tert-butyl) 7,8-dimethyl 1,2,4,5-tetrahydro-3H-benzo[d]azepine-3,7,8-tricarboxylatedimethyl. ¹H NMR (400 MHz, CDCl₃) δ 7.47 (s, 2H), 7.36-7.27 (m, 5H), 3.88 (s, 6H), 3.55-3.52 (m, 4H), 2.95-2.92 (m, 4H), 1.47 (s, 9H); LC−MS: [M+H]⁺=364.10

### Step 6: Synthesis of tert-butyl 2-(2,6-dioxopiperidin-3-yl)-1,3-dioxo-2,3,5,6,8,9-hexahydroazepino[4,5-f]isoindole-7(1H)-carboxylate (Cpd. No. 855)

3 -(Tert-butyl) 7,8-dimethyl 1,2,4,5-tetrahydro-3H-benzo[d] azepine-3,7,8-tricarboxylate (73 mg, 0.2 mmol) and 3-aminopiperidine-2,6-dione hydrochloride (66 mg, 0.4 mmol, 2 equiv) were dissolved in pyridine (3 mL), and LiI (268 mg, 2 mmol, 10 equiv) was added. The reaction mixture was stirred at 130° C. for 15 h. LC−MS show the reaction was complete. The solvent was removed and purified by preparative HPLC to give Cpd. No. 854. LC−MS:[M+H]⁺=428.30

### Step 7: Synthesis of 2-(2,6-dioxopiperidin-3-yl)-6,7,8,9-tetrahydroazepino[4,5-f]isoindole-1,3(2H,5H)-dione (Cpd. No. 855)

To a solution of tert-butyl 2-(2,6-dioxopiperidin-3-yl)-1,3-dioxo-2,3,5,6,8,9-hexahydroazepino[4,5-f]isoindole-7(1H)-carboxylate in DCM (2 mL) was added TFA (0.5 mL). The reaction mixture was stirred at rt for 1 h and the solvent was removed to give Cpd. No. 855 as the TFA salt. ¹H NMR (400 MHz, DMSO-d₆) δ 11.11 (s, 1H), 9.01 (brs, 2H), 7.83 (s, 2H), 7.79 (s, 1H), 5.13 (dd, J=12.8, 5.4 Hz, 1H), 3.29-3.23 (m, 8H), 2.93-2.85 (m, 1H), 2.63-2.51 (m, 2H), 2.09-2.03 (m, 1H); LC−MS: [M+H]⁺=328.21.

### Example 10

### Synthesis of 2-(2,6-dioxopiperidin-3-yl)-6,7,8,9-tetrahydroazepino[3,4-f]isoindole-1,3(2H,5H)-dione (Cpd. No. 857)

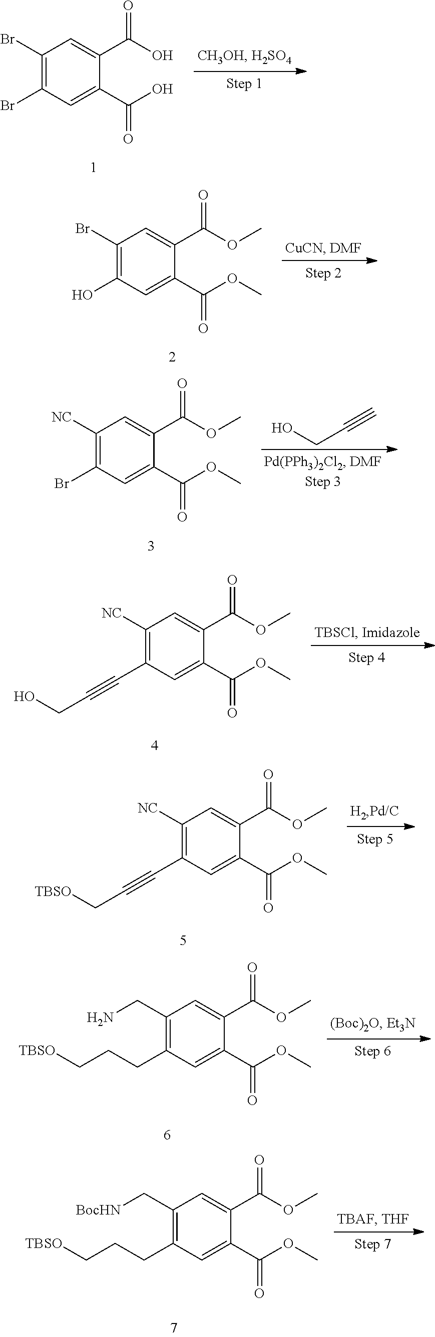

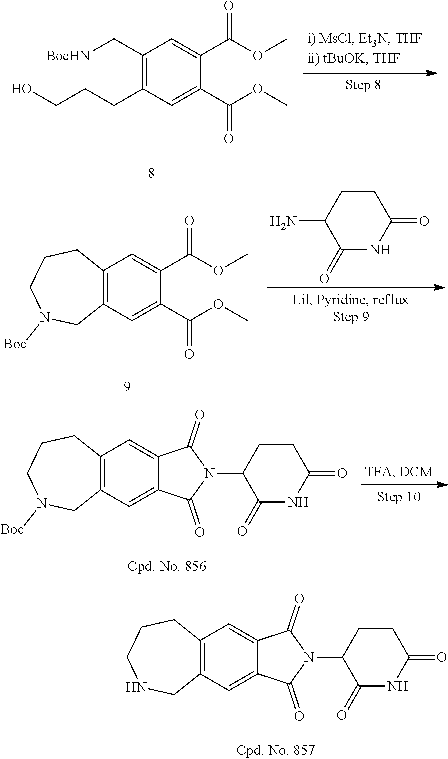 [SMILES: COC(=O)c1cc(CCCO)c(CNC(=O)OC(C)(C)C)cc1C(=O)OC ; COC(=O)c1cc2c(cc1C(=O)OC)CN(C(=O)OC(C)(C)C)CCC2 ; NC1CCC(=O)NC1=O ; CC(C)(C)OC(=O)N1CCCc2cc3c(cc2C1)C(=O)N(C1CCC(=O)NC1=O)C3=O ; O=C1CCC(N2C(=O)c3cc4c(cc3C2=O)CNCCC4)C(=O)N1]

### Step 1: Synthesis of Dimethyl 4,5-dibromophthalate (Compound 2)

To a solution of 4,5-dibromophthalic acid (5 g) and trimethyl orthoformate (25 mL) in MeOH (25 mL) was added conc. H₂SO₄ (2 mL) at room temperature, and the reaction was refluxed overnight. The solvent was removed under vacuum, and EtOAc (100 mL) and sat. aq. NaHCO₃ (20 mL) were added. The reaction mixture was extracted with EtOAc (50 mL×3), and the combined organic extracts were washed with brine, dried (Na₂SO₄), and concentrated in vacuo. The residue was used in the next step without further purification. ¹H NMR (400 MHz, CDCl₃) δ 3.91 (m, 6H), 7.97 (s, 2H).

### Step 2: Synthesis of Dimethyl 4-bromo-5-cyanophthalate (Compound 3)

Dimethyl 4,5-dibromophthalate (1.5 g, 4.28 mmol) and copper(I) cyanide (500 mg, 5.56 mmol) were dissolved with 15 ml of anhydrous DMF and stirred at 100° C. overnight. The reaction mixture was extracted with ethyl ether three times and the organic phase was washed with cold water and brine to remove the excess DMF. Removal of the solvent followed by purification by flash chromatography on silica gel (ethyl acetate-hexane) gave dimethyl 4-bromo-5-cyanophthalate (Compound 3) in 60% yield. LC−MS: [M+H]⁺=297.96.

### Step 3: Synthesis of Dimethyl 4-cyano-5-(3-hydroxyprop-1-yn-1-yl)phthalate (Compound 4)

Compound 3 (1.1 g, 3.71 mmol, 1.0 eq), Pd(PPh₃)₂Cl₂ (263 mg, 0.371 mmol, 0.1 eq), CuI (140 mg, 0.742 nmol. 0.2 eq.), and propargyl alcohol (0.312 g, 5.57 mmol, 1.5 eq.) were dissolved with 15 mL of dry DMF, and the reaction vessel was purged with nitrogen balloon three times. Et₃N (3 mL) was added and the reaction mixture was heated to 80° C. for 2 h. The reaction mixture was extracted with ethyl ether three times and washed with cold water and brine to remove the excess DMF. Removal of the solvent followed by purification by flash chromatography on silica gel (ethyl acetate-hexane) gave dimethyl 4-cyano-5-(3-hydroxyprop-1-yn-1-yl)phthalate (Compound 4) in 70% yield. LC−MS: [M+H]⁺=274.06

### Step 4: Synthesis of Dimethyl 4-(3-((tert-butyldimethylsilyl)oxy)prop-1-yn-1-yl)-5-cyanophthalate (Compound 5)

To a solution of compound 4 (500 mg, 1.83 mmol) and imidazole (373 mg, 5.49 mmol), in dry DCM (10 mL) was added TBSCl (412 mg, 2.74 mmol) under N₂ at room temperature. The reaction mixture was stirred at room temperature for 1 h. The mixture was quenched with H₂O and extracted with DCM. The organic layers were separated and washed with H₂O, brine, dried (MgSO₄), and then purification by flash chromatography on silica gel (ethyl acetate-hexane) to give compound 5 as 90% yield. LC−MS: [M+H]⁺=388.15

### Step 5: Synthesis of Dimethyl 4-(aminomethyl)-5-(3-((tert-butyldimethylsilyl)oxy)propyl)phthalate (Compound 6)

Compound 5 (900 mg) was dissolved in MeOH and Pd/C (90 mg, 10% by wt) was added. The reaction mixture was stirred overnight under H2. The reaction mixture was filtered and concentrated to give crude dimethyl 4-(aminomethyl)-5-(3-((tert-butyldimethylsilyl)oxy)propyl)phthalate (Compound 6). LC−MS: [M+H]⁺=396.21.

### Step 6: Synthesis of Dimethyl 4-(((tert-butoxycarbonyl)amino)methyl)-5-(3-((tert-butyldimethylsilyl)oxy)propyl)phthalate (Compound 7)

Crude compound 6 was dissolved in dry DCM, and Boc₂O (1.1 eq.) and Et₃N (3.0 eq.) were added. The reaction mixture was stirred at room temperature for 2 h. The reaction mixture was concentrated under reduced pressure and purified by flash chromatography on silica gel (ethyl acetate-hexane) to give compound 7 in 60% yield. LC−MS: [M+H]⁺=496.27.

### Step 7: Synthesis of dimethyl 4-(((tert-butoxycarbonyl)amino)methyl)-5-(3-hydroxypropyl)phthalate (Compound 8)

Compound 7 (163 mg, 0.33 mmol) was suspended in dry THF (5 mL) and cooled in an ice bath. TBAF (1 M in THF, 0.66 mL, 0.66 mmol) was added and the reaction mixture was allowed to warm to room temperature and stir for 3 h. The mixture was concentrated in vacuo, diluted with EtOAc, and washed with sat aq. NH₄Cl. The organic layer was concentrated to provide the crude product which was purified by flash chromatography on silica gel (ethyl acetate:hexane=1:1) to give compound 8 in 80% yield. LC−MS: [M+H]⁺=382.18.

### Step 8: Synthesis of 2-(tert-butyl) 7,8-dimethyl 1,3,4,5-tetrahydro-2H-benzo[c]azepine-2,7,8-tricarboxylate (Compound 9)

Compound 8 (200 mg, 0.52 mmol) and Et₃N (131 mg, 1.3 mmol, 2.5 equiv) were dissolved in dry THF (4 mL), and MsCl (89 mg, 0.78 mmol, 1.5 equiv) was added at 0° C. in one portion. The reaction mixture was stirred at rt for 45 min. TLC showed the reaction was complete. The reaction mixture was treated with t-BuOK (1.5 ml 1 (M) THF, 3 equiv) and stirred for an additional 2 h. The reaction mixture was quenched by adding water and extracted with EtOAc. The organic layer was concentrated to provide the crude product which was purified by flash chromatography on silica gel (ethyl acetate:hexane=1:1) to give compound 9 in 60% yield. LC−MS: [M+H]⁺=364.17.

### Step 9: Synthesis of tert-butyl 2-(2,6-dioxopiperidin-3-yl)-1,3-dioxo-2,3,5,7,8,9-hexahydroazepino[3,4-f]isoindole-6(1H)-carboxylate (Cpd. No. 856)

Compound 9 (70 mg, 0.2 mmol) and 3-aminopiperidine-2,6-dione hydrochloride (66 mg, 0.4 mmol, 2 equiv) were dissolved in pyridine (3 mL) and LiI (268 mg, 2 mmol, 10 equiv) was added. The reaction mixture was stirred at 130° C. for 15 h. LC−MS show the reaction was >85% complete. The solvent was removed and the crude product purified by preparative HPLC to give Cpd. No. 856. LC−MS: [M+H]⁺=428.17.

### Step 10: Synthesis 2-(2,6-dioxopiperidin-3-yl)-6,7,8,9-tetrahydroazepino[3,4-f]isoindole-1,3(2H,5H)-dione (Cpd. No. 857)

Cpd. No. 856 (102.1 mg, 0.28 mmol) was added to 1 mL HCl (4M in 1,4-dioxane), and the mixture reaction mixture was stirred at room temperature for 2 h. The 1,4-dioxane was removed under reduced pressure to give Cpd. No. 857 as the HCl salt. LC−MS: [M+H]⁺=328.17. ¹H NMR (400 MHz, MeOH-d₄) δ 7.88 (s, 1H), 7.79 (s, 1H), 5.11 (dd, J=12.6, 5.4 Hz, 1H), 4.54 (s, 2H), 3.54-3.47 (m, 2H), 3.29-3.19 (m, 3H), 2.90-2.62 (m, 3H), 2.16-2.06 (m, 1H), 2.06-1.95 (m, 2H).

### Example 11

### Biological Assays

Compounds of the Disclosure are tested for cereblon inhibition using methods known in the art. For example, Boichenko et al. describe a fluorescence resonance energy transfer (FRET)-based assay for the identification and characterization of cereblon ligands. Boichenko et al., J. Med. Chem. 59:770-774 (2016).

### Example 12

### Synthesis of PROTAC Molecules

Compounds of the Disclosure may be used as monofunctional synthetic intermediates to prepare PROTAC molecules. PROTAC molecules comprising representative Compounds of the Disclosure are disclosed in U.S. Provisional Appl. Nos. 62/902,714, 63/024,697, and 63/024,686.

The synthesis of N-(4-(3-chloro-4-cyanophenoxy)bicyclo[2.2.2]octan-1-yl)-4-(4-(6-(2,6-dioxopiperidin-3-yl)-5,7-dioxo-3,5,6,7-tetrahydropyrrolo[3,4-f]isoindol-2(1H)-yl)piperidin-1-yl)benzamide (Compound B) is shown in Scheme 1.

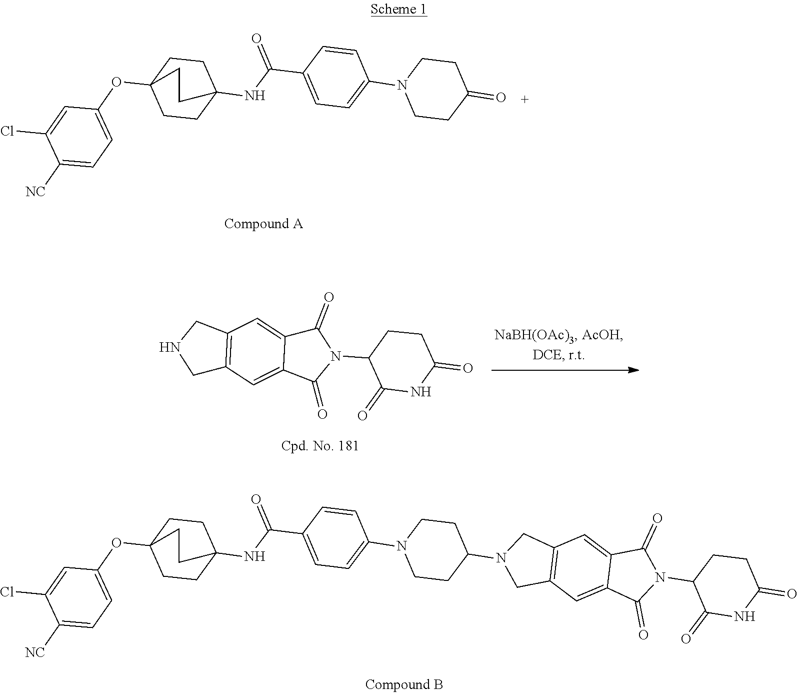 [SMILES: N#Cc1ccc(OC23CCC(NC(=O)c4ccc(N5CCC(=O)CC5)cc4)(CC2)CC3)cc1Cl ; O=C1CCC(N2C(=O)c3cc4c(cc3C2=O)CNC4)C(=O)N1 ; N#Cc1ccc(OC23CCC(NC(=O)c4ccc(N5CCC(N6Cc7cc8c(cc7C6)C(=O)N(C6CCC(=O)NC6=O)C8=O)CC5)cc4)(CC2)CC3)cc1Cl]

To a solution of N-(4-(3-chloro-4-cyanophenoxy)bicyclo[2.2.2]octan-1-yl)-4-(4-oxopiperidin-1-yl)benzamide (Compound A) and 2-(2,6-dioxopiperidin-3-yl)-6,7-dihydropyrrolo[3,4-f]isoindole-1,3(2H,5H)-dione (Cpd. No. 181) in DCE was added NaBH(OAc)₃ and AcOH, the reaction mixture was stirred at r.t. for 6 hours. All volatiles were removed and the residue was chromatographed on silica gel to afford Compound B. ESI-MS: 760.28.

The synthesis of N-((1r,4r)-4-(3-chloro-4-cyanophenoxy)cyclohexyl)-4-(4-(6-(2,6-dioxopiperidin-3-yl)-5,7-dioxo-3,5,6,7-tetrahydropyrrolo[3,4-f]isoindol-2(1H)-yl)piperidin-1-yl)benzamide (Compound D) is shown in Scheme 2.

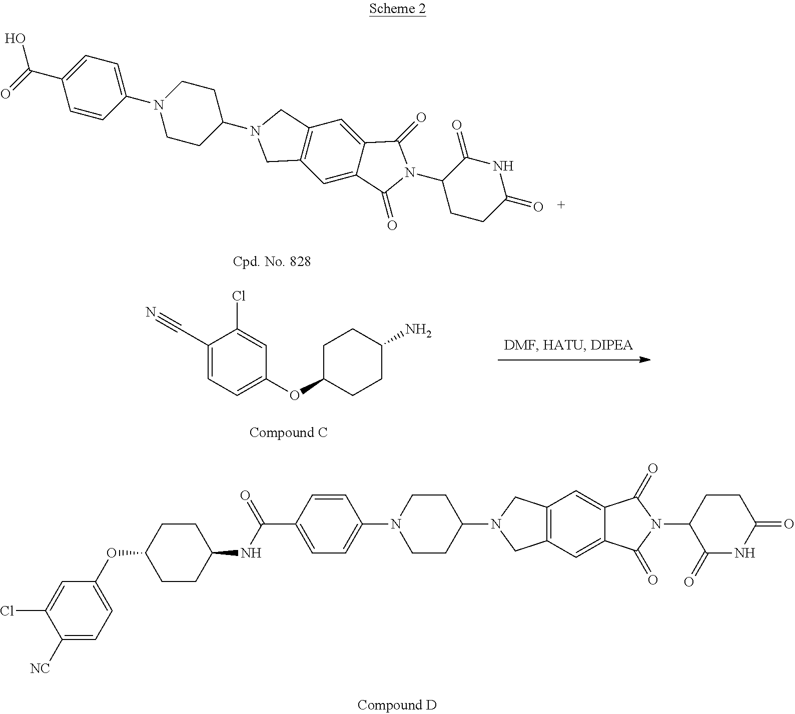 [SMILES: O=C1CCC(N2C(=O)c3cc4c(cc3C2=O)CN(C2CCN(c3ccc(C(=O)O)cc3)CC2)C4)C(=O)N1 ; N#Cc1ccc(O[C@H]2CC[C@H](N)CC2)cc1Cl ; N#Cc1ccc(O[C@H]2CC[C@H](NC(=O)c3ccc(N4CCC(N5Cc6cc7c(cc6C5)C(=O)N(C5CCC(=O)NC5=O)C7=O)CC4)cc3)CC2)cc1Cl]

Cpd. No. 828, see EXAMPLE 3, was dissolved in DMF, and DIPEA (3 eq) and HATU (1.3 eq) were added. Compound C was dissolved in DMF, and DIPEA (3 eq) was added. The Compound C solution was poured into the Cpd. No. 828 solution. The reaction was complete in 0.5 h. The DIPEA was removed, and H2O and TFA (15×) were added. The product was purified by prepative HPLC to give Compound D in 39% yield. UPLC-MS 4.0 min, 735.3.

The synthesis of N-((1r,4r)-4(3-chloro-4-cyanophenyl)(methyl)amino) cyclohexyl)-4-(4-((6-(2,6-dioxopiperidin-3-yl)-5,7-dioxo-3,5,6,7-tetrahydropyrrolo [3,4-f]isoindol-2(1H)-yl)methyl)piperidin-1-yl)benzamide (Compound F) is shown in Scheme 3.

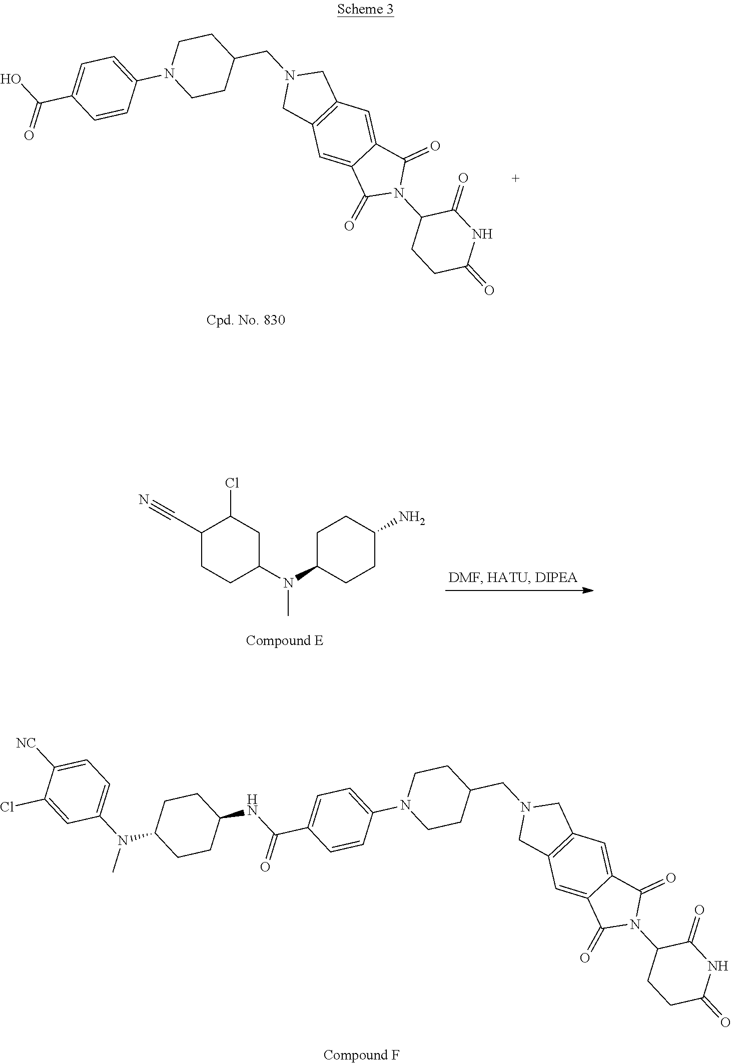 [SMILES: O=C1CCC(N2C(=O)c3cc4c(cc3C2=O)CN(CC2CCN(c3ccc(C(=O)O)cc3)CC2)C4)C(=O)N1 ; CN(C1CCC(C#N)C(Cl)C1)[C@H]1CC[C@H](N)CC1 ; CN(c1ccc(C#N)c(Cl)c1)[C@H]1CC[C@H](NC(=O)c2ccc(N3CCC(CN4Cc5cc6c(cc5C4)C(=O)N(C4CCC(=O)NC4=O)C6=O)CC3)cc2)CC1]

Cpd. No. 830, see EXAMPLE 4, was dissolved in DMF, and DIPEA (3 eq) and HATU (1.3 eq) were added. Compound E was dissolved in DMF, and DIPEA (3 eq) was added. The Compound E solution was poured into the Cpd. No. 830 solution. The reaction was complete in 0.5 h. The DIPEA was removed, and H₂O and TFA (15×) were added. The product was purified by prepative HPLC to give Compound F in 41% yield. UPLC-MS 4.1 min, 762.35.

### Example 13

### Synthesis of 2-(2,6-dioxopiperidin-3-yl)-7,8-dihydropyrrolo[3,4-e]isoindole-1,3(2H,6H)-dione (Cpd. No. 481)

The synthesis of Cpd. No. 481 is shown in Scheme 4.

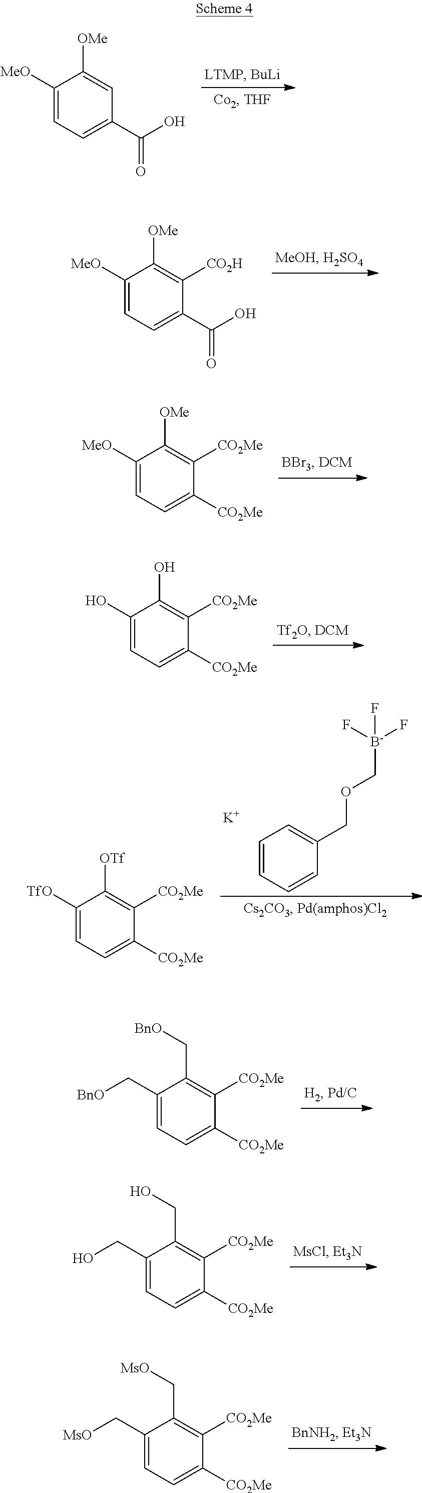

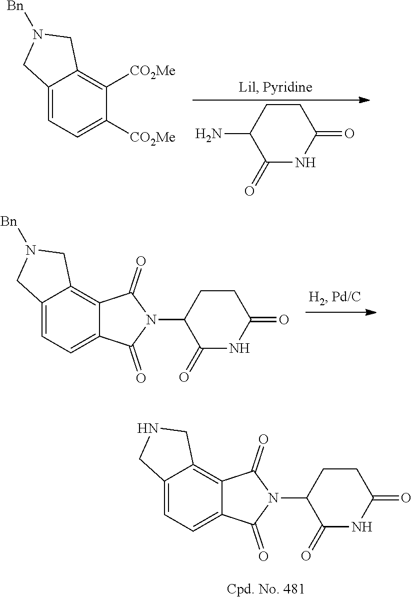 [SMILES: COC(=O)c1ccc2c(c1C(=O)OC)CN(Cc1ccccc1)C2 ; NC1CCC(=O)NC1=O ; O=C1CCC(N2C(=O)c3ccc4c(c3C2=O)CN(Cc2ccccc2)C4)C(=O)N1 ; O=C1CCC(N2C(=O)c3ccc4c(c3C2=O)CNC4)C(=O)N1]

### Example 14

### Synthesis of 2-(2,6-dioxopiperidin-3-yl)-7,8,9,10-tetrahydroazepino[4,5-e]isoindole-1,3(2H,6H)-dione (Cpd. No. 859)

The synthesis of Cpd. No. 859 is shown in Scheme 5.

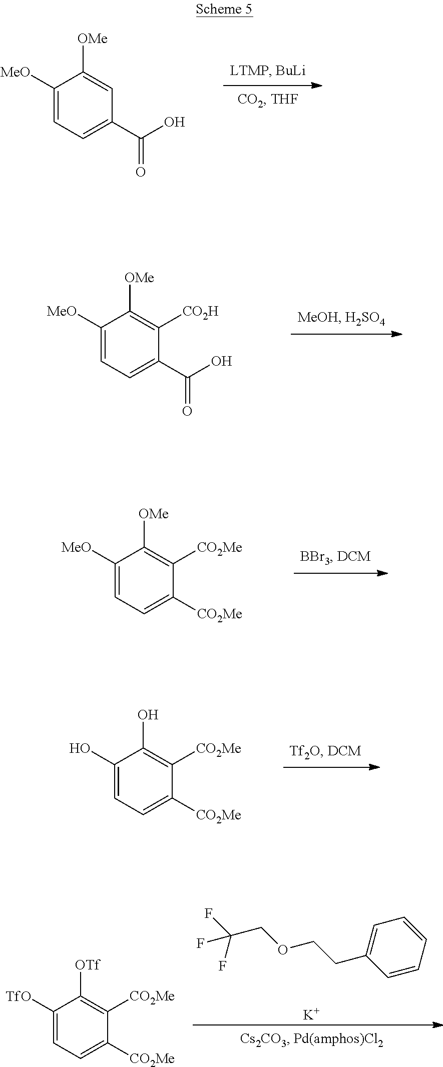

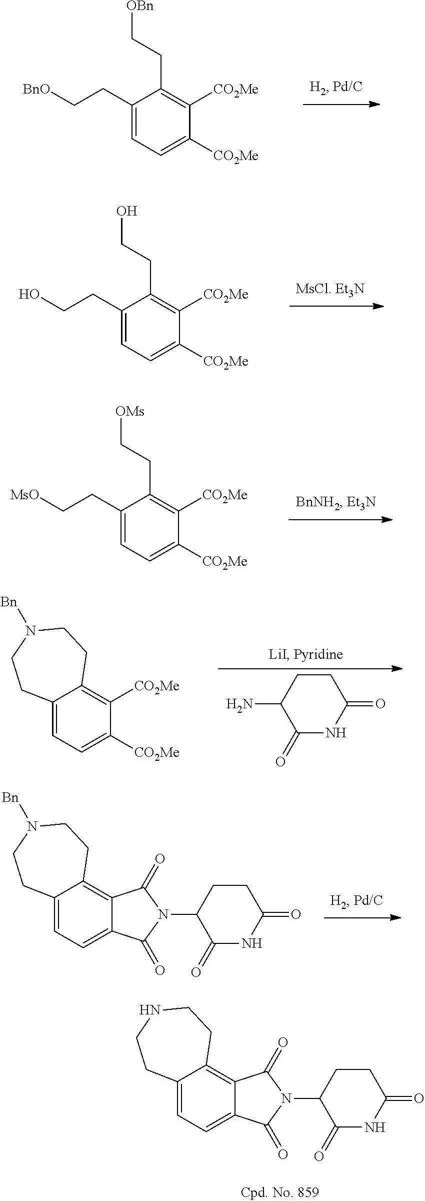

### Example 15

### Synthesis of 2-(2,6-dioxopiperidin-3-yl)-6,7,8,9-tetrahydro-1H-pyrrolo[3,4-h]isoquinoline-1,3(2H)-dione (Cpd. No. 601)

The synthesis of Cpd. No. 601 is shown in Scheme 6.

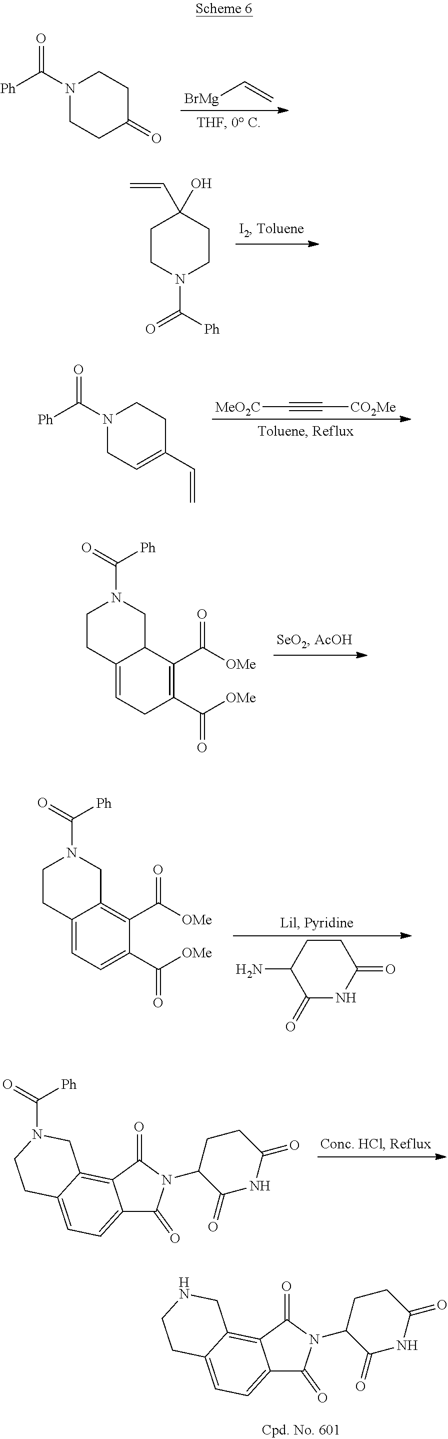

### Example 16

### Synthesis of 2′-(2,6-dioxopiperidin-3-yl)-5′,7′-dihydro-1′H-spiro[azetidine-3,6′-cyclopenta[f]isoindole]-1′,3′(2′H)-dione (Cpd. No. 827)

The synthesis of Cpd. No. 827 is shown in Scheme 7.

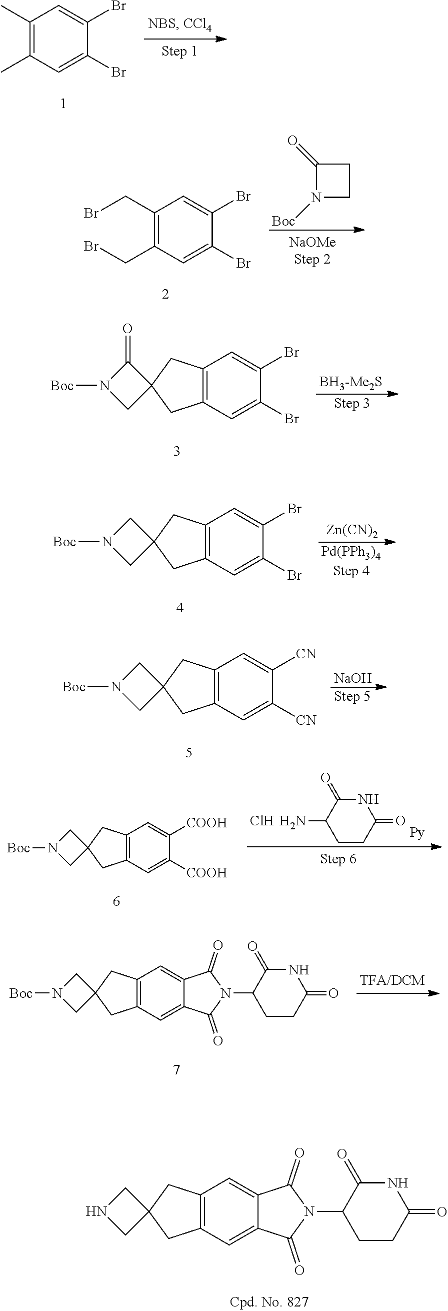

### Example 17

### Synthesis of 2-(2,6-dioxopiperidin-3-yl)-5,7-dihydro-1H-spiro[cyclopenta[f]isoindole-6,4′-piperidine]-1,3(2H)-dione (Cpd. No. 827a) and 2′-(2,6-dioxopiperidin-3-yl)-5′,7′-dihydro-1′H-spiro[azepane-4,6′-cyclopenta[f]isoindole]-1′,3′(2′H)-dione (Cpd. No. 861)

The synthesis of Cpd. Nos. 827a and 861 are shown in Scheme 8.

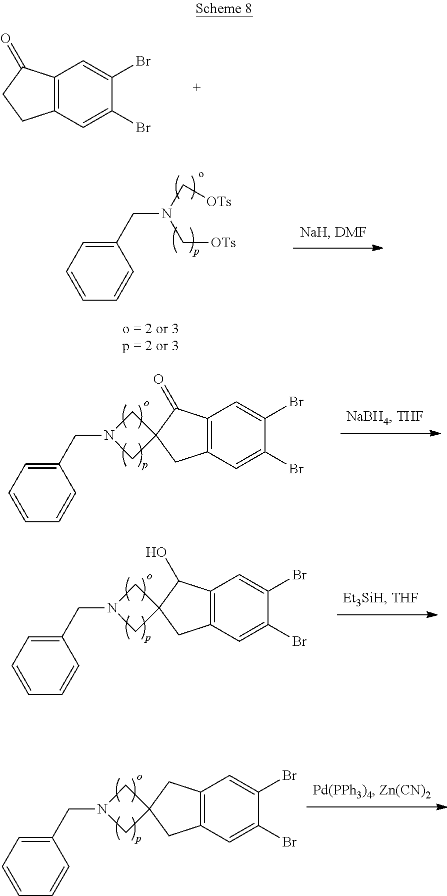

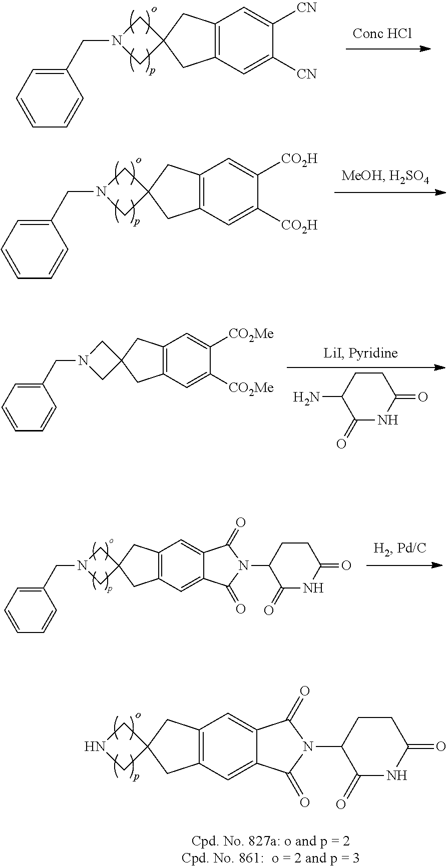

### Example 18

### Synthesis of 2-(2,6-dioxopiperidin-3-yl)-5,7-dihydro-1H-spiro[cyclopenta[f]isoindole-6,3′-pyrrolidine]-1,3(2H)-dione (Cpd. No. 860)

The synthesis of Cpd. No. 860 is shown in Scheme 9.

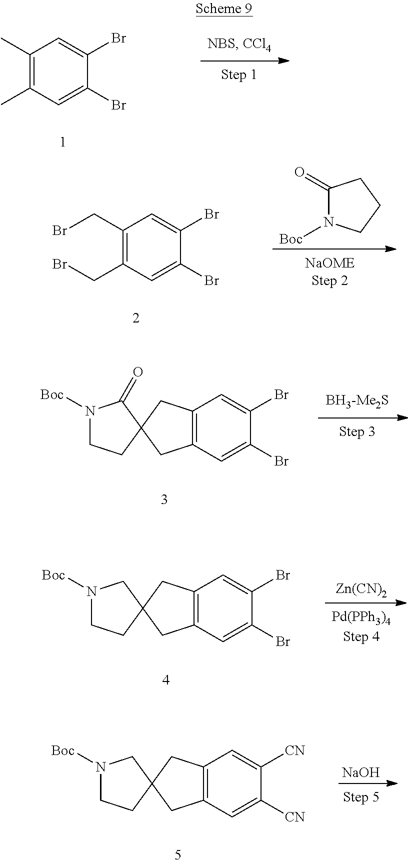

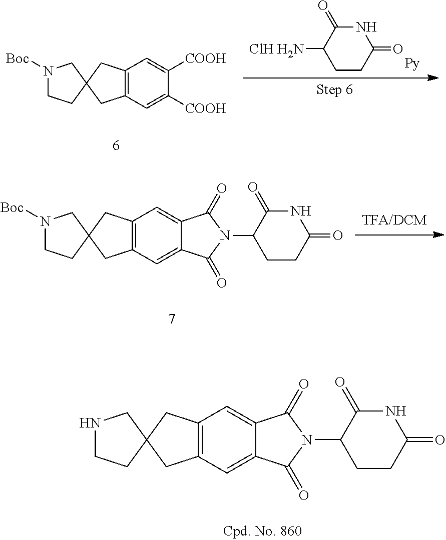

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof.

All patents, patent applications, and publications cited herein are fully incorporated by reference herein in their entirety.

What is claimed is:

1. A compound of Formula I: 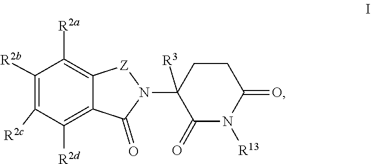
wherein:
R^{2b} and R^{2c} are taken together to form a —(CH₂)ₘ—N(R¹)—(CH₂)ₙ- radical, a —(CH₂)ₘ—C(R^{1a})(R^{1b})—(CH₂)ₙ—radical, or a 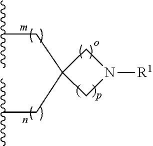
radical; and R^{2a} and R^{2d} are independently selected from the group consisting of hydrogen, halo, C₁-C₃ alkyl, and C₁-C₃ alkoxy; or
R^{2a} and R^{2b} are taken together to form a —(CH₂)ₘ—N(R¹)—(CH₂)ₙ— radical, a —(CH₂)ₘ—C(R^{1a})(R^{1b})—(CH₂)ₙ radical, or a 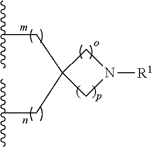
radical; and R^{2c} and R^{2d} are independently selected from the group consisting of hydrogen, halo, C₁-C₃ alkyl, and C₁-C₃ alkoxy; or
R^{2c} and R^{2d} are taken together to form a —(CH₂)ₘ—N(R¹)—(CH₂)ₙ— radical,
a —(CH₂)ₘ—C(R^{1a})(R^{1b})—(CH₂)ₙ—radical, or a 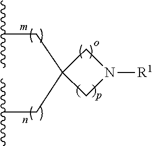
radical; and R^{2a} and R^{2b} are independently selected from the group consisting of hydrogen, halo, C₁-C₃ alkyl, and C₁-C₃ alkoxy;
R³ is selected from the group consisting of hydrogen, deuterium, fluoro, and C₁-C₃ alkyl;
m is 1, 2, or 3;
n is 1, 2, or 3;
o is 1, 2, or 3;
p is 1, 2, or 3;
Z is selected from the group consisting of —CR^{8a}R^{8b}— and —C(═O)—;
R¹ is selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷;
R^{1a} is selected from the group consisting of hydrogen, —OH, —CHO, —C(═O)OH, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷;
R^{1b} is selected from the group consisting of hydrogen and C₁-C₃ alkyl; or R^{1a} and R^{1b} taken together with the carbon atom to which they are attached form a —C(═O)—;
R⁴ is selected from the group consisting of —R^{4a}, —OR^{4b}, and —NR^{4c}R^{4d.};
R⁵ is selected from the group consisting of —R^{5a} and —NR^{5b}R^{5c.};
R⁶ is selected from the group consisting of hydrogen, C₁-C₆ alkyl, and cyano;
R⁷ is selected from the group consisting of hydrogen, C₁-C₆ alkyl, and —NR^{7a}R^{7b};
R^{4a} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{4b} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{4c} and R^{4d} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl; or
R^{4c} and R^{4d} taken together with the nitrogen atom to which they are attached form a 4- to 8-membered optionally substituted heterocyclo;
R^{5a} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{5b} and R^{5c} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{7a} and R^{7b} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl; or
R^{7a} and R^{7b} taken together with the nitrogen atom to which they are attached form a 4- to 8-membered optionally substituted heterocyclo;
R^{8a} and R^{8b} are independently selected from the group consisting of hydrogen and C₁-C₃ alkyl; or
R^{8a} and R^{8b} taken together with the carbon atom to which they are attached from a C₃-C₆ cycloalkyl; and
R¹³ is selected from the group consisting of hydrogen and C₁-C₃ alkyl, or a pharmaceutically acceptable salt or solvate thereof.

2. The compound of claim 1 of Formula II: 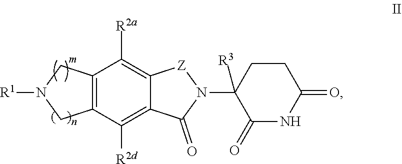
or a pharmaceutically acceptable salt or solvate thereof.

3. The compound of claim 2, wherein Z is —CH₂—, or a pharmaceutically acceptable salt or solvate thereof.

4. The compound of claim 2, wherein Z is —C(═O)—, or a pharmaceutically acceptable salt or solvate thereof.

5. The compound of any one of claims 2-4, wherein R^{2a} and R^{2d} are independently selected from the group consisting of hydrogen, fluoro, and chloro, or a pharmaceutically acceptable salt or solvate thereof.

6. The compound of claim 1 of Formula III: 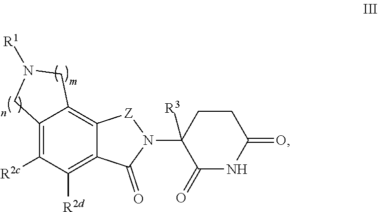
or a pharmaceutically acceptable salt or solvate thereof.

7. The compound of claim 6, wherein Z is —CH₂—, or a pharmaceutically acceptable salt or solvate thereof.

8. The compound of claim 6, wherein Z is —C(═O)—, or a pharmaceutically acceptable salt or solvate thereof.

9. The compound of any one of claims 6-8, wherein R^{2c} and R^{2d} are independently selected from the group consisting of hydrogen, fluoro, and chloro, or a pharmaceutically acceptable salt or solvate thereof.

10. The compound of claim 1 of Formula IV: 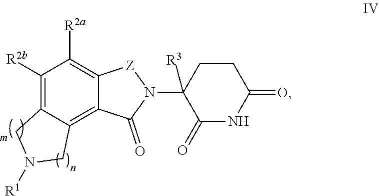
wherein Z is —CR^{8a}R^{8b}—, or a pharmaceutically acceptable salt or solvate thereof.

11. The compound of claim 10, wherein Z is —CH₂—, or a pharmaceutically acceptable salt or solvate thereof.

12. The compound of claim 10 or 11, wherein R^{2a} and R^{2b} are independently selected from the group consisting of hydrogen, fluoro, and chloro, or a pharmaceutically acceptable salt or solvate thereof.

13. The compound of claim 1 of Formula IX: 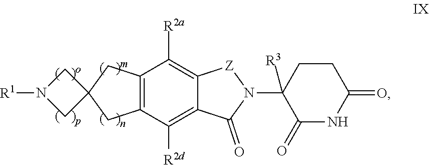
or a pharmaceutically acceptable salt or solvate thereof.

14. The compound of claim 13, wherein Z is —CH₂—, or a pharmaceutically acceptable salt or solvate thereof.

15. The compound of claim 13, wherein Z is —C(═O)—, or a pharmaceutically acceptable salt or solvate thereof.

16. The compound of any one of claims 13-15, wherein R^{2a} and R^{2d} are independently selected from the group consisting of hydrogen, fluoro, and chloro, or a pharmaceutically acceptable salt or solvate thereof.

17. The compound of any one of claims 13-16, wherein o is 1 or 2; and p is 1 or 2, or a pharmaceutically acceptable salt or solvate thereof.

18. The compound of claim 1 of Formula X: 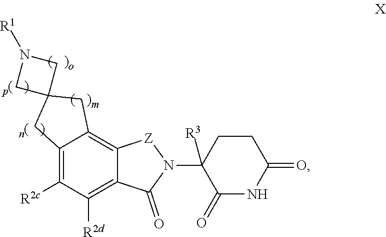
or a pharmaceutically acceptable salt or solvate thereof.

19. The compound of claim 18, wherein Z is —CH₂—, or a pharmaceutically acceptable salt or solvate thereof.

20. The compound of claim 18, wherein Z is —C(═O)—, or a pharmaceutically acceptable salt or solvate thereof.

21. The compound of any one of claims 18-20, wherein R^{2c} and R^{2d} are independently selected from the group consisting of hydrogen, fluoro, and chloro, or a pharmaceutically acceptable salt or solvate thereof.

22. The compound of any one of claims 18-21, wherein o is 1 or 2; and p is 1 or 2, or a pharmaceutically acceptable salt or solvate thereof.

23. The compound of claim 1 of Formula XI: 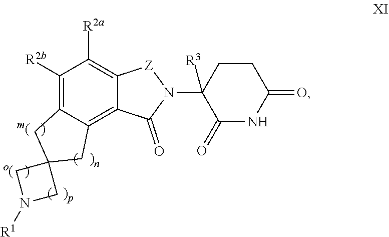
wherein Z is —^{8a} R^{8b}—, or a pharmaceutically acceptable salt or solvate thereof.

24. The compound of claim 23, wherein Z is —CH₂—, or a pharmaceutically acceptable salt or solvate thereof.

25. The compound of claim 23 or 24, wherein R^{2c} and R^{2d} are independently selected from the group consisting of hydrogen, fluoro, and chloro, or a pharmaceutically acceptable salt or solvate thereof.

26. The compound of any one of claims 23-25, wherein o is 1 or 2; and p is 1 or 2, or a pharmaceutically acceptable salt or solvate thereof.

27. The compound of claim 1 of Formula XIV: 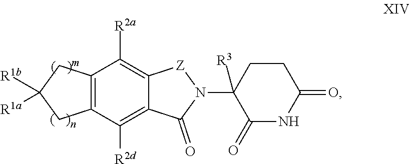
or a pharmaceutically acceptable salt or solvate thereof.

28. The compound of claim 27, wherein Z is —CH₂—, or a pharmaceutically acceptable salt or solvate thereof.

29. The compound of claim 27, wherein Z is —C(═O)—, or a pharmaceutically acceptable salt or solvate thereof.

30. The compound of any one of claims 27-29, wherein R^{2a} and R^{2d} are independently selected from the group consisting of hydrogen, fluoro, and chloro, or a pharmaceutically acceptable salt or solvate thereof

31. The compound of any one of claims 27-30, wherein R^{1a} is selected from the group consisting of —OH, —CHO, —CH₂OH, and —C(═O)OH; and R^{1b} is hydrogen, or a pharmaceutically acceptable salt or solvate thereof.

32. The compound of any one of claims 27-30, wherein R^{1a} and R^{1b} taken together with the carbon atom to which they are attached form a —C(═O)—, or a pharmaceutically acceptable salt or solvate thereof.

33. The compound of any one of claims 1-32, wherein R³ is selected from the group consisting of hydrogen, deuterium, fluoro, and methyl, or a pharmaceutically acceptable salt or solvate thereof.

34. The compound of any one of claims 1-33, wherein m is 1, or a pharmaceutically acceptable salt or solvate thereof.

35. The compound of any one of claims 1-33, wherein m is 2, or a pharmaceutically acceptable salt or solvate thereof.

36. The compound of any one of claims 1-35, wherein n is 1, or a pharmaceutically acceptable salt or solvate thereof.

37. The compound of any one of claims 1-35, wherein n is 2, or a pharmaceutically acceptable salt or solvate thereof.

38. A compound of Formula XVIII: 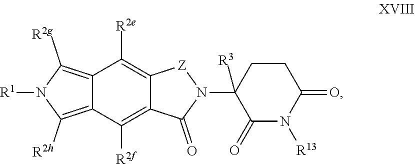
wherein:
R^{2e}, R^{2f}, R^{2g}, and R^{2h} are independently selected from the group consisting of hydrogen, halo, C₁-C₃ alkyl, and C₁-C₃ alkoxy;
Z is selected from the group consisting of —CR^{8a}R^{8b}— and C(═O)—;
R¹ is selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy) alkyl, (amino)alkyl, (alkoxy)alkyl, (cyclo alkyl) alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷;
R³ is selected from the group consisting of hydrogen, deuterium, fluoro, and C₁-C₃ alkyl;
R⁴ is selected from the group consisting of —R^{4a}, —OR^{4b}, and—NR^{4c}R^{4d};
R⁵ is selected from the group consisting of —R^{5a} and —NR^{5b}R^{5c};
R⁶ is selected from the group consisting of hydrogen, C₁-C₆ alkyl, and cyano;
R⁷ is selected from the group consisting of hydrogen, C₁-C₆ alkyl, and —NR^{7a}R^{7b};
R^{4a} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy) alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{4b} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{4c} and R^{4d} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl; or
R^{4c} and R^{4d} taken together with the nitrogen atom to which they are attached form a 4- to 8-membered optionally substituted heterocyclo;
R^{5a} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{5b} and R^{5c} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{7a} and R^{7b} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl; or
R^{7a} and R^{7b} taken together with the nitrogen atom to which they are attached form a 4- to 8-membered optionally substituted heterocyclo;
R^{8a} and R^{8b} are independently selected from the group consisting of hydrogen and C₁-C₃ alkyl; or
R^{8a} and R^{8b} taken together with the carbon atom to which they are attached from a C₃-C₆ cycloalkyl; and
R¹³ is selected from the group consisting of hydrogen and C₁-C₃ alkyl,
or a pharmaceutically acceptable salt or solvate thereof.

39. The compound of claim 38, wherein Z is —CH₂—, or a pharmaceutically acceptable salt or solvate thereof.

40. The compound of claim 38, wherein Z is —C(═O)—, or a pharmaceutically acceptable salt or solvate thereof.

41. The compound of any one of claims 38-40, wherein R³ is hydrogen, or a pharmaceutically acceptable salt or solvate thereof.

42. The compound of any one of claims 38-41, wherein R¹³ is hydrogen, or a pharmaceutically acceptable salt or solvate thereof.

43. The compound of any one of claims 38-43, wherein R^{2e}, R^{2f}, R^{2g}, and R^{2h} are hydrogen, or a pharmaceutically acceptable salt or solvate thereof.

44. The compound of any one of claim 1-26 or 33-43, wherein R¹ is hydrogen, or a pharmaceutically acceptable salt or solvate thereof.

45. The compound of any one of claim 1-26 or 33-43, wherein R¹ is selected from the group consisting of optionally substituted C₁-C₆ alkyl, C₁-C₆ haloalkyl, optionally substituted C₃-C₈ cycloalkyl, or a pharmaceutically acceptable salt or solvate thereof.

46. The compound of claim 45, wherein R¹ is optionally substituted C₁-C₆ alkyl, or a pharmaceutically acceptable salt or solvate thereof.

47. The compound of any one of claim 1-26 or 33-43, wherein R¹ is selected from the group consisting of (hydroxy)alkyl, (amino)alkyl, (alkoxy)alkyl, (cycloalkyl)alkyl, (heterocyclo)alkyl, (heteroaryl)alkyl, and aralkyl, or a pharmaceutically acceptable salt or solvate thereof.

48. The compound of claim 47, wherein R¹ is (heterocyclo)alkyl, or a pharmaceutically acceptable salt or solvate thereof.

49. The compound of any one of claim 1-26 or 33-43, wherein R¹ is selected from the group consisting of optionally substituted 4- to 8-membered heterocyclo, optionally substituted aryl, and optionally substituted heteroaryl, or a pharmaceutically acceptable salt or solvate thereof.

50. The compound of claim 49, wherein R¹ is optionally substituted 4- to 6-membered heterocyclo.

51. The compound of any one of claim 1-26 or 33-43, wherein R¹ is —C(═O)R⁴, or a pharmaceutically acceptable salt or solvate thereof.

52. The compound of claim 41, wherein R⁴ is —OR^{4b}; and R^{4b} is C₁-C₆ alkyl, or a pharmaceutically acceptable salt or solvate thereof.

53. The compound of any one of claim 1-26 or 33-43, wherein R¹ is —S(═O)₂R⁵, or a pharmaceutically acceptable salt or solvate thereof.

54. The compound of any one of claim 1-26 or 33-43, wherein R¹ is —C(═NR⁶)R⁷, or a pharmaceutically acceptable salt or solvate thereof.

55. The compound of claim 2 that is any one or more of the compounds of Table 1, or a pharmaceutically acceptable salt or solvate thereof.

56. The compound of claim 6 that is any one or more of the compounds of Table 2, or a pharmaceutically acceptable salt or solvate thereof.

57. The compound of claim 10 that is any one or more of the compounds of Table 3, or a pharmaceutically acceptable salt or solvate thereof.

58. The compound of claim 27 that is any one or more of the compounds of Table 8, or a pharmaceutically acceptable salt or solvate thereof.

59. The compound of claim 1 of Formula XII: 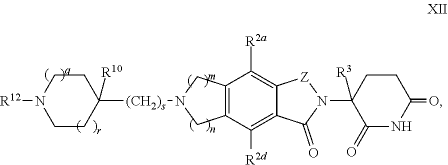
wherein:
q and r are independently 0, 1, or 2;
s is 0 or 1;
R¹⁰ is selected from the group consisting of hydrogen, halo, C₁-C₃ alkyl, and C₁-C₃ alkoxy; and
R¹² is selected from the group consisting of hydrogen, optionally substituted heterocylo, and optionally substituted phenyl, or a pharmaceutically acceptable salt or solvate thereof.

60. The compound of claim 53 of Formula XIII: 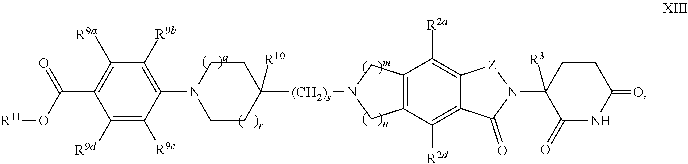
wherein:
R^{9a}, R^{9b}, R^{9c}, and R^{9d} are independently selected from the group consisting of hydrogen, halo, C₁-C₃ alkyl, C₁-C₃ haloalkyl, and C₁-C₃ alkoxy; and
R¹¹ is selected from the group consisting of hydrogen and C₁-C₆ alkyl,
or a pharmaceutically acceptable salt or solvate thereof.

61. The compound of claim 38 of Formula XXI: 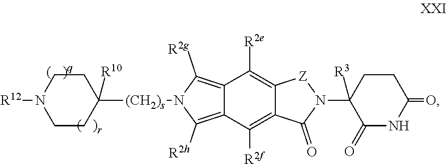
wherein:
q and r are independently 0, 1, or 2;
s is 0 or 1;
R¹⁰ is selected from the group consisting of hydrogen, halo, C₁-C₃ alkyl, and C₁-C₃ alkoxy; and
R¹² is selected from the group consisting of hydrogen, optionally substituted heterocylo, and optionally substituted phenyl, or a pharmaceutically acceptable salt or solvate thereof.

62. The compound of claim 61 of Formula XXII: 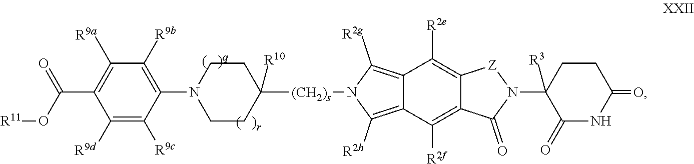
wherein:
R^{9a}, R^{9b}, R^{9c},and R^{9d} are independently selected from the group consisting of hydrogen, halo, C₁-C₃ alkyl, C₁-C₃ haloalkyl, and C₁-C₃ alkoxy; and
R¹¹ is selected from the group consisting of hydrogen and C₁-C₆ alkyl,
or a pharmaceutically acceptable salt or solvate thereof.

63. A pharmaceutical composition comprising the compound of any one of claims 1-62, or a pharmaceutically acceptable salt or solvate thereof, and a pharmaceutically acceptable carrier.

64. A method of treating cancer in a subject in need thereof, the method comprising administering to the subject a therapeutically effective amount of the compound of any one of claims 1-62, or a pharmaceutically acceptable salt or solvate thereof.

65. The method of claim 64, wherein the cancer is any one or more of the cancers of Table 5.

66. The method of claim 64 or 65 further comprising administering a therapeutically effective amount of an optional therapeutic agent useful in the treatment of cancer.

67. The pharmaceutical composition of claim 63 for use in treating cancer.

68. The pharmaceutical composition of claim 67, wherein the cancer is any one or more of the cancers of Table 5.

69. A compound of any one of claims 1-62, or a pharmaceutically acceptable salt or solvate thereof, for use in treating of cancer.

70. The compound for use of claim 69, wherein the cancer is any one or more of the cancers of Table 5.

71. Use of a compound of any one of claims 1-62, or a pharmaceutically acceptable salt or solvate thereof, for the manufacture of a medicament for treatment of cancer.

72. The use of claim 71, wherein the cancer is any one or more of the cancers of Table 5.

73. A method of inhibiting CRBN ubiquitination within a cell of a subject in need thereof, the method comprising administering to the subject a compound of any one of claims 1-62, or a pharmaceutically acceptable salt or solvate thereof.

74. A kit comprising the compound of any one of claims 1-62, or a pharmaceutically acceptable salt or solvate thereof, and instructions for administering the compound, or a pharmaceutically acceptable salt or solvate thereof, to a subject having cancer.

75. The kit of claim 74, wherein the cancer is any one or more of the cancers of Table 4.

76. A compound of Formula VI: 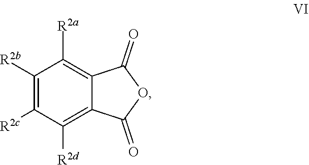
wherein:
R^{2b} and R^{2c} are taken together to form a —(CH₂)ₘ—N(R¹)—(CH₂)ₙ— radical, a —(CH₂)ₘ—C(R^{1a})(R^{1b})—(CH₂)ₙ— radical, or a 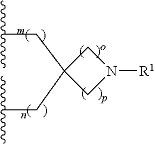
radical; and R^{2a} and R^{2d} are independently selected from the group consisting of hydrogen, halo, C₁-C₃ alkyl, and C₁-C₃ alkoxy; or
R^{2a} and R^{2b} are taken together to form a —(CH₂)ₘ—N(R¹)—(CH₂)ₙ— radical, a —(CH₂)ₘ—C(R^{1a})(R^{1b})—(CH₂)ₙ— radical, or a 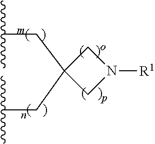
radical; and R^{2c} and R^{2d} are independently selected from the group consisting of hydrogen, halo, C₁-C₃ alkyl, and C₁-C₃ alkoxy; or
R^{2c} and R^{2d} are taken together to form a —(CH₂)ₘ—N(R¹)—(CH₂)ₙ— radical, a —(CH₂)ₘ—C(R^{1a} )(R^{1b})—(CH₂)ₙ— radical, or a 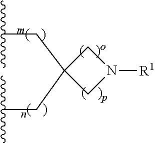
radical; and R^{2a} and R^{2b} are independently selected from the group consisting of hydrogen, halo, C₁-C₃ alkyl, and C₁-C₃ alkoxy;
m is 1, 2, or 3;
n is 1, 2, or 3;
o is 1, 2, or 3;
p is 1, 2, or 3
R¹ is selected from the group consisting optionally substituted C₁-C₆ alkyl, optionally substituted C₂-C₆ alkenyl, optionally substituted C₂-C₆ alkynyl, C₁-C₆ haloalkyl, (hydroxy) alkyl, (amino)alkyl, (alkoxy)alkyl, (cyclo alkyl) alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷;
R^{1a} is selected from the group consisting of hydrogen, —OH, —CHO, —C(═O)OH, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷;
R^{1b} is selected from the group consisting of hydrogen and C₁-C₃ alkyl; or R^{1a} and R^{1b} taken together with the carbon atom to which they are attached form a —C(═O)—;
R⁴ is selected from the group consisting of —R^{4a}, —OR^{4b}, and —NR^{4c}R^{4d.};
R⁵ is selected from the group consisting of —R^{5a} and —NR^{5b}R^{5c};
R⁶ is selected from the group consisting of hydrogen, C₁-C₆ alkyl, and cyano;
R⁷ is selected from the group consisting of hydrogen, C₁-C₆ alkyl, and —NR^{7a}R^{7b.};
R^{4a} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{4b} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{4c} and R^{4d} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl; or
R^{4c} and R^{4d} taken together with the nitrogen atom to which they are attached form a 4- to 8-membered optionally substituted heterocyclo;
R^{5a} is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl;
R^{5b} and R^{5c} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl; and
R^{7a} and R^{7b} are independently selected from the group consisting of hydrogen, optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, aralkyl, optionally substituted C₃-C₈ cycloalkyl, optionally substituted 4- to 10-membered heterocyclo, optionally substituted aryl, and optionally substituted heteroaryl; or
R^{7a} and R^{7b} taken together with the nitrogen atom to which they are attached form a 4- to 8-membered optionally substituted heterocyclo,
or a salt or solvate thereof.

77. A method of making a compound of claim 1, wherein the method comprising:
(i) reacting a compound of Formula V: 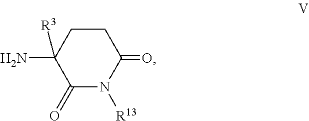
or a salt thereof;
with compound of Formula VI: 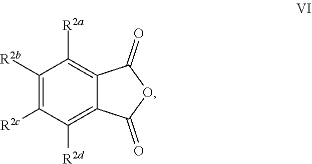
in a solvent, wherein Z is —C(═O)—; and R¹ is selected from the group consisting of optionally substituted C₁-C₆ alkyl, optionally substituted C₂-C₆ alkenyl, optionally substituted C₂-C₆ alkynyl, C₁-C₆ haloalkyl, (hydroxy)alkyl, (amino)alkyl, (alkoxy)alkyl, (cycloalkyl)alkyl, (heterocyclo)alkyl, (heteroaryl)alkyl, aralkyl, optionally substituted C₃-C₈ cycloalkyl, optionally substituted 4- to 10-membered heterocyclo, optionally substituted aryl, optionally substituted heteroaryl, —C(═O)R⁴, —S(═O)₂R⁵, and —C(═NR⁶)R⁷.
